(19) United States
(12) Patent Application Publication
(10) Pub. No.: US 2025/0171837 A1
(43) Pub. Date: May 29, 2025

(54) COMPOSITON FOR LIQUEFYING VISCOUS BIOLOGICAL SAMPLE, COMBINTATION PRODUCT, LIQUEFYING AGENT, AND KIT THEREOF, AND METHOD AND APPLICATION THEREOF

(71) Applicant: SANSURE BIOTECH INC., Changsha (CN)

(72) Inventors: Lizhong DAI, Changsha (CN); Zhingping DENG, Changsha (CN); Shiyao CHEN, Changsha (CN); Yong DENG, Changsha (CN); Jia LIU, Changsha (CN)

(21) Appl. No.: 18/681,762

(22) Filed: Feb. 6, 2024

Related U.S. Application Data
(63) Continuation of application No. PCT/CN2022/110828, filed on Aug. 8, 2022.

(30) Foreign Application Priority Data
Aug. 6, 2021 (CN) 202110901455.0
Nov. 3, 2021 (CN) 202111294356.7
Jan. 27, 2022 (CN) 202210098137.X
Feb. 16, 2022 (CN) 202210143116.5

(51) Int. Cl. C12Q 1/6851; C12Q 1/6806

(52) U.S. Cl. CPC C12Q 1/6851; C12Q 1/6806

(57) ABSTRACT

Disclosed is a viscous biological sample liquefying composition comprising guaifenesin and a first strong base, further relates to a combination product, a liquefying agent and a kit containing the viscous biological sample liquefying composition, and further relates to a liquefaction method, a processing method and application of the viscous biological sample using the viscous biological sample liquefying composition or the combination product or kit thereof.

### CROSS-REFERENCE TO RELATED APPLICATIONS

This application is a continuation of International Application No. PCT/CN2022/110828, filed on Aug. 8, 2022, which claims priority to Chinese Patent Application No. CN2021109014550, filed on Aug. 6, 2021, Chinese Patent Application No. CN2021112943567, filed on Nov. 3, 2021, Chinese Patent Application No. CN202210098137X, filed on Jan. 27, 2022, and Chinese Patent Application No. CN2022101431165, filed on Feb. 16, 2022. All of the aforementioned patent applications are hereby incorporated by reference in their entireties.

### TECHNICAL FIELD

This disclosure relates to the technical field of biological sample processing and nucleic acid detection, in particular to viscous biological sample liquefying composition, combination product, liquefying agent, and kit thereof, as well as methods and application thereof.

### BACKGROUND

With the rapid development of technologies such as fluorescent quantitative PCR and multiplex PCR, etc., in the field of pathogen detection, there is an increasing demand to detect whether the corresponding pathogen species are infected by the types of sputum samples. However, sputum samples have the characteristics of high viscosity, more proteins and complex components, including mucin and other proteins (such as immune proteins), a variety of enzymes, exfoliated cells, microorganisms and other inhaled impurities, etc., which are not convenient for direct detection. Clinically, the detection of sputum samples requires liquefaction treatment of sputum first.

The common methods of sputum liquefaction are sodium hydroxide method, DTT (dithiothreitol) method and protease method. Sodium hydroxide method is the most commonly used sputum liquefaction method. This method is relatively simple, sodium hydroxide solution with a certain concentration as the main component is used to liquefy sputum at 60° C.-80° C. (or at room temperature). This method, due to its strong alkaline environment, leads to easy loss of nucleic acids when used for nucleic acid detection. The principle of protease method is to use protease to digest mucin in sputum. This method is time-consuming in enzymatic hydrolysis reaction and of high cost for protease, and has low digestion efficiency of mucin. DTT (dithiothreitol) method, as the most commonly used sputum liquefaction method at present, comprises breaking mucin, the main component in sputum causing viscosity, by using DTT (dithiothreitol) containing sulfhydryl (—SH), providing physiological buffer using PBS buffer (phosphate buffer), and usually adding ethanol and the like to fix cells. This method is time-consuming, and DTT (dithiothreitol) used is expensive, and has poor stability, and is needed to be preserved at low temperature. Meanwhile, DTT has certain toxicity, which is not suitable for mass treatment of sputum samples clinically.

Therefore, there is an urgent need to develop new liquefying agents and methods suitable for viscous biological samples such as sputum, etc.

### SUMMARY

In view of the above technical problems, this disclosure provides a viscous biological sample liquefying composition, which can quickly liquefy the viscous biological sample, reduce the viscosity of the sample, facilitate the subsequent operations, and perform at room temperature with less rigorous operating conditions and without affecting the subsequent nucleic acid detection. It is compatible with the extraction free amplification system, and has no obvious impact on the preservation time of nucleic acids in the sample. This disclosure also provides a combination product, a liquefying agent and a kit containing the viscous biological sample liquefying composition, and further provides a liquefaction method, a processing method and application of the viscous biological sample using the viscous biological sample liquefying composition or the combination product or kit thereof.

In a first aspect of this disclosure, there is provided a viscous biological sample liquefying composition, comprising guaifenesin and a first strong base.

In some embodiments, the first strong base is at least one of sodium hydroxide, potassium hydroxide, lithium hydroxide, calcium hydroxide, barium hydroxide or choline; further preferably, the first strong base is sodium hydroxide, potassium hydroxide or a combination thereof.

In some embodiments, the viscous biological sample liquefying composition further comprises rigid microparticles;

preferably, the rigid microparticles are made of a material including at least one of zirconia, silicon nitride, ceramsite, hard stainless steel, hard tungsten carbide, sintered corundum or agate;

further preferably, the rigid microparticles are of a shape including one or more of sphere, near-sphere, ellipsoid, column, rod, polyhedron and irregular shape; further preferably, the rigid microparticles are of a shape being sphere, near-sphere, ellipsoid, column, rod, polyhedron or irregular shape; further preferably, the rigid microparticles are of a shape including one or more of sphere, cube, polyhedron and irregular shape; further preferably, the rigid microparticles are of a shape being sphere, cube, polyhedron or irregular shape;

further preferably, the rigid microparticles are of an average particle size of 0.01 mm to 500 mm; further preferably 0.05 mm to 500 mm; further preferably 0.1 mm to 500 mm; further preferably 0.2 mm to 500 mm; further preferably 0.5 mm to 500 mm; further preferably 1 mm to 500 mm; further preferably 0.01 mm to 200 mm; further preferably 0.05 mm to 200 mm; further preferably 0.1 mm to 200 mm; further preferably 0.2 mm to 200 mm; further preferably 0.5 mm to 200 mm; further preferably 1 mm to 200 mm; further preferably 0.01 mm to 100 mm; further preferably 0.05 mm to 100 mm; further preferably 0.1 mm to 100 mm; further preferably 0.2 mm to 100 mm; further preferably 0.5 mm to 100 mm; further preferably 1 mm to 100 mm; further preferably 0.01 mm to 50 mm; further preferably 0.05 mm to 50 mm; further preferably 0.1 mm to 50 mm; further preferably 0.2 mm to 50 mm; further preferably 0.5 mm to 50 mm; further preferably 1 mm to 50 mm; further preferably 0.01 mm to 10 mm; further preferably 0.05 mm to 10 mm; further preferably 0.1 mm to 10 mm; further preferably 0.2 mm to 10 mm; further preferably 0.5 mm to 10 mm; further preferably 1 mm to 10 mm; further preferably 0.01 mm to 5 mm; further preferably 0.05 mm to 5 mm; further preferably 0.1 mm to 5 mm; further preferably 0.2 mm to 5 mm; further preferably 0.5 mm to 5 mm; further preferably 1 mm to 5 mm;

further preferably, the rigid microparticles are applied in an amount of 0.1 g/mL to 2 g/mL; further preferably 0.2 g/mL to 2 g/mL; further preferably 0.5 g/mL to 2 g/mL; further preferably 0.8 g/mL to 2 g/mL; further preferably 1 g/mL to 2 g/mL; further preferably 0.1 g/mL to 1.5 g/mL; further preferably 0.2 g/mL to 1.5 g/mL; further preferably 0.5 g/mL to 1.5 g/mL; further preferably 0.8 g/mL to 1.5 g/mL; further preferably 1 g/mL to 1.5 g/mL; further preferably 0.1 g/mL to 1.2 g/mL; further preferably 0.2 g/mL to 1.2 g/mL; further preferably 0.5 g/mL to 1.2 g/mL; further preferably 0.8 g/mL to 1.2 g/mL; further preferably 1 g/mL to 1.2 g/mL; further preferably 0.1 g/mL to 1 g/mL; further preferably 0.2 g/mL to 1.5 g/mL; further preferably 0.5 g/mL to 1 g/mL; further preferably 0.8 g/mL to 1 g/mL.

In a second aspect of this disclosure, there is provided a liquefying agent comprising the viscous biological sample liquefying composition of the first aspect of this disclosure;

wherein the guaifenesin is of a concentration of 1 mmol/L to 1 mol/L and the first strong base is of a concentration less than 1 mol/L, preferably 0.1 mmol/L to 500 mmol/L;

preferably, the liquefying agent further comprises a first aqueous solvent.

In some embodiments, the guaifenesin in the liquefying agent is of a concentration of 20 mmol/L to 1 mol/L; preferably 20 mmol/L to 500 mmol/L; further preferably 20 mmol/L to 400 mmol/L; further preferably 20 mmol/L to 200 mmol/L; further preferably 20 mmol/L to 150 mmol/L; further preferably 50 mmol/L to 400 mmol/L; further preferably 50 mmol/L to 200 mmol/L; further preferably 50 mmol/L to 150 mmol/L; further preferably 50 mmol/L, 100 mmol/L, 150 mmol/L or 200 mmol/L.

In some embodiments, the first strong base in the liquefying agent is of a concentration of 1 mmol/L to 500 mmol/L; preferably 5 mmol/L to 500 mmol/L; further preferably 5 mmol/L to 400 mmol/L; further preferably 5 mmol/L to 200 mmol/L; further preferably 5 mmol/L to 100 mmol/L; further preferably 5 mmol/L to 80 mmol/L; further preferably 5 mmol/L to 60 mmol/L; further preferably 5 mmol/L to 50 mmol/L; further preferably 5 mmol/L to 40 mmol/L; further preferably 10 mmol/L to 200 mmol/L; further preferably 10 mmol/L to 100 mmol/L; further preferably 10 mmol/L to 50 mmol/L; further preferably 10 mmol/L to 40 mmol/L; further preferably 40 mmol/L, 50 mmol/L, 60 mmol/L, 80 mmol/L, or 100 mmol/L.

In some embodiments, the liquefying agent is of a pH value equal to or greater than 10, preferably 10 to 14, further preferably 10 to 12 or 12 to 14, further preferably 10, 10.5, 11, 11.5, 12, 12.5, 13, 13.5 or 14.

In a third aspect of this disclosure, there is provided a viscous biological sample processing combination product, comprising a liquefaction component, and further comprising a preservation component, a nucleic acid releasing component or a combination thereof; wherein the liquefaction component comprises the viscous biological sample liquefying composition of the first aspect of this disclosure; or the liquefaction component is in an agent combination or a single liquefying agent; preferably, the viscous biological sample processing combination product comprises the liquefaction component and the preservation component;
further preferably, the viscous biological sample processing combination product comprises the liquefaction component and the nucleic acid releasing component;
further preferably, the viscous biological sample processing combination product comprises the liquefaction component, the preservation component and the nucleic acid releasing component;
further preferably, the viscous biological sample processing combination product comprises the single liquefying agent;
further preferably, the viscous biological sample processing combination product comprises a preservation agent;
further preferably, the viscous biological sample processing combination product comprises a releasing agent;
further preferably, the viscous biological sample processing combination product comprises the single liquefying agent and the preservation agent;
further preferably, the viscous biological sample processing combination product comprises the single liquefying agent and the releasing agent;
further preferably, the viscous biological sample processing combination product comprises the single liquefying agent, the preservation agent and the releasing agent;
wherein the single liquefying agent is selected from the liquefying agent of the second aspect of this disclosure; the preservation agent is a mixture consisted of the preservation component; and the releasing agent is a mixture consisted of the nucleic acid releasing component.

In some embodiments, the preservation component comprises:
a1) a buffer component, for adjusting a preservation system to be a pH value of 6 to 8;
b1) an osmotic pressure regulating component; and
c1) at least one of trehalose, mannitol or glycerol;
preferably, the preservation component constitutes the preservation agent.

In some embodiments, the preservation component satisfies one or more of the following characteristics:
(tb1) the buffer is for adjusting the preservation system to be a pH value of 6.8 to 7.6; further preferably 6.8 to 7.4; further preferably 6.8 to 7.2; further preferably 7.0 to 7.6; further preferably 7.0 to 7.4; further preferably 7.0 to 7.2; further preferably 7.2 to 7.4;
(tb2) the buffer component comprises citric acid;
(tb3) the osmotic pressure regulating component comprises betaine, inorganic cation or a combination thereof; preferably, the inorganic cation comprises Na⁺, K⁺ or a combination thereof; further preferably, the inorganic cation is Na⁺, K⁺ or a combination thereof;
further preferably, the osmotic pressure regulating component comprises sodium chloride and potassium chloride; further preferably, the osmotic pressure regulating component is a combination of sodium chloride and potassium chloride;
(tb4) the preservation component comprises trehalose, mannitol and glycerol;
(tb5) the preservation component further comprises one or more amino acids; and
(tb6) the preservation component further comprises urea.

In some embodiments, the preservation component satisfies one or more of the following characteristics:
(tb2a) the buffer component comprises 1 mmol/L to 5 mmol/L of citric acid; preferably, the buffer comprises 1.5 mmol/L to 4.5 mmol/L of citric acid; further preferably, the buffer comprises 2 mmol/L to 4 mmol/L of citric acid; further preferably, the buffer comprises 2.5 mmol/L to 3.5 mmol/L of citric acid; further preferably, the buffer comprises 1 mmol/L to 5 mmol/L of citric acid;
(tb3a) the osmotic pressure regulating component comprises 0.1% to 1.2% (m/v) of sodium chloride and 0.1% to 1.2% (m/v) of potassium chloride;
(tb5a) the preservation component further comprises one or more amino acids and the amino acids in the preservation component are of a total concentration of 1 mol/L to 3 mol/L; further preferably, the amino acids in the preservation component are of a total concentration of 1.5 mol/L to 3 mol/L or 1 mol/L to 2.5 mol/L; further preferably, the amino acids in the preservation component are of a total concentration of 1.5 mol/L to 2.5 mol/L;
further preferably, the preservation component comprises glycine and isoleucine; further preferably, the preservation component comprises 0.8 mol/L to 1 mol/L of glycine and 0.6 mol/L to 1 mol/L of isoleucine;
further preferably, the amino acids in the preservation component are glycine and isoleucine; further preferably, the amino acids in the preservation component are 0.8 mol/L to 1 mol/L of glycine and 0.6 mol/L to 1 mol/L of isoleucine;
(tb4a) the trehalose in the preservation component is of a concentration of 0.5 mol/L to 1 mol/L;
(tb4b) the mannitol in the preservation component is of a concentration of 1.5% to 4.5% (m/v);
(tb4c) the glycerol in the preservation component is of a concentration of 2% to 10% (v/v); and
(tb6a) the preservation component further comprises 1% to 3% (m/v) of urea;
preferably, the preservation component comprises 1 mmol/L to 5 mmol/L of citric acid, 0.1% to 1.2% (w/v) of sodium chloride, 0.1% to 1.2% (w/v) of potassium chloride, 0.8 mol/L to 1 mol/L of glycine, 0.6 mol/L to 1 mol/L of isoleucine, 0.5 mol/L to 1 mol/L of trehalose, 1.5% to 4.5% (w/v) of mannitol and 2% to 10% (v/v) of glycerol. In some embodiments, the nucleic acid releasing component is a composition of component i), a composition of component ii), or a composition of component i) and component ii); component i) comprises: 0.1% to 2% (v/v) of Tween 20, 0.1% to 3% (v/v) of Triton X-100, 0.1% to 3% (v/v) of ethyl phenyl polyethylene glycol, 50 mmol/L to 1.25 mol/L of a second strong base, a first adsorbing agent and a second aqueous solvent; component ii) comprises: 0.01 mmol/L to 0.5 mmol/L of surfactant, 0.01% to 2% (w/v) of dodecylbenzene sulfonate, 0.05% to 1% (v/v) of ethanol and 100 mmol/L to 1.25 mol/L of a third strong base, with or without a second adsorbing agent;
further preferably, the liquefaction component constitutes the liquefying agent.

In some embodiments, the nucleic acid releasing component satisfies one or more of the following characteristics: (tr1) the first adsorbing agent comprises a chelating resin, trehalose or a combination thereof; further preferably, the first adsorbing agent comprises Chelex resin, trehalose or a combination thereof; further preferably, the first adsorbing agent is a chelating resin; further preferably, the first adsorbing agent is Chelex resin;
further preferably, the Chelex resin in the component i) is of a concentration of 1% to 15% (w/v);
(tr2) the first adsorbing agent comprises trehalose; preferably, the trehalose in the component i) is of a concentration of 0.5 mol/L to 1 mol/L, further preferably 0.7 mol/L to 1 mol/L;
(tr3) the second adsorbing agent is Chelex resin; further preferably, the Chelex resin in the component ii) is of a concentration of 1% to 15% (w/v);
(tr4) the second strong base in the component i) is at least one of sodium hydroxide, potassium hydroxide, lithium hydroxide, calcium hydroxide, barium hydroxide or choline; preferably, the second strong base is sodium hydroxide, potassium hydroxide or a combination thereof;
(tr5) the third strong base in the component ii) is at least one of sodium hydroxide, potassium hydroxide, lithium hydroxide, calcium hydroxide, barium hydroxide or choline; preferably, the third strong base is sodium hydroxide, potassium hydroxide or a combination thereof;
(tr6) the second aqueous solvent in the component i) is 0.5 mmol/L to 500 mmol/L of Tris-HCl; and
(tr7) the surfactant in the component ii) is one or more of Surfactin, sodium lauryl sulfate and ethylenediamine tetraacetic acid.

In some embodiments, the component i) further comprises 20 mmol/L to 1 mol/L of a second inorganic cation; preferably, the second inorganic cation is Na⁺, K⁺ or a combination thereof;
the component ii) further comprises 50 mmol/L to 1.2 mol/L of a third inorganic cation; preferably, the third inorganic cation is Na⁺, K⁺ or a combination thereof; preferably, the component ii) comprises 0.01 mmol/L to 0.5 mmol/L of the surfactant, 0.01% to 1% (w/v) dodecylbenzene sulfonate, 60 mmol/L to 1 mol/L of the third inorganic cation, 0.05% to 1% (v/v) of ethanol, 150 mmol/L to 1.25 mol/L of the third strong base, and the second adsorbing agent.

In a fourth aspect of this disclosure, there is provided a viscous biological sample liquefying agent comprising a liquefaction component, a Surfactin and a fourth aqueous solvent;
wherein the liquefaction component comprises the viscous biological sample liquefying composition of the first aspect of this disclosure;
the viscous biological sample liquefying agent is of a pH value equal to or greater than 10.

In some embodiments, the viscous biological sample liquefying agent comprises 20 mmol/L to 1 mol/L of guaifenesin, the first strong base, 20 mmol/L to 200 mmol/L of Surfactin and the fourth aqueous solvent;
the viscous biological sample liquefying agent is compatible with an amplification system, preferably a DNA direct amplification type, an RNA direct amplification type or a DNA/RNA direct amplification type.

In some embodiments, the viscous biological sample liquefying agent satisfies one or more of the following characteristics:
(tz1) the guaifenesin and the Surfactin are of a mass ratio of (1 to 5):1, preferably (2 to 5):1, further preferably (1 to 3):1, further preferably (1.5 to 2.5):1, further preferably 1:1, 1.5:1, 2:1, 2.5:1, 3:1, 3.5:1, 4:1, 4.5:1 or 5:1;
(tz2) the guaifenesin is of a concentration of 20 mmol/L to 500 mmol/L; preferably 80 mmol/L to 120 mmol/L; further preferably 20 mmol/L to 400 mmol/L; further preferably 20 mmol/L to 200 mmol/L; further preferably 20 mmol/L to 150 mmol/L; further preferably 50 mmol/L to 400 mmol/L; further preferably 50 mmol/L to 200 mmol/L; further preferably 50 mmol/L to 150 mmol/L;
(tz3) the Surfactin is of a concentration of 20 mmol/L to 150 mmol/L; preferably, the Surfactin is of a concentration of 30 mmol/L to 60 mmol/L;
(tz4) the first strong base is sodium hydroxide, potassium hydroxide or a combination thereof;
(tz5) the viscous biological sample liquefying agent is of a pH value equal to or greater than 10, preferably 10 to 14, further preferably 10 to 12 or 12 to 14, further preferably 10, 10.5, 11, 11.5, 12, 12.5, 13, 13.5 or 14;
(tz6) the fourth aqueous solvent in the viscous biological sample liquefying agent is water or a fourth buffer component;
preferably, the fourth aqueous solvent is RNase-free water; further preferably, the fourth aqueous solvent is nuclease-free water;
preferably, the fourth buffer is one or a combination selected from Tris-HCl, potassium dihydrogen phosphate-sodium hydroxide buffer, disodium hydrogen phosphate-potassium dihydrogen phosphate buffer, and disodium hydrogen phosphate-citric acid buffer; and
(tz7) the viscous biological sample liquefying agent optionally comprises one or both of Na⁺ and K⁺;
preferably, Na⁺ and K⁺ in the viscous biological sample liquefying agent each independently have a concentration of 1 mmol/L to 1 mol/L, preferably 5 mmol/L to 1 mol/L, further preferably 5 mmol/L to 500 mmol/L, further preferably 5 mmol/L to 200 mmol/L;
further preferably, Na⁺ and K⁺ are of a total concentration of 1 mmol/L to 250 mmol/L, preferably 1 mmol/L to 200 mmol/L, further preferably 5 mmol/L to 250 mmol/L, further preferably 5 mmol/L to 200 mmol/L, further preferably 5 mmol/L to 150 mmol/L, further preferably 5 mmol/L to 120 mmol/L;
further preferably, the viscous biological sample liquefying agent comprises 80 mmol/L to 120 mmol/L of guaifenesin, 8 mmol/L to 12 mmol/L of sodium hydroxide, 0.5 g/mL to 1.5 g/mL of zirconia beads with an average particle size of 0.1 mm to 10 mm, 5% to 10% (w/v) of an adsorbing agent (denoted as a fourth adsorbing agent), 30 mmol/L to 60 mmol/L of Surfactin, and the fourth aqueous solvent.

In some embodiments, the viscous biological sample liquefying agent further comprises one or both of an adsorbing agent and rigid microparticles;

preferably, the adsorbing agent is a chelating resin;
further preferably, the adsorbing agent is Chelex resin;
further preferably, the adsorbing agent is in an amount of 1% to 15% (w/v) in the viscous biological sample liquefying agent; further preferably, the adsorbing agent is Chelex resin;
further preferably, the rigid microparticles are made of a material including at least one of zirconia, silicon nitride, ceramsite, hard stainless steel, hard tungsten carbide, sintered corundum or agate;
further preferably, the rigid microparticles are of a shape being sphere, near-sphere, ellipsoid, column, rod, polyhedron or irregular shape; preferably, the rigid microparticles are of a shape being sphere, cube, polyhedron or irregular shape;
further preferably, the rigid microparticles are of an average particle size of 0.01 mm to 10 mm; further preferably 0.05 mm to 10 mm; further preferably 0.1 mm to 10 mm; further preferably 0.2 mm to 10 mm; further preferably 0.5 mm to 10 mm; further preferably 1 mm to 10 mm; further preferably 0.01 mm to 5 mm; further preferably 0.05 mm to 5 mm; further preferably 0.1 mm to 5 mm; further preferably 0.2 mm to 5 mm; further preferably 0.5 mm to 5 mm; further preferably 1 mm to 5 mm;
further preferably, the rigid microparticles are applied in an amount of 0.1 g/mL to 2 g/mL; further preferably 0.2 g/mL to 2 g/mL; further preferably 0.5 g/mL to 2 g/mL; further preferably 0.8 g/mL to 2 g/mL; further preferably 1 g/mL to 2 g/mL; further preferably 0.1 g/mL to 1.5 g/mL; further preferably 0.2 g/mL to 1.5 g/mL; further preferably 0.5 g/mL to 1.5 g/mL; further preferably 0.8 g/mL to 1.5 g/mL; further preferably 1 g/mL to 1.5 g/mL; further preferably 0.1 g/mL to 1.2 g/mL; further preferably 0.2 g/mL to 1.2 g/mL; further preferably 0.5 g/mL to 1.2 g/mL; further preferably 0.8 g/mL to 1.2 g/mL; further preferably 1 g/mL to 1.2 g/mL; further preferably 0.1 g/mL to 1 g/mL; further preferably 0.2 g/mL to 1.5 g/mL; further preferably 0.5 g/mL to 1 g/mL; further preferably 0.8 g/mL to 1 g/mL.

In a fifth aspect of this disclosure, there is provided a combination product for liquefying and preserving viscous biological sample comprising a liquefaction component and a preservation component;
wherein the liquefaction component comprises each component in the viscous biological sample liquefying agent of the fourth aspect of this disclosure;
preferably, the preservation component is as defined in the viscous biological sample processing combination product of the third aspect of this disclosure.

In a sixth aspect of this disclosure, there is provided a kit comprising at least one of the viscous biological sample liquefying composition of the first aspect of this disclosure, the liquefying agent of the second aspect of this disclosure, the viscous biological sample processing combination product of the third aspect of this disclosure, the viscous biological sample liquefying agent of the fourth aspect of this disclosure, and the combination product for liquefying and preserving viscous biological sample of the fifth aspect of this disclosure;
preferably, the kit further comprises a nucleic acid processing component,
further preferably, the nucleic acid processing component comprises one, two or three of a nucleic acid extraction agent, a nucleic acid amplification agent, and a nucleic acid detection agent.

In some embodiments, the kit satisfies one or more of the following characteristics:
(tk1) the kit comprises the viscous biological sample liquefying composition, and further comprises any one or more of a preservation agent, a releasing agent, an extraction agent, an amplification agent and a detection agent which are suitable for nucleic acids;
preferably, the nucleic acids comprise one or both of DNA and RNA;
(tk2) the kit comprises a combination product for liquefying and preserving viscous biological sample, wherein the combination product for liquefying and preserving viscous biological sample comprises the liquefaction component and the preservation agent in the viscous biological sample processing combination product of the third aspect of this disclosure, and
the kit further comprises any one or more of a releasing agent, an extraction agent, an amplification agent and a detection agent which are suitable for nucleic acids; preferably, the nucleic acids comprise one or both of DNA and RNA;
(tk3) the kit comprises a combination product for liquefying and releasing viscous biological sample, wherein the combination product for liquefying and releasing viscous biological sample comprises the liquefaction component and the nucleic acid releasing component in the viscous biological sample processing combination product of the third aspect of this disclosure, the kit further comprises one or more of a preservation agent, an extraction agent, an amplification agent and a detection agent which are suitable for nucleic acids; preferably, the nucleic acids comprise one or both of DNA and RNA;
(tk4) the kit comprises the viscous biological sample liquefying agent of the fourth aspect of this disclosure, and the kit further comprises one or more of a preservation agent, an amplification agent and a detection agent which are suitable for nucleic acids; preferably, the nucleic acids comprise one or both of DNA and RNA; and (tk5) the kit comprises the combination product for liquefying and preserving viscous biological sample of the fifth aspect of this disclosure, and the kit further comprises one or more of an amplification agent and a detection agent which are suitable for nucleic acids; preferably, the nucleic acids comprise one or both of DNA and RNA.

In a seventh aspect of this disclosure, there is provided a method for liquefying a viscous biological sample, comprising steps of: mixing the viscous biological sample with a liquefaction component to obtain a first mixture, and incubating the first mixture; wherein:
when the liquefaction component is the viscous biological sample liquefying composition of the first aspect of this disclosure or the liquefying agent of the second aspect of this disclosure, a second mixture is obtained after incubation;
when the liquefaction component is selected from the viscous biological sample liquefying agent of the fourth aspect of this disclosure, a liquefied product is obtained after incubation;
preferably, the conditions for incubation satisfy one or more of the following characteristics:
(tc1) a pH value is equal to or greater than 10, preferably 10 to 14, further preferably 10 to 12 or 12 to 14, further preferably 10, 10.5, 11, 11.5, 12, 12.5, 13, 13.5 or 14;
(tc2) the guaifenesin in the first mixture is of a concentration of 1 mmol/L to 1 mol/L; preferably 20 mmol/L to 1 mol/L; further preferably 20 mmol/L to 500 mmol/L; further preferably 20 mmol/L to 400 mmol/L; further preferably 20 mmol/L to 200 mmol/L; further preferably 20 mmol/L to 150 mmol/L; further preferably 50 mmol/L to 400 mmol/L; further preferably 50 mmol/L to 200 mmol/L; further preferably 50 mmol/L to 150 mmol/L;
further preferably 50 mmol/L, 100 mmol/L, 150 mmol/L or 200 mmol/L;
(tc3) the mixing is performed by stirring or shaking;
(tc4) a temperature for incubation is selected from 18 to 35° C., preferably 20 to 35° C., further preferably 25 to 35° C., further preferably 18 to 30° C., further preferably 20 to 30° C., further preferably 25 to 30° C., further preferably 18° C., 20° C., 21° C., 22° C., 23° C., 24° C., 25° C., 26° C., 27° C., 28° C., 29° C., 30° C., 31° C., 32° C., 33° C., 34° C. or 35° C.;
(tc5) a time for incubation is less than 30 mins, preferably less than 15 mins, further preferably 2 mins to 15 mins, further preferably 3 mins to 15 mins, further preferably 2 mins to 10 mins, further preferably 3 mins to 10 mins, further preferably 2 mins to 8 mins, further preferably 3 mins to 8 mins, further preferably 2 mins to 6 mins, further preferably 3 mins to 6 mins, further preferably 2 mins to 5 mins, further preferably 3 mins to 5 mins, further preferably 2 mins, 3 mins, 4 mins, 5 mins, 6 mins, 7 mins, 8 mins, 9 mins, 10 mins or 15 mins.

In some embodiments, the viscous biological sample is sputum, cervical mucus, nasopharyngeal swab or oral swab.

In an eighth aspect of this disclosure, there is provided a method for processing viscous biological sample, comprising a step of liquefying the viscous biological sample by using the method for liquefying a viscous biological sample of the seventh aspect of this disclosure;
the method is any one selected from a liquefaction and preservation method, a liquefaction and releasing method, an extraction method, an amplification method and a detection method;
the liquefaction and preservation method comprises a step of: mixing the second mixture or the liquefied product with the preservation component in the viscous biological sample processing combination product of the third aspect of this disclosure to obtain a liquefied sample-preserving solution;
preferably, a volume ratio of the preservation component to the second mixture or the liquefied product is 1:(2 to 4), further preferably 1:2, 1:2.5, 1:3. 1:3.5 or 1:4;
when the second mixture is used, a liquefied sample-preserving solution I is obtained;
when the liquefied product is used, a liquefied sample-preserving solution II is obtained;
the liquefaction and releasing method comprises the steps of: processing the second mixture or the liquefied sample-preserving solution I by using a nucleic acid releasing agent to obtain a released treatment solution; preferably, the nucleic acid releasing agent is selected from the nucleic acid releasing component in the viscous biological sample processing combination product of the third aspect of this disclosure;
preferably, a volume ratio of the second mixture or the liquefied sample-preserving solution 1 to the nucleic acid releasing component is 1:(0.5 to 1.5), further preferably 1:0.5, 1:0.6, 1:0.7, 1:0.8, 1:0.9, 1:1, 1:1.1, 1:1.2, 1:1.3, 1:1.4 or 1:1.5;
the extraction method comprises a step of: extracting nucleic acids in the released treatment solution with a nucleic acid extraction agent to obtain an extracted treatment solution;
the amplification method comprises a step of: amplifying the nucleic acids in the released treatment solution, the extracted treatment solution, the liquefied product or the liquefied sample-preserving solution II by using a nucleic acid amplification agent to obtain an amplification mixture;
the detection method comprises a step of: detecting the nucleic acids in the liquefied product, the liquefied sample-preserving solution II, the released treatment solution, the extracted treatment solution or the amplification mixture by using a nucleic acid detection agent;
the detection method is for non-diagnostic and non-therapeutic purposes, or the detection method is for one or both of diagnostic and therapeutic purposes.

In a ninth aspect of this disclosure, there is provided use of the viscous biological sample liquefying agent of the fourth aspect of this disclosure, or a combination product thereof, or a liquefaction kit thereof in the direct nucleic acid amplification or the direct detection,
preferably, the use is for non-diagnostic and non-therapeutic purposes;
wherein, the combination product comprises the viscous biological sample liquefying agent of the fourth aspect of this disclosure, and optionally a preservation component;
the liquefaction kit comprises the viscous biological sample liquefying agent of the fourth aspect of this disclosure.

In some embodiments, a real-time quantitative PCR instrument or a portable nucleic acid detector is used in combination.

Details of one or more examples of this disclosure are set forth in the following drawings and descriptions. Other features, objects and advantages of this disclosure will become apparent from the description, drawings and claims.

### BRIEF DESCRIPTION OF THE DRAWINGS

In order to more clearly explain the technical solution in the examples of this disclosure and understand this disclosure and its beneficial effects, the following will briefly introduce the drawings that need to be used in the descriptions of the examples. Obviously, the drawings in the following descriptions are merely some examples of this disclosure, for those having ordinary skills in the art, other drawings may also be obtained based on these drawings without making creative work.

FIG. 1A-1E show the amplification curves obtained from the rapid detection of human genome in groups in an example of this disclosure; FIG. 1A is the experimental group; FIG. 1B is protease method; FIG. 1C is sodium hydroxide method; FIG. 1D is DTT method; and FIG. 1E is acetylcysteine method;

FIG. 2A-2E show the amplification curves obtained from the respiratory syncytial virus detection in groups in an example of this disclosure; FIG. 2A is the experimental group; FIG. 2B is protease method; FIG. 2C is sodium hydroxide method; FIG. 2D is DTT method; and FIG. 2E is acetylcysteine method;

FIG. 3A-3B show the verification of the influence of the extraction free amplification system (the extracted nucleic acids into which the amplification is introduced is not purified) in an example of this disclosure; FIG. 3A shows the results of influence of the sample matrix No. 1 on the test amplification curve; FIG. 3B shows the results of influence of the sample matrix No. 2 on the test amplification curve;

FIG. 4A-4D show the sample stability test results after the liquefaction of reagents in an example of this disclosure; FIG. 4A is the RNA stability curve of the experimental group; FIG. 4B is the DNA stability curve of the experimental group; FIG. 4C is the RNA stability curve of the control group; and FIG. 4D is the DNA stability curve of the control group;

FIG. 5A-5D show the test results of respiratory virus after preservation in an example of this disclosure; FIG. 5A is sample No. 1, which is preserved with the example preservation component provided in this disclosure; FIG. 5B is sample No. 1, which is preserved with commercial preserving solution; FIG. 5C is sample No. 2, which is preserved with the example preservation component provided in this disclosure; and FIG. 5D is sample No. 2, which is preserved with commercial preserving solution;

FIG. 6A-6D show the test results of Neisseria gonorrhoeae after preservation in an example of this disclosure;

FIG. 6A is sample No. 1, which is preserved with the example preservation component provided in this disclosure; FIG. 6B is sample No. 1, which is preserved with commercial preserving solution; FIG. 6C is sample No. 2, which is preserved with the example preservation component provided in this disclosure; and FIG. 6D is sample No. 2, which is preserved with commercial preserving solution;

FIG. 7A-7D show the test results of cell samples after preservation in an example of this disclosure; FIG. 7A is sample No. 1, which is preserved with the example preservation component provided in this disclosure; FIG. 7B is sample No. 1, which is preserved with commercial preserving solution; FIG. 7C is sample No. 2, which is preserved with the example preservation component provided in this disclosure; and FIG. 7D is sample No. 2, which is preserved with commercial preserving solution;

FIG. 8A-8F show the test results of different samples after liquefaction and release in an example of this disclosure; FIG. 8A is the RNA amplification curve of respiratory virus No. 1; FIG. 8B is the RNA amplification curve of respiratory virus No. 2; FIG. 8C is the DNA amplification curve of human cells of sample No. 1; FIG. 8D is the DNA amplification curve of human cells of sample No. 2; FIG. 8E is the DNA amplification curve of Neisseria gonorrhoeae of sample No. 1; FIG. 8F is the DNA amplification curve of Neisseria gonorrhoeae of sample No. 2; and the Ct position corresponding to the reagent of this disclosure is marked in the figure;

FIG. 9A-9F show the test results of different samples after liquefaction, preservation and release in an example of this disclosure; FIG. 9A is the RNA amplification curve of respiratory virus No. 1; FIG. 9B is the RNA amplification curve of respiratory virus No. 2; FIG. 9C is the DNA amplification curve of human cells of sample No. 1; FIG. 9D is the DNA amplification curve of human cells of sample No. 2; FIG. 9E is the DNA amplification curve of Neisseria gonorrhoeae of sample No. 1; and FIG. 9F is the DNA amplification curve of Neisseria gonorrhoeae of sample No. 2;

FIG. 10A-10F show the test results of different samples after liquefaction and release in an example of this disclosure; FIG. 10A is the RNA amplification curve of respiratory virus No. 1; FIG. 10B is the RNA amplification curve of respiratory virus No. 2; FIG. 10C is the DNA amplification curve of human cells of sample No. 1; FIG. 10D is the DNA amplification curve of human cells of sample No. 2; FIG. 10E is the DNA amplification curve of Neisseria gonorrhoeae of sample No. 1; and FIG. 10F is the DNA amplification curve of Neisseria gonorrhoeae of sample No. 2;

FIG. 11A-11F show the test results of different samples after liquefaction, preservation and release in an example of this disclosure; FIG. 11A is the RNA amplification curve of respiratory virus No. 1; FIG. 11B is the RNA amplification curve of respiratory virus No. 2; FIG. 11C is the DNA amplification curve of human cells of sample No. 1; FIG. 11D is the DNA amplification curve of human cells of sample No. 2; FIG. 11E is the DNA amplification curve of Neisseria gonorrhoeae of sample No. 1; and FIG. 11F is the DNA amplification curve of Neisseria gonorrhoeae of sample No. 2;

FIG. 12A-12C show the test amplification curve of sputum samples containing respiratory virus (FIG. 12A), Neisseria gonorrhoeae (FIG. 12B) and human cells (FIG. 12C) using the release reagent (formulation 10-2) of an example of this disclosure;

FIG. 13A-13C show the test amplification curve of sputum samples containing respiratory virus (FIG. 13A), Neisseria gonorrhoeae (FIG. 13B) and human cells (FIG. 13C) using the release reagent (formulation 10-3) of an example of this disclosure;

FIG. 14A-14C show the test amplification curve of sputum samples containing respiratory virus (FIG. 14A), Neisseria gonorrhoeae (FIG. 14B) and human cells (FIG. 14C) using the release reagent (formulation 10-4) of an example of this disclosure;

FIG. 15A-15C show the test amplification curve of sputum samples containing respiratory virus (FIG. 15A), Neisseria gonorrhoeae (FIG. 15B) and human cells (FIG. 15C) using the release reagent (formulation 12-2) of an example of this disclosure;

FIG. 16A-16C show the test amplification curve of sputum samples containing respiratory virus (FIG. 16A), Neisseria gonorrhoeae (FIG. 16B) and human cells (FIG. 16C) using the release reagent (formulation 12-3) of an example of this disclosure;

FIG. 17A-17C show the test amplification curve of sputum samples containing respiratory virus (FIG. 17A), Neisseria gonorrhoeae (FIG. 17B) and human cells (FIG. 17C) using the release reagent (formulation 12-4) of an example of this disclosure;

FIG. 18A-18F show the detection results of respiratory viruses (FIG. 18A and FIG. 18B), Neisseria gonorrhoeae (FIG. 18C and FIG. 18D), and human cells (FIG. 18E and FIG. 18F) in an example of this disclosure; FIG. 18A, FIG. 18C and FIG. 18E are samples No. 1, which are subjected to liquefaction with the liquefying agent provided in this disclosure, PCR direct amplification and nucleic acid detection in sequence; FIG. 18B, FIG. 18D and FIG. 18F are samples No. 2, which are subjected to liquefaction with the liquefying agent in the control group, nucleic acid extraction, then PCR amplification and nucleic acid detection in sequence;

FIG. 19A-19C show the amplification curve of direct amplification after liquefaction with the liquefying agent in groups in example 16 of this disclosure, in which FIG. 19A corresponds to the amplification curve of respiratory virus, FIG. 19B corresponds to the DNA amplification curve of human cells, and FIG. 19C corresponds to the DNA amplification curve of Neisseria gonorrhoeae;

FIG. 20 shows the amplification curve in Example 18 of this disclosure, and the sample contains Neisseria gonorrhoeae.

### DETAILED DESCRIPTIONS OF THE EMBODIMENTS

This disclosure will be further described in details below in combination with the accompanying drawings, embodiments and examples. It should be understood that these embodiments and examples are only used to illustrate this disclosure and not to limit the scope of this disclosure. The purpose of providing these embodiments and examples is to make this disclosure more thorough and comprehensive. It should also be understood that this disclosure can be implemented in many different forms, and is not limited to the embodiments and examples described herein. Those skilled in the art can make various alterations or modifications without departing from the intensions of this disclosure, and the equivalent forms obtained also fall within the scope of protection of this disclosure. For example, the features explained or described as part of one embodiment may be combined in another embodiment in a suitable manner to produce a new embodiment. In addition, in the following description, a large number of specific details are given in order to provide a fuller understanding of this disclosure. It should be understood that this disclosure can be implemented without one or more of these details.

Unless otherwise defined, all terms used herein (including technical and scientific terms) have the same meanings as understood by those having ordinary skills in the art to which this application belongs. The terms used in the specification of this disclosure herein are only for the purpose of describing embodiments and examples, and are not intended to limit this disclosure.

### Terms

Unless otherwise stated or presence of a contradiction, the term or phrase used herein has the following meanings: the selection range of the terms “and/or”, “or/and”, and “as well as/or” as used herein includes any one of two or more related listed items, as well as any and all combinations of related listed items. The any and all combinations include any two related listed items, any more related listed items, or combinations of all related listed items. It should be noted that when at least three items are connected with a combination of at least two conjunctions selected from “and/or”, “or/and”, and “as well as/or”, it should be understood that in this disclosure, the technical solution undoubtedly includes the technical solution connected with “logic and” and the technical solution connected with “logic or”. For example, “A and/or B” includes three parallel solutions A, B and A+B. Yet for example, the technical solution of “A and/or B, and/or C, and/or D” includes any one of A, B, C, and D (that is, the technical solution connected by “logical or”), further includes any and all combinations of A, B, C, and D (that is, the combination of any two or any three of A, B, C, and D), and the combination of four of A, B, C, and D (that is, the technical solution connected by “logical and”).

In this disclosure, the description involving “multiple”, “many”, etc., means that the quantity is greater than or equal to 2, unless otherwise specified. For example, “one or more” is equal to 1 or greater than or equal to 2 in quantity, which can be one, two or more.

The phases “combination thereof”, “any combination thereof”, “any combination mode thereof”, etc., as used herein include all appropriate combination modes of any two or more items in the listed items.

The “appropriate” in “appropriate combination mode”, “appropriate mode”, “any appropriate mode”, etc., as used herein is used in a way that enables to implement the technical solution of this application, solve the technical problems of this application, and achieve the expected technical effect of this application.

Terms “preferably”, “more preferably”, “better”, “appropriate”, “further preferably”, etc., as used herein are only to describe better embodiments or examples, it should be understood that they do not constitute a limitation on the protection scope of this disclosure. If “preferably” appears in multiple places in a technical solution, each “preferably” is independent of each other, unless otherwise specified, and there are no contradictions or mutual constraints. In a technical solution, when “preferably” and one or more “further preferably” appear at the same time, any two or any more “preferably” may not be combined, or they may be combined to form different features.

In this disclosure, “further”, “still further”, “particularly”, etc., are used for descriptive purposes, indicating differences in content, but should not be understood as restrictions on the protection scope of this disclosure.

In this disclosure, “optionally” can be none or arbitrary selection, unless otherwise defined.

In this disclosure, “optionally”, “optional”, “option” means that an element may or may not be needed, that is, any one of the two parallel solutions selected from “with” or “without”. If “optionally” appears in multiple sections in a technical solution, each “optionally” is independent of each other, unless otherwise specified, and there are no contradictions or mutual constraints. In this disclosure, descriptions such as “optionally contain”, “optionally comprise” etc., refer to “containing or without containing”. “Optional component X” means that component X is present or is absent.

In this disclosure, in the “first aspect”, “second aspect”, “third aspect”, “fourth aspect”, “fifth aspect”, “sixth aspect”, etc., the terms “first”, “second”, “third”, “fourth” and so on are only used for descriptive purposes to distinguish between similar things or features, and cannot be understood as indicating or implying relative importance or quantity, nor can it be understood as implicitly indicating the importance or quantity of the indicated technical features. Moreover, “first”, “second”, “third”, “fourth” and so on only serve the purpose of non-exhaustive enumeration and description, and it should be understood that they do not constitute a closed limit on quantity.

Terms “contain”, “include” and “comprise” as used in this disclosure are synonyms, which are inclusive or open-ended, and do not exclude additional, unreferenced members, elements or method steps.

In this disclosure, the technical features described in an open-ended manner include the closed-ended technical solution composed of the listed features, as well as the open-ended technical solution containing the listed features. For example, a viscous biological sample liquefying agent comprising the following components: guaifenesin, a first strong base, a first inorganic cation (e.g., Na⁺, K⁺ or a combination thereof), Surfactin and a first aqueous solvent, comprises both the liquefying agent is consisted of guaifenesin, the first strong base, the first inorganic cation, Surfactin and the first aqueous solvent (a closed-ended solution) and the open-ended solution that further allows the addition of other components.

The numerical ranges represented by endpoints in this disclosure include all numerical values and fractions contained in the ranges, as well as the quoted endpoints.

In this disclosure, the numerical intervals (i.e., numerical ranges) are involved. Unless otherwise specified, the optional numerical values are regarded as continuous when distributed within the above numerical ranges, and include the two numerical endpoints of the numerical ranges (i.e., the minimum and maximum values), as well as each numerical value between the two numerical endpoints. The “numerical value” in the numerical interval can be any quantitative value, such as number, percentage, proportion, etc. The “numerical interval” allows to broadly include quantitative intervals such as percentage interval, proportion interval and ratio interval. Unless otherwise specified, when the numerical interval only refers to the integers within the numerical interval, it includes the two endpoint integers of the numerical range and each integer between the two endpoints. In addition, when multiple ranges are provided to describe features or characteristics, these ranges can be combined. In other words, unless otherwise specified, the scope disclosed herein shall be understood to include any and all subranges included therein.

In this disclosure, the unit involved in the data range only having a unit behind the right endpoint means that the units of the left endpoint and the right endpoint are the same. For example, 1-200 μM means that the units of left endpoint 1 and right endpoint 200 are both in microns.

The concentration value involved in this disclosure includes fluctuations within a certain range. For example, it can fluctuate within the corresponding accuracy range. For example, 2% can be allowed to fluctuate within ±0.1%. For larger values or values that need no fine control, it is also allowed to include greater fluctuations. For example, 100 mM can be allowed to fluctuate within the range of ±1%, ±2%, ±5%, etc. When a molecular weight is involved, it is allowed to include fluctuations within ±10%.

In this disclosure, the term “room temperature” generally refers to 4° C.-35° C., preferably 20° C.±5° C. In some embodiments of this disclosure, room temperature refers to 20° C.-30° C.

In this disclosure, m/v refers to the mass volume ratio, and % (m/v) refers to the mass percentage contained in a certain volume of mixed system. For example, A substance A in the mixed system having a concentration of 5% (m/v) means that there are 5 g of substance A per 100 ml of the mixed system.

In this disclosure, both mmol/L and mM refer to millimoles per liter, and can be used interchangeably. Both mol/L and M refer to moles per liter and can be used interchangeably.

In this disclosure, taking “within 15 min” as an example, it refers to less than or equal to 15 min, unless otherwise specified.

All documents mentioned in the present application are cited as references in the present disclosure, just as each document is individually incorporated as a reference. Unless it is in conflict with the inventive purpose, technical solution or both of the disclosure, the references involved in the disclosure are incorporated in all contents and purposes. When the cited documents are involved in this disclosure, the definitions of relevant technical features, terms, nouns, phrases, etc. in the cited documents are also incorporated. When the references are involves in this disclosure, examples of the cited relevant technical features and the preferred modes can also be incorporated into this disclosure as a reference, but only to the extent that this disclosure can be implemented. It should be understood that when the cited content conflicts with the description in this disclosure, this disclosure shall prevail or the cited content shall be amended adaptively according to the description of this disclosure.

In this disclosure, in “the first aqueous solvent” (firstly defined in the liquefying agent of the second aspect), “the second aqueous solvent” (in nucleic acid releasing component i)), “the fourth aqueous solvent” (firstly defined in the viscous biological sample liquefying agent of the fourth aspect), “the first buffer component” (firstly defined in the liquefying agent of the second aspect), “the second buffer component” (in nucleic acid releasing component i)), “the third buffer component” (in nucleic acid releasing component ii)), “the fourth buffer component” (in the viscous biological sample liquefying agent in the fourth aspect), “the second inorganic cation” (in nucleic acid releasing component i)), “the third inorganic cation” (in nucleic acid releasing component ii)), “the first strong base” (in the viscous biological sample liquefying composition of the first aspect), “the second strong base” (in nucleic acid releasing component i)), “the third strong base” (in nucleic acid releasing component ii)), “the first adsorbing agent” (in nucleic acid releasing component i)), “the second adsorbing agent” (in nucleic acid releasing component ii)), “the third adsorbing agent” (in the liquefaction component of the viscous biological sample processing combination product of the third aspect), “the fourth adsorbing agent” (in the viscous biological sample liquefying agent of the fourth aspect), “the first mixture” (in the liquefaction method of the seventh aspect), “the second mixture” (in the liquefaction method of the seventh aspect), etc, the terms “first”, “second”, “third” and so on are only used for the purpose of distinguishing description, and cannot be understood as indicating or implying the relative importance or quantity, nor as implying the importance or quantity of the indicated technical features. Moreover, “first”, “second”, “third”, and so on only serve the purpose of non-exhaustive enumeration and description, and it should be understood that they do not constitute a closed limit on quantity. For example, in this disclosure, the first strong base, the second strong base and the third strong base are all strong bases, which can be the same or different. “First”, “second” and “third” are only used as distinguishing markers to distinguish strong bases used in different embodiments. In this disclosure, the molar concentration of strong base refers to the concentration of hydroxyl ions as provided, unless otherwise specified. When defining specific strong bases, such as the molar concentration of sodium hydroxide, the specific definition shall prevail. For example, 1 M strong base refers to the ability to provide 1 M hydroxyl ions, 1 M sodium hydroxide or other kinds of strong base means that the molar concentration of the strong base is 1 M, and if one molecule of strong base carries more than one hydroxyl ions, the ability to provide the molar concentration of hydroxyl ions may exceed 1 M.

In this disclosure, in the “first mixture”, “second mixture”, etc., the terms “first”, “second”, etc. are only used for the purpose of distinguishing description, and cannot be understood as indicating or implying the relative importance or quantity, nor as implying the importance or quantity of the indicated technical features. Moreover, “first”, “second”, and so on only serve the purpose of non-exhaustive enumeration and description, and it should be understood that they do not constitute a closed limit on quantity.

The “water” involved in this disclosure can be each independently distilled water, purified water, filtered water, deionized water, etc; each independently preferably nucleic acid free water, and each independently preferably nuclease free water.

In this disclosure, the concentration or dosage of each component in the liquefying agent (including but not limited to the liquefying agent of the second aspect and the viscous biological sample liquefying agent of the fourth aspect) refers to the final concentration or final dosage in the liquefying agent, unless otherwise defined.

In this disclosure, the concentration or dosage of each component in the liquefaction component refers to the final concentration or final dosage in the liquefaction component, which is equivalent to the final concentration or final dosage in the liquefaction component constituting the liquefying reagent, unless otherwise defined.

In this disclosure, the concentration or dosage of each component in the nucleic acid releasing component refers to the final concentration or final dosage in the nucleic acid releasing component, which is equivalent to the final concentration or final dosage in the nucleic acid releasing component constituting the release reagent, unless otherwise defined.

In this disclosure, the concentration or dosage of each component in the preservation component refers to the final concentration or final dosage in the preservation component, which is equivalent to the final concentration or final dosage in the preservation component constituting the preservation agent, unless otherwise defined. For example, the inorganic cation component in the preservation component provided.

In addition, sputum samples with great viscosity will easily leads to the problem of uneven mixing in the process of conventional liquefaction. Nucleic acids (especially RNAs) in sputum samples are extremely unstable and are usually degraded in a few hours under greenhouse conditions. However, in clinical testing, nucleic acids in sputum samples are often not processed and detected in time. Therefore, it is necessary to develop a method that can quickly and fully liquefy sputum and effectively protect sample nucleic acids at the same time. At present, there are many published inventions about sputum liquefaction and nucleic acid protection. However, these methods generally cause problems such as complex composition, high cost, complex operation, insufficient sample mixing and liquefaction, etc.

At present, there are many published inventions about sputum liquefaction and nucleic acid protection. However, these methods generally cause problems such as complex composition, high cost, complex operation, insufficient sample mixing and liquefaction, etc., and how to effectively preserve the liquefied samples is also a problem that needs to be solved in this field. In addition, how to efficiently release nucleic acids from liquefied samples is also a problem to be solved in this field.

In addition, due to the sputum samples with great viscosity and many interfering components, when nucleic acid detection is carried out in traditional ways, after liquefaction, the samples still need to be subjected to complex sample processing processes such as releasing, purifying, etc., for removal of the factors that inhibit the PCR reaction, and then to be subjected to amplification, detection of nucleic acids, or both. This leads to cumbersome steps, complex operations, time-consuming and high cost during the process from the sample collection to the sample detection, which is not suitable for mass processing of sputum samples clinically.

In a first aspect of this disclosure, there is provided a viscous biological sample liquefying composition comprising guaifenesin and a strong base (denoted as a first strong base).

In this disclosure, the terms “biological sample”, “sample” and so on refer to animal samples, which may derive from: tissue, organs, tissue lysates in animals (preferably including at least mammals, such as primates, including humans); cells (in the subject, directly obtained from the subject, or maintained in the culture or from the cells of the cultured cell lines), cell lysates (or lysate part) or cell extracts; a solution containing one or more molecules derived from cells, cellular materials or viral materials (such as polypeptides or nucleic acids); or a solution containing naturally occurring or non-naturally occurring nucleic acids, which can be determined by or as described herein. In some embodiments, the sample contains nucleic acids. The sample can also be any body fluid or excretion containing one or more cells, cell components or nucleic acids, including but not limited to cells, nuclear or acellular nucleic acids. In particular, the biological sample of this disclosure preferably derives from body fluid, which includes liquid, semi-solid, aerated liquid, liquid-gas mixture, etc., derived from animals. Such body fluids may include, but are not limited to, saliva, sputum, serum, plasma, blood, urine, mucus, sweat, tears or other eye fluids, ear fluids, face (e.g., from blisters or sores), gastric fluid or gastric juice, fecal fluid, pancreatic fluid or juice, seminal fluid, lactation or assay products, spinal fluid, liquid bone marrow or lymph fluid.

“Viscous biological sample” refers to a biological sample with viscosity, especially a viscous body fluid. Viscosity can be caused by a large number of mucins and polysaccharides (especially mucopolysaccharides) or proteoglycans, etc., present in the biological samples. A preferred viscous biological sample is sputum, cervical mucus or a combination thereof. Examples of the viscous biological sample are nasopharyngeal swabs, oral swabs, lavage fluids, etc. “Mucin” refers to any mucoproteins that increase the viscosity of the cytoplasmic matrix around secretory cells. “Sputum” refers to viscous substances contained in or discharged from the nasal cavity or oral cavity of mammals (usually from the respiratory tract).

In some embodiments, the viscous biological sample is sputum, cervical mucus, nasopharyngeal swab or oral swab.

In this disclosure, “composition” may refer to a mixture or two or more substances that can be provided or used in combination. Therefore, in the viscous biological sample liquefying composition, guaifenesin and the first strong base can be mixed together or packaged separately.

Guaifenesin and the first strong base in the viscous biological sample liquefying composition are within the range of liquefaction components, and the viscous biological sample liquefying composition can be used to formulate a liquefying agent.

In this disclosure, “liquefier” and “liquefying agent”, which have the same meaning and can be used interchangeably, are a mixture composed of liquefaction components and serving as an independent system. The different components in the “liquefaction component” can be independent agents or non-independent components. The liquefaction component is for the degradation of a viscous substance in the viscous biological sample species, reducing the viscosity of the sample and making the subsequent operations easy. The liquefaction component used in this disclosure can be liquefied at room temperature (such as 20-30° C.) and without harsh operating conditions, which does not affect the subsequent nucleic acid detection, and is compatible with the extraction free amplification system, and has no obvious adverse effect on the preservation time of nucleic acids in the sample.

The viscous biological sample liquefying composition of this disclosure uses a specific combination of guaifenesin and strong base, which has excellent liquefaction effect compared with traditional liquefying agents. Reference can be made to example 1 of this disclosure.

In this disclosure, with the first strong base, pH value of the prepared liquefying agent can be adjusted to be greater than or equal to 10, which may be 10-14, further may be 10-12 or 12-14, and still further may be 10, 10.5, 11, 11.5, 12, 12.5, 13, 13.5 or 14.

In the viscous biological sample liquefying composition of this disclosure, the first strong base is used to provide a strong alkali environment, which is used to provide a strong alkali environment with a pH greater than or equal to 10. There is no special limit on the type of the first strong base, provided that it can provide a pH condition that is sufficient for liquefaction, and has no adverse effect on the subsequent PCR.

In this disclosure, the term “strong base” refers to a substance from which all anions ionized in an aqueous solution are hydroxyl ions. It can be organic strong base or inorganic strong base, including but not limited to at least one of lithium hydroxide, sodium hydroxide, potassium hydroxide, rubidium hydroxide, francium hydroxide, cesium hydroxide, calcium hydroxide, choline, silver hydroxide, thallium hydroxide, quaternary ammonium base, strontium hydroxide, barium hydroxide, radium hydroxide, silver diamminohydroxide, etc. In some embodiments, the strong base is at least one of sodium hydroxide, potassium hydroxide, lithium hydroxide, calcium hydroxide, barium hydroxide or choline. In some preferred embodiments, the strong base is sodium hydroxide and/or potassium hydroxide, i.e., sodium hydroxide, potassium hydroxide or a combination thereof.

In some embodiments, the first strong base is at least one of sodium hydroxide, potassium hydroxide, lithium hydroxide, calcium hydroxide, barium hydroxide or choline.

In some preferred embodiments, the first strong base is sodium hydroxide, potassium hydroxide or a combination thereof.

In some embodiments, the viscous biological sample liquefying composition further includes rigid microparticles. The viscous biological sample liquefying composition also optionally includes rigid microparticles. That is, rigid microparticles can be used as optional components.

In some embodiments, the viscous biological sample liquefying composition further includes rigid microparticles, which can accelerate the mixing.

In some embodiments, rigid microparticles correspond to a separate package.

In this disclosure, the material of rigid microparticles can be any hard material. The term “rigid” refers to that it usually does not cause obvious damage when mixing/assisting in mixing a viscous biological sample, and does not release components that interfere with subsequent processes (such as at least one process in the preservation, release, enrichment, amplification and detection of nucleic acids). Its materials include but are not limited to metals (which can be a single substance or alloys) or metal oxides, ceramics, glass, hard plastics, natural or artificial mineral components, etc. In some preferred embodiments, the material of the rigid microparticles includes at least one of zirconia, silicon nitride, ceramsite, hard stainless steel, hard tungsten carbide, sintered corundum or agate.

Rigid microparticles can be used to assist in mixing the viscous biological sample and the viscous biological sample liquefying composition, reduce the processing time and increase the processing efficiency.

In this disclosure, the term “microparticles” can be a sphere, a near sphere, an ellipsoid, a column, a rod, a polyhedron (such as a cube for example) or an irregularly shape, preferably microsphere. The average particle size of microparticles is preferably in millimeter-sized, for example, 0.01 mm-500 mm, further may be 0.05 mm, 0.1 mm, 0.2 mm, 0.3 mm, 0.4 mm, 0.5 mm, 1 mm, 2 mm, 3 mm, 4 mm, 5 mm, 6 mm, 7 mm, 8 mm, 9 mm, 10 mm, 20 mm, 30 mm, 40 mm, 50 mm, 100 mm, 200 mm, 300 mm, 400 mm, 500 mm, etc., and further preferably 0.01 mm-10 mm.

In some embodiments of this disclosure, the rigid microparticles are of a shape including one or more of sphere, near sphere, ellipsoid, column, rod, polyhedron and irregular shape; in some of these embodiments, rigid microparticles are spheres, near spheres, ellipsoids, columns, rods, polyhedrons, or irregular shapes.

In other embodiments of this disclosure, the rigid microparticles are of a shape including one or more of sphere, cube, polyhedron and irregular shape. In some of these embodiments, rigid microparticles are in a shape of spheres, cubes, polyhedrons, or irregular shapes.

In some embodiments of this disclosure, the average particle size of the rigid microparticles may be 0.01 mm-500 mm; further may be 0.05 mm-500 mm; further may be 0.1 mm-500 mm; further may be 0.2 mm-500 mm; further may be 0.5 mm-500 mm; further may be 1 mm-500 mm; further may be 0.01 mm-200 mm; further may be 0.05 mm-200 mm; further may be 0.1 mm-200 mm; further may be 0.2 mm-200 mm; further may be 0.5 mm-200 mm; further may be 1 mm-200 mm; further may be 0.01 mm-100 mm; further may be 0.05 mm-100 mm; further may be 0.1 mm-100 mm; further may be 0.2 mm-100 mm; further may be 0.5 mm-100 mm; further may be 1 mm-100 mm; further may be 0.01 mm-50 mm; further may be 0.05 mm-50 mm; further may be 0.1 mm-50 mm; further may be 0.2 mm-50 mm; further may be 0.5 mm-50 mm; further may be 1 mm-50 mm; further may be 0.01 mm-10 mm; further may be 0.05 mm-10 mm; further may be 0.1 mm-10 mm; further may be 0.2 mm-10 mm; further may be 0.5 mm-10 mm; further may be 1 mm-10 mm; further may be 0.01 mm-5 mm; further may be 0.05 mm-5 mm; further may be 0.1 mm-5 mm; further may be 0.2 mm-5 mm; further may be 0.5 mm-5 mm; further may be 1 mm-5 mm. The range between the preceding examples allows for appropriate combinations.

In some embodiments of this disclosure, the dosage of rigid microparticles is 0.1 g/mL-2 g/mL. Examples of dosage include but are not limited to 0.1 g/mL, 0.2 g/mL, 0.3 g/mL, 0.4 g/mL, 0.5 g/mL, 0.6 g/mL, 0.7 g/mL, 0.8 g/mL, 0.9 g/mL, 1.0 g/mL, 1.1 g/mL, 1.2 g/mL, 1.3 g/mL, 1.4 g/mL, 1.5 g/mL, 1.6 g/mL, 1.7 g/mL, 1.8 g/mL, 1.9 g/mL, 2.0 g/mL, etc.

In this disclosure, “dosage of rigid microparticles” refers to the final dosage in the liquefaction component, or refers to the final dosage in the liquefying agent prepared from the viscous biological sample liquefying composition, unless otherwise defined.

In some embodiments of this disclosure, the dosage of rigid microparticles is 0.1 g/mL-2 g/mL; further may be 0.2 g/mL-2 g/mL; further may be 0.5 g/mL-2 g/mL; further may be 0.8 g/mL-2 g/mL; further may be 1 g/mL-2 g/mL; further may be 0.1 g/mL-1.5 g/mL; further may be 0.2 g/mL-1.5 g/mL; further may be 0.5 g/mL-1.5 g/mL; further may be 0.8 g/mL-1.5 g/mL; further may be 1 g/mL-1.5 g/mL; further may be 0.1 g/mL-1.2 g/mL; further may be 0.2 g/mL-1.2 g/mL; further may be 0.5 g/mL-1.2 g/mL; further may be 0.8 g/mL-1.2 g/mL; further may be 1 g/mL-1.2 g/mL; further may be 0.1 g/mL-1 g/mL; further may be 0.2 g/mL-1.5 g/mL; further may be 0.5 g/mL-1 g/mL; further may be 0.8 g/mL-1 g/mL. The range between the preceding examples allows for appropriate combinations.

In some embodiments of this disclosure, the viscous biological sample liquefying composition includes guaifenesin, strong base and rigid microparticles. In some preferred embodiments, guaifenesin, strong base and rigid microparticles are provided in a mixed system containing the three (collectively packaged in one container together), and preferably further include a solvent, which is further preferably an aqueous solvent, more preferably water or buffer solution. Among them, the content of guaifenesin can be preferably 1 mmol/L-1 mol/L, the content of strong base can be preferably 5 mmol/L-500 mmol/L (further preferably sodium hydroxide), and the content of rigid microparticles can be preferably 0.1 g/mL-2 g/mL (further preferably zirconia beads).

In some preferred embodiments of this disclosure, the viscous biological sample liquefying composition is consisted of guaifenesin, strong base, rigid microparticles and aqueous solvent.

The components in the viscous biological sample liquefying composition can be packaged separately or in combination.

In some embodiments of this disclosure, the viscous biological sample liquefying composition is a composition packaged in one container. In some embodiments, guaifenesin and strong base in the viscous biological sample liquefying composition are contained in one packaging container, and rigid microparticles in another packaging container. In some embodiments, guaifenesin, strong base, and rigid microparticles are individually packaged separately.

The viscous biological sample liquefying composition can be a solid (preferably a dry powdery solid) or a liquid, or a state of being somewhere in between, such as a gelatinous state.

In some embodiments, the liquefying composition is a solution. The solvent of the solution can be water, for example, distilled water, purified water, filtered water, deionized water, etc; preferably nucleic acid free water, and more preferably nuclease free water.

In some embodiments, the liquefying composition is a solution, and further, the concentration of the rigid microparticles is 0.1 g/ml-2.0 g/ml, for example 0.2 g/ml, 0.3 g/ml, 0.4 g/ml, 0.5 g/ml, 0.6 g/ml, 0.7 g/ml, 0.8 g/ml, 0.9 g/ml, 1.0 g/ml, 1.1 g/ml, 1.2 g/ml, 1.3 g/ml, 1.4 g/ml, 1.5 g/ml, 1.6 g/ml, 1.7 g/ml, 1.8 g/ml or 1.9 g/ml.

In a second aspect of this disclosure, there is provided a liquefying agent, which includes the viscous biological sample liquefying composition as described in the first aspect of this disclosure.

The definition of “liquefying agent” is consistent with the foregoing.

After mixing guaifenesin with the first strong base, additional components can be added as appropriate (non-limiting examples of additional components are one or more of an aqueous solvent, an adsorbing agent, rigid microparticles, etc., for example), to obtain the liquefying agent for liquefying the viscous biological sample.

In some embodiments of this disclosure, in the liquefying agent, the concentration of guaifenesin is 1 mmol/L-1 mol/L, the concentration of the first strong base is less than 1 mol/L, and further can be 0.1 mmol/L-500 mmol/L. In some embodiments of this disclosure, the liquefying agent is prepared by mixing the viscous biological sample liquefying composition as described in the first aspect of this disclosure.

In this disclosure, when using the liquefying agent to process the viscous biological sample, it needs to be carried out in an aqueous environment, thus it is necessary to provide a solvent, and preferably an aqueous solvent, unless otherwise specified. However, it should be understood that the solvent can be additionally provided during the operation. Therefore, for the liquefaction component, the solvent is optional; and for various liquefying agents in this disclosure, solvents are usually included, unless otherwise specified.

In some embodiments of this disclosure, the liquefaction component preferably further includes an aqueous solvent, which is further preferably water or a buffer solution.

In some embodiments of this disclosure, the liquefying agent in the second aspect of this disclosure further includes an aqueous solvent, which further can be water or a buffer solution.

In some embodiments of this disclosure, the concentration of guaifenesin is 1 mmol/L-1 mol/L. Here, as for the concentration of guaifenesin, it refers to the concentration in the liquefying agent, unless otherwise specified.

In some embodiments of this disclosure, the concentration of guaifenesin is 20 mmol/L-500 mmol/L.

In some embodiments of this disclosure, the concentration of guaifenesin is in a range including but being not limited to: 20 mmol/L-400 mmol/L, 50 mmol/L-200 mmol/L, 50 mmol/L-150 mmol/L, etc.

In some embodiments of this disclosure, examples of the concentration of guaifenesin include, but are not limited to: 20 mmol/L, 40 mmol/L, 50 mmol/L, 60 mmol/L, 80 mmol/L, 100 mmol/L, 120 mmol/L, 140 mmol/L, 150 mmol/L, 160 mmol/L, 180 mmol/L, 200 mmol/L, 220 mmol/L, 240 mmol/L, 250 mmol/L, 260 mmol/L, 280 mmol/L, 300 mmol/L, 320 mmol/L, 340 mmol/L, 350 mmol/L, 360 mmol/L, 380 mmol/L, 400 mmol/L, 420 mmol/L, 450 mmol/L, 500 mmol/L, etc.

In some embodiments, the concentration of guaifenesin in the liquefying agent may be 1 mmol/L-1 mol/L; further may be 20 mmol/L-1 mol/L; further may be 20 mmol/L-500 mmol/L; further may be 20 mmol/L-400 mmol/L; further may be 20 mmol/L-200 mmol/L; further may be 20 mmol/L-150 mmol/L; further may be 50 mmol/L-400 mmol/L; further may be 50 mmol/L-200 mmol/L; further may be 50 mmol/L-150 mmol/L; further may be 50 mmol/L, 100 mmol/L, 150 mmol/L or 200 mmol/L. The range between the preceding examples allows for appropriate combinations.

In some embodiments of this disclosure, the first strong base is a monobasic base in the liquefying agent, which is used to provide a pH environment with pH greater than or equal to 10. In some embodiments, the concentration of the first strong base in the liquefying agent is less than 1 mol/L, and further may be less than or equal to 500 mmol/L. In some embodiments, the concentration of the first strong base in the liquefying agent is 0.1 mmol/L-500 mmol/L, further may be 1 mmol/L-500 mmol/L. In some embodiments, the concentration of the first strong base in the liquefying agent is 5 mmol/L-500 mmol/L. Here, the concentration of the first strong base refers to the concentration in the liquefying agent, unless otherwise specified. Examples of the concentration range of the first strong base include but are not limited to 0.1 mmol/L-500 mmol/L, 1 mmol/L-500 mmol/L, 5 mmol/L-500 mmol/L, 5 mmol/L-400 mmol/L, 5 mmol/L-200 mmol/L, 5 mmol/L-100 mmol/L, 5 mmol/L-80 mmol/L, 5 mmol/L-60 mmol/L, 5 mmol/L-50 mmol/L, 5 mmol/L-40 mmol/L, 10 mmol/L-200 mmol/L, 10 mmol/L-100 mmol/L, 10 mmol/L-50 mmol/L, 10 mmol/L-40 mmol/L, etc. Examples of concentrations include, but are not limited to 0.1 mmol/L, 0.5 mmol/L, 0.6 mmol/L, 0.8 mmol/L, 1 mmol/L, 2 mmol/L, 3 mmol/L, 4 mmol/L, 4.5 mmol/L, 5 mmol/L, 6 mmol/L, 7 mmol/L, 8 mmol/L, 9 mmol/L, 10 mmol/L, 12 mmol/L, 15 mmol/L, 20 mmol/L, 25 mmol/L, 30 mmol/L, 40 mmol/L, 50 mmol/L, 60 mmol/L, 70 mmol/L, 80 mmol/L, 90 mmol/L, 100 mmol/L, 120 mmol/L, 140 mmol/L, 150 mmol/L, 200 mmol/L, 250 mmol/L, 300 mmol/L, 350 mmol/L, 400 mmol/L, 450 mmol/L, 500 mmol/L, etc. The technical features in this embodiment can be combined in other embodiments in a suitable manner.

In some embodiments of this disclosure, the first strong base in the liquefying agent is at least one of sodium hydroxide, potassium hydroxide, lithium hydroxide, calcium hydroxide, barium hydroxide, choline, etc.

In some embodiments of this disclosure, the first strong base in the liquefying agent is sodium hydroxide and/or potassium hydroxide (i.e., sodium hydroxide, potassium hydroxide or a combination thereof). Further, the concentration of the first strong base is less than 1 mol/L, may be 0.1 mmol/L-500 mmol/L, further may be 1 mmol/L-500 mmol/L, still further may be 5 mmol/L-500 mmol/L. Examples of concentration range and specific concentration can refer to the examples of monobasic base above, and the combined features can be formed in a suitable manner.

In some embodiments, the first strong base in the liquefying agent is sodium hydroxide.

In some embodiments, the concentration of the first strong base in the liquefying agent may be 1 mmol/L-500 mmol/L; further may be 5 mmol/L-500 mmol/L; further may be 5 mmol/L-400 mmol/L; further may be 5 mmol/L-200 mmol/L; further may be 5 mmol/L-100 mmol/L; further may be 5 mmol/L-80 mmol/L; further may be 5 mmol/L-60 mmol/L; further may be 5 mmol/L-50 mmol/L; further may be 5 mmol/L-40 mmol/L; further may be 10 mmol/L-200 mmol/L; further may be 10 mmol/L-100 mmol/L; further may be 10 mmol/L-50 mmol/L; further may be 10 mmol/L-40 mmol/L; further may be 40 mmol/L, 50 mmol/L, 60 mmol/L, 80 mmol/L, or 100 mmol/L. The range between the preceding examples allows for appropriate combinations.

In some embodiments, the pH value of the liquefying agent is greater than or equal to 10, may be 10-14, further may be 10-12 or 12-14, further may be 10, 10.5, 11, 11.5, 12, 12.5, 13, 13.5 or 14.

In some embodiments, guaifenesin and the first strong base in the liquefying agent are provided in the form of a composition (collectively packaged in one container). At this point, the concentration of guaifenesin in the liquefying agent can be preferably selected from 1 mmol/L-1 mol/L, and the concentration of the first strong base in the liquefying agent can be preferably selected from 5 mmol/L-500 mmol/L (further preferably sodium hydroxide). The concentration of guaifenesin and the first strong base can be selected from the examples of concentration or concentration range as recorded herein. The concentration between the two can be combined in a suitable manner to achieve a better liquefaction effect.

In some preferred embodiments, the liquefying agent prepared by the liquefying composition of the first aspect of this disclosure includes 100 mmol/L of guaifenesin and 10 mmol/L of sodium hydroxide.

In some embodiments, in the liquefying agent of the second aspect of this disclosure, the concentration of guaifenesin is 20 mmol/L-500 mmol/L, the concentration of the first strong base is less than 1 mol/L (preferably 0.1 mmol/L-500 mmol/L, further preferably 1 mmol/L-500 mmol/L, still further preferably 5 mmol/L-500 mmol/L). Further, the concentration of guaifenesin and the concentration of the first strong base further can be selected from any of the above suitable concentrations independently or in combination with each other.

The liquefying agent of the second aspect of this disclosure is prepared by the corresponding liquefaction component. The liquefaction component optionally includes a solvent; when a solvent is included, the solvent is preferably an aqueous solvent; and the aqueous solvent here is denoted as the first aqueous solvent. In some preferred embodiments, the liquefaction component is consisted of guaifenesin, a strong base (denoted as the first strong base) and a solvent (further preferably an aqueous solvent), that is, at this point, the liquefying agent in the second aspect of this disclosure is consisted of guaifenesin, a first strong base and a solvent (further preferably an aqueous solvent).

For example, the term “aqueous solvent” as used herein refers to a solvent or solution containing water, and may be a single solvent consisted of pure water, or may be a mixed solvent formed by the miscibility of water and other solvents. Solvents that can be miscible with water include but are not limited to alcohol solvents (such as methanol, ethanol, propanol, isopropanol, polyethylene glycol, etc.). The aqueous solvent may further contain salt components.

In some embodiments of this disclosure, the first aqueous solvent is water, for example, distilled water, purified water, filtered water, deionized water, etc; preferably nucleic acid free water, more preferably nuclease free water. In some embodiments of this disclosure, the first aqueous solvent is a buffer component, which is denoted as the first buffer component.

As used herein, the term “buffer solution”, also referred to herein as “buffer component”, refers to an aqueous solution or a composition that resists changes in pH when an acid or a base is added to the solution or composition. This resistance to pH changes is due to the buffering properties of such liquids. Therefore, a solution or composition showing buffering activity is called as a buffer or buffer solution. Buffer generally does not have unlimited ability to maintain the pH of a solution or composition. On the contrary, with a buffer, a pH may be generally kept within a specific range, for example, pH 6-pH 8, or pH greater than or equal to 10, for example. Generally, with the buffer, a pH may be kept within logarithm of its pKa±1 (see, for example, Mohan, Buffers, A guide for the preparation and use of buffers in biological systems, CALBIOCHEM, 1999). When the liquefaction component includes a buffer component, the buffer component (i.e., the first buffer component) is designed to maintain the pH of the liquefaction system in a strong alkali environment, such as pH greater than or equal to 10. The components of the buffer components herein can be packaged independently in one or more containers. When its components are packaged independently in one container, it can be used as a prefabricated buffer reagent.

The liquefaction component for preparing the liquefying agent of the second aspect of this disclosure optionally includes an adsorbing agent, that is, the adsorbing agent is an optional component in the liquefying agent of the second aspect of this disclosure and is unessential.

In this disclosure, the meaning of “the liquefaction component for preparing the liquefying agent of the second aspect of this disclosure” means that the liquefying agent as described in the second aspect of this disclosure is consisted of the liquefaction component.

In some embodiments, the liquefaction component for preparing the liquefying agent of the second aspect of this disclosure further includes an adsorbing agent, which is denoted as the third adsorbing agent.

The “adsorbing agent” as used herein can adsorb impurities through physical or chemical action to reduce the interference of impurities to subsequent detections. The adsorbing agent can be of any suitable type under the principle of not adversely affecting liquefaction. The chemical actions for adsorbing impurities include but are not limited to chelation. The adsorbing agent may be resin or chelate, or cheating resin. In some embodiments, the adsorbing agent is resin. In some preferred embodiments, the adsorbing agent is resins such as polypropylethylenes, polyacrylic acids, polyvinyl alcohols, chitosans, etc, further preferably chelating resins. In some embodiments, the adsorbing agent is Chelex resin. In some embodiments, the concentration of the adsorbing agent in the liquefying agent is 1%-15% (w/v), for example, 0.5%, 1%, 1.5%, 2%, 3%, 4%, 5%, 6%, 7%, 8%, 9%, 10%, 11%, 12%, 13%, 14%, 15%, etc.

In some preferred embodiments, the third adsorbing agent is chelating resin, and further is Chelex resin. In some embodiments, the concentration of the third adsorbing agent in the liquefaction component is 1%-15% (w/v), for example, 0.5%, 1%, 1.5%, 2%, 3%, 4%, 5%, 6%, 7%, 8%, 9%, 10%, 11%, 12%, 13%, 14%, 15%, etc., wherein, the third adsorbing agent may be preferably a chelating resin, and further may be Chelex resin.

In some embodiments, the liquefaction component for preparing the liquefying agent of the second aspect of this disclosure includes guaifenesin, a strong base and rigid microparticles. In some preferred embodiments, guaifenesin, strong base and rigid microparticles in the liquefying agent of the second aspect of this disclosure are provided in a mixed system containing the three (collectively packaged in one container), and preferably further include a solvent, which is further preferably an aqueous solvent, and more preferably water or buffer solution. Among them, the content of guaifenesin can be preferably 1 mmol/L-1 mol/L, the content of strong base can be preferably 5 mmol/L-500 mmol/L (which can be further preferably sodium hydroxide), and the content of rigid microparticles can be preferably 0.1 g/mL-2 g/mL (which can be further preferably zirconia beads).

In some preferred embodiments, the liquefaction component for preparing the liquefying agent of the second aspect of this disclosure includes 100 mM of guaifenesin, 10 mM of strong base (independently preferably sodium hydroxide) and 1 g/mL of rigid microparticles (independently preferably zirconia beads).

In some preferred embodiments, the liquefaction component for preparing the liquefying agent of the second aspect of this disclosure is consisted of guaifenesin, strong base, rigid microparticles and aqueous solvent.

In some embodiments, the liquefying agent of the second aspect of this disclosure includes (final concentration):100 mM of guaifenesin, 10 mM of sodium hydroxide and 1 g/mL of rigid microparticles (preferably zirconia beads) with a solvent being water.

The components in the liquefaction component for preparing the liquefying agent of the second aspect of this disclosure can be packaged separately or in combination.

In some embodiments, the liquefaction component for preparing the liquefying agent of the second aspect of this disclosure is a composition packaged in a container. In some embodiments, one packaging container contains guaifenesin and strong base in the liquefaction component, and another packaging container contains rigid microparticles. In some embodiments, guaifenesin, strong base, and rigid microparticles are individually packaged separately. In this disclosure, the composition can be a solid (preferably a dry powdery solid) or a liquid, or a state of being somewhere in between, such as a gelatinous state. In some embodiments, the composition is a solution. The solvent of the solution can be water, for example, distilled water, purified water, filtered water, deionized water, etc; preferably nucleic acid free water, more preferably nuclease free water.

In some embodiments, the liquefaction component composition for preparing the liquefying agent of the second aspect of this disclosure is a mixture (packaged together), which may include rigid microparticles. Further, the concentration of rigid microparticles may be 0.1 g/mL-2.0 g/mL, for example, 0.1 g/mL, 0.2 g/mL, 0.3 g/mL, 0.4 g/mL, 0.5 g/mL, 0.6 g/mL, 0.7 g/mL, 0.8 g/mL, 0.9 g/mL, 1.0 g/mL, 1.1 g/mL, 1.2 g/mL, 1.3 g/mL, 1.4 g/mL, 1.5 g/mL, 1.6 g/mL, 1.7 g/mL, 1.8 g/mL, 1.9 g/mL, 2.0 g/mL, etc. The concentration of rigid microparticles can also be selected from any one of the foregoing concentrations of rigid microparticles (including but not limited to those of the first aspect of this disclosure and the second aspect of this disclosure).

In a third aspect of this disclosure, there is provided a viscous biological sample processing combination product, which includes a liquefaction component comprising the viscous biological sample liquefying composition as described in the first aspect of this disclosure, or the liquefaction component is a combination product or a single liquefying agent, wherein, the single liquefying agent is selected from the liquefying agent of the second aspect of this disclosure.

In this disclosure, “single liquefying agent” refers to the mixture of liquefaction components that can be packaged in an independent container, may be pure liquid (such as a solution), or may be a solid-liquid mixture (such as when rigid microparticles are included).

In some embodiments, the viscous biological sample processing combination product further includes a preservation component, a nucleic acid releasing component or a combination thereof.

In some embodiments, the viscous biological sample processing combination product includes the liquefaction component and the preservation component, at this point, the viscous biological sample processing combination product can be denoted as a combination product for liquefying and preserving viscous biological sample.

In some embodiments, the viscous biological sample processing combination product includes the liquefaction component and the nucleic acid releasing component, at this point, the viscous biological sample processing combination product can be denoted as a combination product for liquefying and releasing viscous biological sample.

In some embodiments, the viscous biological sample processing combination product includes the liquefaction component, the preservation component, and the nucleic acid releasing component.

In some embodiments, the viscous biological sample processing combination product includes the single liquefying agent.

In some embodiments, the viscous biological sample processing combination product includes the preservation agent.

In some embodiments, the viscous biological sample processing combination product includes the releasing agent.

In some embodiments, the viscous biological sample processing combination product includes the single liquefying agent and the preservation agent.

In some embodiments, the viscous biological sample processing combination product includes the single liquefying agent and the releasing agent.

In some embodiments, the viscous biological sample processing combination product includes the single liquefying agent, the preservation agent and the releasing agent.

In this disclosure, “preservation agent” refers to an independent system, which is a mixture consisted of preservation components, unless otherwise limited. The different components in the “preservation component” can be independent agents or non-independent components.

The preservation component as used in this disclosure is to ensure the stable preservation of liquefied samples, which is convenient for clinical follow-up operations in a flexible time. In this disclosure, “preservation system” refers to a mixed system formed by preservation components and substances to be preserved, unless otherwise limited.

In this disclosure, the liquefaction component and the preservation component should be packaged separately in principle. The viscous biological sample is firstly processed with the liquefaction component under alkali conditions, and the viscosity is reduced by liquefaction, then the pH value of the liquefied sample is lowered and maintained at pH 6-pH 8 by using the buffer component in the preservation component. The components in the preservation component cooperate with each other, which can preserve the liquefied sample for a long time and effectively avoid degradation of nucleic acids, so as to perform the subsequent operations.

In this disclosure, “releasing agent” refers to an independent system, which is a mixture consisted of nucleic acid releasing component, unless otherwise limited. The different components in the “nucleic acid releasing component” can be independent agents or non-independent components. In this disclosure, “releasing component” and “nucleic acid releasing component” have the same meanings and can be used interchangeably, unless otherwise defined. In this disclosure, “releaser” and “nucleic acid releasing agent” have the same meanings and can be used interchangeably, unless otherwise defined.

In this disclosure, the respective components of the nucleic acid releasing component can be mixed to obtain an independent releasing agent for releasing nucleic acids in the viscous biological sample or the processed viscous biological sample.

For example, “nucleic acid releasing component” herein refers to the component used to release nucleic acid, that is, the component used to realize the release of nucleic acids. The term “the release of nucleic acids” refers to the release of nucleic acids from the sample and being at a state that can be extracted/enriched/purified/detected. The release of nucleic acids is usually accompanied by cell lysis and the physical separation of impurity components such as protein, lipid, polysaccharide, etc., from nucleic acid components by simple methods such as centrifugation, etc., which is different from the situation at physiological conditions, and nucleic acids can be directly detected by some detection reagents. “A state that can be extracted/enriched/purified/detected” refers to an extractable, enrichable, purifiedable or detectable state.

In this disclosure, “combination product” means that the components therein are not all mixed together, and one or more components therein can be individually packaged separately.

In some embodiments, a separate package includes both guaifenesin and strong base.

In some embodiments, the liquefaction component corresponds to an independent package.

In some embodiments, the preservation component corresponds to an independent package.

In some embodiments, the nucleic acid releasing component corresponds to an independent package.

In some embodiments, a combination product for liquefying and preserving viscous biological sample is provided, which includes the liquefaction component and the preservation component.

The liquefaction component in the combination product for liquefying and preserving viscous biological sample can quickly liquefy the viscous biological sample, reduce the viscosity of the sample, facilitate the subsequent operation, perform at room temperature without harsh operating conditions, has no adverse effect on subsequent nucleic acid detection, and enable effective preservation of the liquefied biological sample for a long time. The combination product for liquefying and preserving viscous biological sample provided in this disclosure also has good compatibility and has no adverse impact on the subsequent nucleic acid detection, and thus, it is compatible with the extraction free amplification system and can directly carry out the subsequent operations such as nucleic acid amplification, detection etc., without nucleic acid purification.

The respective components of the preservation component can be mixed to obtain an independent preservation agent for preserving the viscous biological sample or the processed viscous biological sample.

In some embodiments of this disclosure, the preservation component includes the following components:
a) the buffer component (denoted as the third buffer component) used to adjust the pH of the preservation system to 6-8;
b) an osmotic pressure regulating component; and
c) at least one of trehalose, mannitol or glycerol.

In some embodiments of this disclosure, the preservation component constitutes the preservation agent, which can be independently packaged in an independent container.

In this disclosure, the buffer component of component a) can be used to neutralize the strong base in the liquefaction component and/or nucleic acid releasing component, and lower the pH environment of the liquefied sample to a mild range, such as pH 6-pH 8, so as to provide a stable preservation environment for the nucleic acids in the liquefied sample.

In some embodiments of this disclosure, component a) can adjust the pH of the preservation system to 6-8.

In some embodiments of this disclosure, the third buffer component corresponds to an independent package.

In this disclosure, the definition of the buffer component in the preservation component is consistent with the buffer component in the liquefaction component and nucleic acid releasing component, both of which play a role in maintaining a specific pH. It should be understood that the definitions of buffer component in preservation component, liquefaction component and nucleic acid releasing component are independent, which are respectively denoted as the third buffer component, the first buffer component and the second buffer component. They can be the same or different from each other, and each can be independently selected from the “buffer component” as above defined in this disclosure.

In this disclosure, the buffer component in the preservation component is to neutralize the base. When the sample is processed by the liquefaction component and mixed with the preservation component, the pH of the mixed system is roughly neutral, for example, pH 6-pH 8, pH 7±0.2, etc. In some embodiments of this disclosure, the concentration of the buffer component in the preservation component may be: 1 mmol/L-5 mmol/L of citric acid, or the example specific concentration of 1 mmol/L, 2 mmol/L, 3 mmol/L, 4 mmol/L, 5 mmol/L, etc., can be selected. The concentration here, refers to the final concentration in the preservation component, unless otherwise limited. The components of the buffer components can be packaged independently in one or more containers. When its components are packaged independently in one container, it can be used as a prefabricated buffer reagent.

In this disclosure, for the buffer component in the preservation component, the buffer and buffer solution are generally prepared from buffer salt or preferably non-ionic buffer components such as TRIS and HEPES, and further can be selected from weak acid and/or its salt. The buffer component that can be used is preferably at least one selected from a) citric acid, acetic acid, phosphoric acid, tartaric acid, malic acid, carbonic acid, barbituric acid, or one or more components selected from the group consisted of b) acid radical of a, or c) acidic acid radical of a (usually carrying one or two hydrogen ions, for example, hydrogen phosphate radical, dihydrogen phosphate radical), or selected from a), b), and c). In some embodiments of this disclosure, the concentration of the buffer component in the preservation component may be: 1 mmol/L-5 mmol/L of citric acid, or the example specific concentration of 1 mmol/L, 2 mmol/L, 3 mmol/L, 4 mmol/L, 5 mmol/L, etc., can be selected. In some embodiments of this disclosure, the buffer component that can be used for i) is selected from 0.5 mmol/L-500 mmol/L of Tris-HCl, the concentration of which can also be selected from 1 mmol/L, 5 mmol/L, 10 mmol/L, 50 mmol/L, 100 mmol/L, 200 mmol/L, 300 mmol/L and 400 mmol/L.

In some embodiments of this disclosure, the preservation component meets one or more of the characteristics as shown in (tb1), (tb2), (tb3), (tb4), (tb5) and (tb6).

In some embodiments of this disclosure, the preservation component meets one or more of the characteristics as shown in (tb2a), (tb3a), (tb5a), (tb4a), (tb4b), (tb4c) and (tb6a).

In some embodiments of this disclosure, the preservation component meets the characteristic (tb1): the buffer component is used to adjust the pH of the preservation system to 6.8-7.6; further preferably 6.8-7.4; further preferably 6.8-7.2; further preferably 7.0-7.6; further preferably 7.0-7.4; further preferably 7.0-7.2; further preferably 7.2-7.4.

In some embodiments of this disclosure, the preservation component meets the characteristic (tb2): the buffer component includes citric acid.

In some embodiments of this disclosure, the preservation component meets the characteristic (tb2a): the buffer component includes 1 mmol/L-5 mmol/L of citric acid; further preferably, the buffer component includes 1.5 mmol/L-4.5 mmol/L of citric acid; further preferably, the buffer component includes 2 mmol/L-4 mmol/L of citric acid; further preferably, the buffer component includes 2.5 mmol/L-3.5 mmol/L of citric acid; further preferably, the buffer component is 1 mmol/L-5 mmol/L of citric acid.

In this disclosure, the osmotic pressure regulating component in the preservation component is used to stabilize the osmotic pressure of the biological component (for example, cells or viruses) to be preserved. Osmotic pressure regulating components usually include inorganic cations (especially salt ions, preferably Na⁺ and K⁺) and/or betaine. In one embodiment, the osmotic pressure regulating component includes 0.1%-1.2% (m/v) of sodium chloride and 0.1%-1.2% (m/v) of potassium chloride. In one embodiment, betaine 0.1%-10% (m/v). In this disclosure, the concentration of the osmotic pressure regulating component refers to the final concentration in the preservation component, unless otherwise limited.

In some embodiments of this disclosure, the preservation component is provided in a single mixed system, which can be prepared by adding additional components to the buffer component.

In some embodiments of this disclosure, the preservation component meets the characteristic (tb3): the osmotic pressure regulating component includes betaine and inorganic cations or a combination thereof; further preferably, the inorganic cation includes Na⁺, K⁺ or a combination thereof; further preferably, the inorganic cation is Na⁺, K⁺ or a combination thereof; further preferably, the osmotic pressure regulating component includes sodium chloride and potassium chloride; further preferably, the osmotic pressure regulating component is a combination of sodium chloride and potassium chloride.

In some embodiments of this disclosure, the preservation component meets the characteristic (tb3a): the osmotic pressure regulating component includes 0.1%-1.2% (m/v) of sodium chloride and 0.1%-1.2% (m/v) of potassium chloride.

In some embodiments of this disclosure, component c) in the preservation component includes 0.8 mol/L-1 mol/L of glycine and 0.6 mol/L-1 mol/L of isoleucine, based on the final concentration in the preservation component.

In some embodiments of this disclosure, the concentration of trehalose in the preservation component is 0.5 mol/L-1 mol/L, for example, 0.5 mol/L, 0.6 mol/L, 0.7 mol/L, 0.8 mol/L, 0.9 mol/L, 1 mol/L, etc.

In some embodiments of this disclosure, the concentration of mannitol in the preservation component is 1.5%-4.5% (m/v), further may be 1.5%, 2%, 2.5%, 3%, 3.5%, 4%, 4.5%, etc.

In some embodiments of this disclosure, the concentration of the glycerol in the preservation component is 2%-10% (v/v), further may be 2%, 3%, 4%, 5%, 6%, 7%, 8%, 9%, 10%, etc.

In some embodiments of this disclosure, the preservation component meets the characteristic (tb4): the preservation component includes trehalose, mannitol and glycerol.

In some embodiments of this disclosure, the preservation component meets the characteristic (tb4a): the concentration of trehalose in the preservation component is 0.5 mol/L-1 mol/L.

In some embodiments of this disclosure, the preservation component meets the characteristic (tb4b): the concentration of mannitol in the preservation component is 1.5%-4.5% (m/v).

In some embodiments of this disclosure, the preservation component meets the characteristic (tb4c): the concentration of glycerol in the preservation component is 2%-10% (v/v).

In some embodiments of this disclosure, the preservation component further includes one or more amino acids. Amino acids may be laevo- or dextro-chiral amino acids; may be naturally occurring amino acids, further may be non-naturally occurring amino acids; examples of amino acids include, but are not limited to: glycine, alanine, valine, leucine, isoleucine, methionine (methionine), proline, tryptophan, serine, tyrosine, cysteine, phenylalanine, asparagine, glutamine, threonine, aspartic acid, glutamic acid, lysine, arginine, histidine, selenocysteine and pyrrolysine, etc. In some preferred embodiments, total concentration of amino acids in the preservation components is preferably 1 mol/L-3 mol/L, for example, 1 mol/L, 1.4 mol/L, 1.5 mol/L, 1.8 mol/L, 2 mol/L, 2.2 mol/L, 2.5 mol/L, 3 mol/L, etc.

In some embodiments of this disclosure, the preservation component meets the characteristic (tb5): the preservation component further includes one or more amino acids.

In some embodiments of this disclosure, the preservation component meets the characteristic (tb5a) the preservation component further includes one or more amino acids, and the total concentration of the amino acid in the preservation component is 1 mol/L-3 mol/L; further preferably, the total concentration of the amino acid in the preservation component is 1.5 mol/L-3 mol/L or 1 mol/L-2.5 mol/L; further preferably, the total concentration of the amino acid in the preservation component is 1.5 mol/L-2.5 mol/L; further preferably, the preservation component includes glycine and isoleucine; further preferably, the preservation components include 0.8 mol/L-1 mol/L of glycine and 0.6 mol/L-1 mol/L of isoleucine; further preferably, the amino acid ingredients in the preservation component are glycine and isoleucine; further preferably, the amino acid ingredient in the preservation component is 0.8 mol/L-1 mol/L of glycine and 0.6 mol/L-1 mol/L of isoleucine.

In some embodiments of this disclosure, the preservation component further includes urea. The concentration of urea may be 1%-3% (m/v), for example, 1%, 1.5%, 2%, 2.5%, 3% (m/v), etc., based on the final concentration in the preservation component.

In some embodiments of this disclosure, the preservation component meets the characteristic (tb6): the preservation component further includes urea.

In some embodiments of this disclosure, the preservation component meets the characteristic (tb6a): the preservation component further includes 1%-3% (m/v) of urea.

In some embodiments of this disclosure, the preservation component includes (at a final concentration): 1 mmol/L-5 mmol/L of citric acid, 0.1%-1.2% (w/v) of sodium chloride, 0.1%-1.2% (w/v) of potassium chloride, 0.8 mol/L-1 mol/L of glycine, 0.6 mol/L-1 mol/L of isoleucine, 0.5 mol/L-1 mol/L of trehalose, 1.5%-4.5% (w/v) of mannitol and 2%-10% (v/v) of glycerol.

In some embodiments of this disclosure, the preservation component is provided in a single mixed system, which can be prepared by adding additional components to the buffer component. In some embodiments, the osmotic pressure regulating component is preferably 0.1%-1.2% (w/v) of sodium chloride, more preferably 1% (w/v) sodium chloride, the component c) in the preservation component is preferably 0.5 mol/L-1 mol/L of trehalose, further preferably 1 M trehalose.

In some embodiments of this disclosure, a combination product for liquefying and releasing viscous biological sample is provided, which includes the liquefaction component and the nucleic acid releasing component.

The liquefaction component in the combination product for liquefying and releasing viscous biological sample can quickly liquefy the viscous biological sample, reduce the viscosity of the sample, facilitate the subsequent operation, perform at room temperature without harsh operating conditions, and has no adverse effect on subsequent nucleic acid detection The nucleic acid releasing component provided can be used together with the liquefaction component, and has better release effect on RNA and DNA, respectively. The combination product for liquefying and releasing viscous biological sample provided in this disclosure also has good compatibility and has no adverse impact on the subsequent nucleic acid detection and thus, it is compatible with the extraction free amplification system and can directly carry out the subsequent operations such as nucleic acid amplification, detection, etc., without nucleic acid purification.

In some embodiments of this disclosure, the nucleic acid releasing component is a composition as shown in component i), a composition as shown in component ii), or a composition comprising components i) and ii).

In this disclosure, component i) can include: 0.1%-2% (v/v) of Tween 20, 0.1%-3% (v/v) of Triton X-100, 0.1%-3% (v/v) of ethyl phenyl polyethylene glycol, 50 mmol/L-1.25 mol/L of strong base (denoted as the second strong base), an adsorbing agent (denoted as the first adsorbing agent) and an aqueous solvent (denoted as the second aqueous solvent).

In this disclosure, component ii) can include: 0.01 mmol/L-0.5 mmol/L of surfactant, 0.01%-2% (w/v) of dodecyl benzene sulfonate, 0.05%-1% (v/v) of ethanol and 100 mmol/L-1.25 mol/L of strong base (denoted as the third strong base), with or without an adsorbing agent (denoted as the second adsorbing agent).

In some embodiments of this disclosure, the nucleic acid releasing component constitutes the nucleic acid releasing agent, which can be independently packaged in an independent container.

In some embodiments of this disclosure, the component i) further includes 20 mmol/L-1 mol/L of a second inorganic cation; preferably, the second inorganic cation is Na⁺, K⁺ or a combination thereof.

In some embodiments of this disclosure, the component ii) further includes 50 mmol/L-1.2 mol/L of a third inorganic cation; preferably, the third inorganic cation is Na⁺, K⁺ or a combination thereof.

In some embodiments of this disclosure, the nucleic acid releasing component is a composition as shown in component i), a composition as shown in component ii), or a composition comprising components i) and ii):
The component i) includes: 0.1%-2% (v/v) of Tween 20, 0.1%-3% (v/v) of Triton X-100, 0.1%-3% (v/v) of ethyl phenyl polyethylene glycol, 20 mmol/L-1 mol/L of a second inorganic cation (preferably Na⁺ and/or K⁺), 50 mmol/L-1.25 mol/L of a strong base (denoted as the second strong base), an adsorbing agent (denoted as the first adsorbing agent) and an aqueous solvent (denoted as the second aqueous solvent);
The component ii) includes: 0.01 mmol/L-0.5 mmol/L of surfactant, 0.01%-2% (w/v) of dodecyl benzene sulfonate, 50 mmol/L-1.2 mol/L of a third inorganic cation (preferably Na⁺ and/or K⁺), 0.05%-1% (v/v) of ethanol, and 100 mmol/L-1.25 mol/L of a strong base (denoted as the third strong base), also optionally includes an adsorbing agent (also denoted as a second adsorbing agent); and further includes a solvent (preferably water). Herein, “Na⁺ and/or K⁺” refers to “Na⁺, K⁺ or a combination thereof”.

The component ii) is released by the following ways: cell lysis and releasing of nucleic acids.

The component i) is released by the following ways: cell lysis, releasing of nucleic acids, and maintaining the structural stability of nucleic acids (RNA is easy to degrade).

When component i) is used in admixture with component ii), the cells are lysed to release nucleic acids of which the structural is kept stable.

In this disclosure, the first strong base in the liquefaction component, the second strong base in the nucleic acid releasing component and the third strong base in the nucleic acid releasing component are independent of each other, and they can be the same or different from each other.

In some embodiments of this disclosure, the concentration of Tween 20 in component i) may be 0.1%-2% (v/v), further may be 0.5%-1.5% (v/v), more further may be 0.8%-1.2% (v/v), for example, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, etc., in percentage of volume.

In some embodiments of this disclosure, the concentration of Triton X-100 in component i) may be 0.1%-3% (v/v), further may be 0.2%-2% (v/v), still further may be 0.5%-1.5% (v/v), yet still further may be 0.8%-1.2% (v/v), for example, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.2%, 2.4%, 2.5%, 2.6%, 2.8%, 3%, etc., in percentage of volume.

In some embodiments of this disclosure, ethyl phenyl polyethylene glycol in component i) is in liquid state, and the molecular weight of the polyethylene glycol moiety is not particularly limited, provided that it renders a liquid state or can be completely dissolved in component i). The concentration of ethyl phenyl polyethylene glycol in component i) may be 0.1%-3% (v/v), further may be 0.2%-2% (v/v), still further may be 0.5%-1.5% (v/v), yet still further may be 0.8%-1.2% (v/v), for example, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.2%, 2.4%, 2.5%, 2.6%, 2.8%, 3%, etc., in percentage of volume.

In some embodiments of this disclosure, the concentration of Na⁺ and K⁺ in component i) may be each independently 20 mmol/L-1 mol/L, further may be 60 mmol/L-500 mmol/L, still further may be 60 mmol/L-200 mmol/L, yet still further may be 60 mmol/L-100 mmol/L, each independently for example, 60 mmol/L, 70 mmol/L, 80 mmol/L, 90 mmol/L, 100 mmol/L, 150 mmol/L, 200 mmol/L, 250 mmol/L, 500 mmol/L, etc. In one embodiment, the concentration of Na is 100 mM and the concentration of K⁺ is 80 mM. Na⁺, and K⁺ each independently can be provided by the corresponding salt and/or base, for example, NaCl, KCl, NaOH, KOH, etc. In one embodiment, component i) contains 100 mM of NaCl, 80 mM of KCl and 250 mm of NaOH. In some embodiments, Na⁺ and K⁺ (i.e., sodium salt and potassium salt, for example, sodium chloride and potassium chloride respectively) are provided in the form of salt, and the concentration of Nae and K⁺ provided in the form of salt may be each independently 60 mmol/L-500 mmol/L, further may be 60 mmol/L-500 mmol/L, still further may be 60 mmol/L-200 mmol/L, yet still further may be 60 mmol/L-100 mmol/L, each independently for example, 60 mmol/L, 70 mmol/L, 80 mmol/L, 90 mmol/L, 100 mmol/L, 150 mmol/L, 200 mmol/L, 250 mmol/L, 500 mmol/L, etc.

In some embodiments of this disclosure, in component ii), the surfactant may be one or more of Surfactin, sodium dodecyl sulfate (SDS), ethylenediamine tetraacetic acid (EDTA), etc. In some preferred embodiments, the surfactant is Surfactin. The concentration of surfactant (preferably Surfactin) in component ii) may be 0.01 mmol/L-0.5 mmol/L, further may be 0.1 mmol/L-0.5 mmol/L, still further may be 0.2 mmol/L-0.5 mmol/L, and specifically for example, 0.01 mM, 0.05 mM, 0.1 mM, 0.15 mM, 0.2 mM, 0.25 mM, 0.3 mM, 0.35 mM, 0.4 mM, 0.45 mM, 0.5 mM, etc.

In some embodiments of this disclosure, in component ii), dodecylbenzene sulfonate may be a sodium salt. The concentration of dodecylbenzene sulfonate in component ii) may be 0.01%-2% (w/v), further may be 0.1%-2% (w/v), still further may be 0.1%-1.5% (w/v) or 0.5%-2% (w/v), yet still further may be 0.1%-1.5% (w/v), yet still further may be 0.5%-1.5% (w/v), yet still further may be 0.8%-1.2% (w/v), and specifically for example, any one percentage or an interval between any two percentages in the following group: 0.01%, 0.05%, 0.1%, 0.15%, 0.2%, 0.25%, 0.3%, 0.35%, 0.4%, 0.45%, 0.5%, 0.55%, 0.6%, 0.7%, 0.8%, 0.9%, 1%, 1.1%, 1.2%, 1.3%, 1.4%, 1.5%, 1.6%, 1.7%, 1.8%, 1.9%, 2%, etc., in mass volume ratio. In some preferred examples of this disclosure, the concentration of dodecylbenzene sulfonate in component ii) may be 0.01%-1% (w/v).

In some embodiments of this disclosure, in component ii), Na⁺ and/or K⁺ are preferably provided in the form of salt (sodium salt and/or potassium salt), for example, sodium chloride, and potassium chloride. The concentration of Na and K⁺ in component ii) each independently may be 50 mM-1.2 M, further each independently may be 50 mM-1 M, still further may be 50 mM-500 mM, yet still further may be 80 mM-500 mM, specifically for example, 50 mM, 60 mM, 70 mM, 80 mM, 90 mM, 100 mM, 110 mM, 120 mM, 130 mM, 140 mM, 150 mM, 160 mM, 180 mM, 200 mM, 300 mM, 350 mM, 400 mM, 450 mM, 500 mM, 550 mM, 600 mM, 650 mM, 700 mM, 750 mM, 800 mM, 850 mM, 900 mM, 950 mM, 1 M, etc. In some embodiments, the concentration of sodium salt and/or potassium salt (e.g., sodium chloride, potassium chloride) in component ii) is 50 mM-200 mM, further may be 50 mM-150 mM, still further may be 50 mM-120 mM, yet still further may be 80 mM-120 mM, specifically, for example, 50 mM, 60 mM, 70 mM, 80 mM, 90 mM, 100 mM, 110 mM, 120 mM, 130 mM, 140 mM, 150 mM, 160 mM, 180 mM, 200 mM, etc.

In some embodiments of this disclosure, the concentration of ethanol in component ii) may be 0.05%-1% (v/v), further may be 0.1%-1% (v/v), still further may be 0.1%-0.8% (v/v), yet still further may be 0.2%-0.8% (v/v), specifically for example, 0.05%, 0.1%, 0.15%, 0.2%, 0.25%, 0.3%, 0.35%, 0.4%, 0.45%, 0.5%, 0.55%, 0.6%, 0.7%, 0.8%, 0.9%, 1%, etc, in percentage of volume.

In this disclosure, the first strong base, the second strong base and the third strong base are all strong bases. The first strong base is contained in the liquefaction component, which is used to provide an alkali environment for the liquefaction of the viscous biological sample. The second strong base and the third strong base are contained in the nucleic acid releasing component to ensure the release of nucleic acids in a strong alkali environment. In this disclosure, the first strong base, the second strong base and the third strong base are each defined independently, which can be the same or different, and are each independently selected from the “strong base” as defined in this disclosure.

In some embodiments of this disclosure, the second strong base in the component i) is sodium hydroxide and/or potassium hydroxide.

In some embodiments of this disclosure, the second strong base is a monobasic base (for example, sodium hydroxide and/or potassium hydroxide), the concentration of which is 50 mM-1.25 M. Examples of concentration ranges include, but are not limited to 50 mM-1 M, 50 mM-800 mM, 50 mM-600 mM, 50 mM-500 mM, 50 mM-400 mM, 50 mM-200 mM, 100 mM-1 M, 100 mM-800 mM, 100 mM-600 mM, 100 mM-500 mM, 100 mM-400 mM, 200 mM-800 mM, 200 mM-500 mM, 200 mM-400 mM, etc. Examples of concentrations include, but are not limited to 50 mM, 60 mM, 70 mM, 80 mM, 90 mM, 100 mM, 120 mM, 140 mM, 150 mM, 200 mM, 250 mM, 300 mM, 350 mM, 400 mM, 450 mM, 500 mM, 600 mM, 700 mM, 800 mM, 900 mM, 1 M, etc. The technical features in this embodiment can be combined in other embodiments in a suitable manner.

In some embodiments of this disclosure, the second strong base is at least one of sodium hydroxide, potassium hydroxide, lithium hydroxide, calcium hydroxide, barium hydroxide, choline, etc.

In some embodiments of this disclosure, the second strong base is sodium hydroxide and/or potassium hydroxide. Further, the concentration of the second strong base in component i) is 50 mM-1.25 M, still further may be 50 mM-1 M.

In some embodiments of this disclosure, the third strong base is a monobasic base (for example, sodium hydroxide and/or potassium hydroxide), the concentration of which is 100 mM-1.25 M. Examples of concentration ranges include, but are not limited to 100 mM-1 M, 100 mM-900 mM, 100 mM-800 mM, 100 mM-600 mM, 100 mM-500 mM, 100 mM-400 mM, 150 mM-1 M, 150 mM-800 mM, 150 mM-1600 mM, 150 mM-500 mM, 150 mM-400 mM, 200 mM-800 mM, 200 mM-500 mM, 200 mM-400 mM, etc. Examples of concentration ranges include, but are not limited to 100 mM, 120 mM, 140 mM, 150 mM, 200 mM, 250 mM, 300 mM, 350 mM, 400 mM, 450 mM, 500 mM, 600 mM, 700 mM, 800 mM, 900 mM, 1 M, etc. The technical features in this embodiment can be combined in other embodiments in a suitable manner.

In some embodiments of this disclosure, the second strong base is at least one of sodium hydroxide, potassium hydroxide, lithium hydroxide, calcium hydroxide, barium hydroxide, choline, etc.

In some embodiments of this disclosure, the third strong base is sodium hydroxide and/or potassium hydroxide. Further, the concentration of the third strong base in component ii) is 150 mM-1.25 M, further may be 150 mM-1 M.

In some embodiments of this disclosure, the nucleic acid releasing component meets the characteristic (tr4): the second strong base in the component i) is at least one of sodium hydroxide, potassium hydroxide, lithium hydroxide, calcium hydroxide, barium hydroxide or choline; further preferably, the second strong base is sodium hydroxide, potassium hydroxide or a combination thereof.

In some embodiments of this disclosure, the nucleic acid releasing component meets the characteristic (tr5): the third strong base in the component ii) is at least one of sodium hydroxide, potassium hydroxide, lithium hydroxide, calcium hydroxide, barium hydroxide or choline; further preferably, the third strong base is sodium hydroxide, potassium hydroxide or a combination thereof.

In this disclosure, the first aqueous solvent is one of the components of the liquefaction component, and the second aqueous solvent is one of the components of component i). The first aqueous solvent and the second aqueous solvent are each independently defined, each independently selected from the “aqueous solvent” as defined in this disclosure, which can be the same or different.

In some embodiments of this disclosure, the second aqueous solvent is water, for example, distilled water, purified water, filtered water, deionized water, etc; preferably nucleic acid free water, more preferably nuclease free water. In some embodiments of this disclosure, the second aqueous solvent is a buffer component, which is denoted as the second buffer component.

In this disclosure, the first buffer component and the second buffer component are each independently defined, each independently selected from the “buffer component” as defined in this disclosure, which can be the same or different.

In this disclosure, when component i) includes a buffer component, the dosage ratio of the buffer component to the second strong base should be reasonably controlled to ensure that nucleic acids can be effectively released in an alkali environment. In component i) of some embodiments, the buffer component is Tris-HCl, and the second strong base is sodium hydroxide, wherein, the molar dosage of NaOH may be 1-5 times of Tris-HCl, further may be 1.5-5 times, for example, 1.5 times, 2 times, 2.5 times, 3 times, 3.5 times, 4 times, 4.5 times, 5 times.

In some embodiments of this disclosure, component i) includes a buffer component, which is preferably selected from: Tris-HCl, potassium dihydrogen phosphate-sodium hydroxide buffer, disodium hydrogen phosphate-potassium dihydrogen phosphate buffer, disodium hydrogen phosphate-citric acid buffer, further preferably 0.5 mmol/L-500 mmol/L of Tris-HCl, and the concentration of the Tris-HCl system further may be the example specific concentration of 1 mmol/L, 10 mmol/L, 50 mmol/L, 100 mmol/L, 200 mmol/L, 300 mmol/L, 400 mmol/L, etc.

In some embodiments of this disclosure, the second aqueous solvent is Tris-HCl, further preferably 0.5 mmol/L-500 mmol/L of Tris-HCl.

In some embodiments of this disclosure, the nucleic acid releasing component meets the characteristic (tr6), in the component i), the second aqueous solvent is 0.5 mmol/L-500 mmol/L of Tris-HCl. In this disclosure, the component i) should further include an adsorbing agent (also denoted as the first adsorbing agent) in principle, so as to better realize the release of RNA virus sample after liquefaction.

The “adsorbing agent” as used in this disclosure can adsorb impurities through physical or chemical action to reduce the interference of impurities to subsequent detections. The adsorbing agent can be of any suitable type under the principle of not adversely affecting liquefaction. The chemical actions used to adsorb impurities include but are not limited to chelation. The adsorbing agent may be resin or chelate, or cheating resin. In some embodiments, the adsorbing agent is resin. In some preferred embodiments, the adsorbing agent is resins such as polypropylethylenes, polyacrylic acids, polyvinyl alcohols, chitosans, etc, further preferably chelating resins. In some preferred embodiments, the adsorbing agent is Chelex resin.

According to the different systems, the “adsorbing agent” in this disclosure can be distinguished as “first adsorbing agent”, “second adsorbing agent”, “third adsorbing agent”, etc., corresponding to component i), component ii) and liquefaction component, etc. respectively. The three are each defined independently and each independently selected from the “adsorbing agent” as defined in this disclosure. The “first adsorbing agent”, “second adsorbing agent” and “third adsorbing agent” can be the same or different from each other, and each independently preferably chelating resin, further each independently preferably Chelex resin. The adsorbing agent is optional in liquefaction component and component ii), and is necessary for releasing RNA when it is in component i). In some embodiments, the combination product for liquefying and releasing viscous biological sample includes only the second adsorbing agent, but not the first adsorbing agent and the third adsorbing agent. In some embodiments, the combination product for liquefying and releasing viscous biological sample includes only the second adsorbing agent and the third adsorbing agent. In some embodiments, the combination product for liquefying and releasing viscous biological sample includes only the first adsorbing agent and the third adsorbing agent.

In some embodiments of this disclosure, the nucleic acid releasing component meets the characteristic (tr1): the first adsorbing agent includes chelating resin, trehalose or a combination thereof; further preferably, the first adsorbing agent includes Chelex resin, trehalose or a combination thereof; further preferably, the first adsorbing agent is chelating resin; further preferably, the first adsorbing agent is Chelex resin; further preferably, the concentration of the Chelex resin in the component i) is 1%-15% (w/v).

In some embodiments of this disclosure, the nucleic acid releasing component meets the characteristic (tr2): the first adsorbing agent includes trehalose; preferably, the concentration of trehalose in the component i) is 0.5 mol/L-1 mol/L, further preferably 0.7 mol/L-1 mol/L.

In some embodiments of this disclosure, the nucleic acid releasing component meets the characteristic (tr3): the second adsorbing agent is Chelex resin; further preferably, the concentration of the Chelex resin in the component ii) is 1%-15% (w/v). In some embodiments of this disclosure, the concentration of the first adsorbing agent is 1%-15% (w/v), for example, 0.5%, 1%, 1.5%, 2%, 3%, 4%, 5%, 6%, 7%, 8%, 9%, 10%, 11%, 12%, 13%, 14%, 15%, etc., which is the final concentration in the nucleic acid releasing component. In some preferred embodiments, the first adsorbing agent is chelating resin, further is Chelex resin.

In some embodiments of this disclosure, the concentration of the second adsorbing agent is 0.1%-15% (w/v), for example, 0.5%, 1%, 1.5%, 2%, 3%, 4%, 5%, 6%, 7%, 8%, 9%, 10%, 11%, 12%, 13%, 14%, 15%, etc., which is the final concentration in the nucleic acid releasing component. In some preferred embodiments, the second adsorbing agent is chelating resin, further is Chelex resin.

In some embodiments of this disclosure, the concentration of the third adsorbing agent is 0.1%-15% (w/v), for example, 0.5%, 1%, 1.5%, 2%, 3%, 4%, 5%, 6%, 7%, 8%, 9%, 10%, 11%, 12%, 13%, 14%, 15%, etc., which is the final concentration in the liquefaction component. In some preferred embodiments, the third adsorbing agent is chelating resin, further is Chelex resin.

In this disclosure, the addition of adsorbing agent (preferably chelating resin) to the nucleic acid releasing component can improve the sensitivity of DNA detection.

In this disclosure, the addition of adsorbing agent (preferably chelating resin) to the nucleic acid releasing component further plays a key role in the releasing of RNA. During RNA detection, reverse transcription is usually carried out first, however the reverse transcription process is easy to be interfered by impurities such as protein and polysaccharide, etc., resulting in low efficiency of RNA reverse transcription and subsequent unenforceable amplification and detection. By adding adsorbing agent, impurities can be effectively adsorbed, and the interference of impurities can be reduced or avoided, so that the subsequent amplification and detection can be carried out successfully.

In some embodiments of this disclosure, the component i) optionally includes trehalose. In some embodiments, the concentration of trehalose in the component i) is 0.5 mol/L-1 mol/L, for example, 0.5 mol/L, 0.6 mol/L, 0.7 mol/L, 0.8 mol/L, 0.9 mol/L, 1 mol/L, etc. In some embodiments, the first adsorbing agent includes 0.5 mol/L-1 mol/L of trehalose. The first adsorbing agent includes trehalose, and the concentration of trehalose in component i) is 0.5 mol/L-1 mol/L, further may be 0.7 mol/L-1 mol/L.

In some embodiments of this disclosure, the nucleic acid releasing component meets the characteristic (tr7): in the component ii), the surfactant is one or more of Surfactin, sodium dodecyl sulfate and ethylenediamine tetraacetic acid.

In some embodiments of this disclosure, the nucleic acid releasing component meets one or more of the above characteristics (tr1), (tr2), (tr3), (tr4), (tr5), (tr6) and (tr7).

In some embodiments of this disclosure, the component ii) includes: 0.01 mmol/L-0.5 mmol/L of the surfactant, 0.01%-1% (w/v) of dodecylbenzene sulfonate, 60 mmol/L-1 mol/L of the third inorganic cation, 0.05%-1% (v/v) of ethanol, 150 mmol/L-1.25 mol/L of the third strong base and the second adsorbing agent. Preferably, the third inorganic cation is Na⁺, K⁺ or a combination thereof.

In some embodiments of this disclosure, component i) includes: 0.5%-1.5% (v/v) of Tween 20, 1%-2% (v/v) of Triton X-100, 1%-2% (v/v) of ethyl phenyl polyethylene glycol, 100 mmol/L-300 mol/L of Na⁺ and/or K⁺, 150 mmol/L-350 mol/L of the second strong base (for example, sodium hydroxide and/or potassium hydroxide), 1%-10% (w/v) of an adsorbing agent (for example, Chelex resin, etc.), optional trehalose and an aqueous solvent (for example, one or more of water, buffer components, etc, preferably 50 mM-200 mM of Tris-HCl).

In some embodiments of this disclosure, component i) includes: 0.5%-1.5% (v/v) of Tween 20, 1%-2% (v/v) of Triton X-100, 1%-2% (v/v) of ethyl phenyl polyethylene glycol, 100 mmol/L-300 mol/L of Nae and/or K⁺, 150 mmol/L-350 mol/L of the second strong base (for example, sodium hydroxide and/or potassium hydroxide), 1%-10% (w/v) of an adsorbing agent (for example, Chelex resin, etc.), and an aqueous solvent (for example, one or more of water, buffer components, etc, preferably 50 mM-200 mM of Tris-HCl).

In some embodiments of this disclosure, component i) includes: 0.7%-1.3% (v/v) of Tween 20, 1.2%-1.8% (v/v) of Triton X-100, 1.2%-1.8% (v/v) of ethyl phenyl polyethylene glycol, 150 mmol/L-250 mol/L of Na and/or K⁺ (for example, sodium salt and/or potassium salt, and further for example, sodium chloride and/or potassium chloride), 200 mmol/L-300 mol/L of the second strong base (for example, sodium hydroxide or potassium hydroxide), 1%-5% (w/v) of an adsorbing agent (for example, Chelex resin, etc.), and an aqueous solvent (for example, one or more of water, buffer components, etc, preferably 50 mM-150 mM of Tris-HCl).

In some embodiments of this disclosure, component i) includes: 1% (v/v) of Tween 20, 1.5% (v/v) of Triton X-100, 1.5% (v/v) of ethyl phenyl polyethylene glycol, 100 mM of sodium salt (independently for example, sodium chloride), 80 mM of potassium salt (independently for example, potassium chloride), 250 mM of strong base (for example, sodium hydroxide or potassium hydroxide), 1%-5% (w/v) adsorbing agent (for example, Chelex resin, etc., with a dosage for example, 1%, 2%, 3%, 4%, 5%) an water (for example, sterilized purified water). Still further, it further included 100 mM of Tris-HCl.

In some embodiments of this disclosure, component ii) includes: 0.1 mmol/L-0.4 mmol/L of surfactant (for example, Surfactin), 0.1%-1.5% (w/v) of dodecyl benzene sulfonate (for example, SDS), 50 mmol/L-150 mmol/L of Na and/or K⁺ (preferably sodium salt and/or potassium salt, further sodium chloride or potassium chloride), 0.2%-0.8% (v/v) of ethanol, 250 mM-800 mM of strong base (for example sodium hydroxide or potassium hydroxide), with a solvent being water. In some embodiments of this disclosure, component ii) further includes: 1%-10% (w/v) adsorbing agent (for example, Chelex resin etc.).

In some embodiments of this disclosure, component ii) includes: 0.1 mmol/L-0.4 mmol/L of surfactant (for example, Surfactin), 0.3%-1.2% (w/v) of dodecyl benzene sulfonate (for example, SDS), 80 mmol/L-120 mmol/L of K⁺ (preferably sodium salt and/or potassium salt, further may be sodium chloride, potassium chloride), 0.3%-0.7% (v/v) of ethanol, 250 mM-750 mM of strong base (for example, sodium hydroxide or potassium hydroxide), and 1%-5% (w/v) of adsorbing agent (for example, Chelex resin, etc.), and further the solvent being water.

In some embodiments of this disclosure, component ii) includes: 0.25 mmol/L of surfactant (for example, Surfactin), 1% (w/v) of dodecyl benzene sulfonate (for example, SDS), 100 mmol/L of K⁺ (preferably sodium salt and/or potassium salt, further may be sodium chloride, potassium chloride), 0.5% (v/v) of ethanol, and 500 mM of strong base (for example, sodium hydroxide or potassium hydroxide), etc., for example, the dosage being 1%, 2%, 3%, 4% and 5%), and the solvent being water. Further, optionally it further includes 1%-5% (w/v) adsorbing agent (for example, Chelex resin).

In the combination product for liquefying and releasing a viscous biological sample, the preservation component is optional and can be combined arbitrarily.

In some embodiments of this disclosure, the combination product for liquefying and releasing a viscous biological sample further includes any suitable preservation component mentioned above.

In a fourth aspect of this disclosure, there is provided a viscous biological sample liquefying agent, which includes the following components: a liquefaction component, Surfactin and an aqueous solvent (denoted as the fourth aqueous solvent); wherein, the liquefaction component comprises the viscous biological sample liquefying composition as described in the fourth aspect of this disclosure; and further the pH of the viscous biological sample liquefying agent is greater than or equal to 10.

The viscous biological sample liquefying agent is a special liquefying agent, which can be compatible with the amplification system. Further, the viscous biological sample liquefying agent may be DNA direct amplification type, RNA direct amplification type or DNA and RNA direct amplification type.

Due to the great viscosity of viscous biological sample such as sputum samples, etc. and many interfering components, when nucleic acid detection is carried out in traditional ways, the sample still need to be subjected to complex sample processing processes such as releasing, purifying, etc., after liquefaction, for removal of the factors that inhibit the PCR reaction, and then to be subjected to amplification and/or detection of nucleic acids. This leads to cumbersome steps, complex operations, time-consuming and high cost during the process from the sample collection to the sample detection, which is not suitable for mass processing of sputum samples clinically.

The viscous biological sample liquefying agent provided in the fourth aspect of this disclosure can be directly used for nucleic acid amplification and/or detection without release and extraction after liquefying the sample, which simplifies the operation, reduces the cost, reduces the detection time, and accelerates the acquisition of the detection results. This viscous biological sample liquefying agent is a direct amplification type liquefying agent, which can be provided separately, and further, the combination product for liquefying and preserving a viscous biological sample or liquefying kits can be provided.

In some embodiments of this aspect, the viscous biological sample liquefying agent includes the following components: 20 mmol/L-1 mol/L of guaifenesin, a strong base (denoted as the first strong base), 20 mmol/L-200 mmol/L of Surfactin and an aqueous solvent (denoted as the fourth aqueous solvent). Further, the first strong base is used to adjust the pH of the viscous biological sample liquefying agent greater than or equal to 10.

In some embodiments of this aspect, the mass ratio of the guaifenesine to the Surfactin is (1-5):1; and/or,
The concentration of the guaifenesine is 20 mmol/L-500 mmol/L; and/or,
the concentration of the Surfactin is 20 mmol/L-150 mmol/L; and/or,
the first strong base is sodium hydroxide and/or potassium hydroxide; and/or,
the pH of the viscous biological sample liquefying agent is 10-14.

In some embodiments of this aspect, the mass ratio of the guaifenesine to the Surfactin is (2-5):1; and/or, the concentration of the guaifenesine is 80 mmol/L-120 mmol/L; and/or,
the concentration of the Surfactin is 30 mmol/L-60 mmol/L; and/or,
the pH of the viscous biological sample liquefying agent is 10-12 or 12-14.

In some embodiments of this aspect, in the viscous biological sample liquefying agent, the fourth aqueous solvent is water or the fourth buffer component.

In some embodiments of this aspect, the fourth aqueous solvent is RNase free water; and/or, the fourth buffer component is selected from one or a combination of more of Tris-HCl, potassium dihydrogen phosphate-sodium hydroxide buffer, disodium hydrogen phosphate-potassium dihydrogen phosphate buffer and disodium hydrogen phosphate-citric acid buffer.

In some embodiments of this aspect, the fourth aqueous solvent is nuclease free water.

In some embodiments of this aspect, the viscous biological sample liquefying agent further includes one or both of an adsorbing agent (denoted as the fourth adsorbing agent) and rigid microparticles.

In some embodiments of this aspect, the fourth adsorbing agent is chelating resin.

In some embodiments of this aspect, the fourth adsorbing agent is Chelex resin; and/or, the dosage of the fourth adsorbing agent in the viscous biological sample liquefying agent is 1%-15% (w/v).

In some embodiments of this aspect, the rigid microparticles are one or more selected from zirconia, silicon nitride, ceramsite, hard stainless steel, hard tungsten carbide, sintered corundum and agate; and/or,
the rigid microparticles is sphere, near sphere, ellipsoid, column, rod, polyhedron or irregular shape; and/or,
the average particle size of the rigid microparticles is 0.01 mm-10 mm; and/or,
the dosage of rigid microparticles is 0.1 g/mL-2 g/mL.

In some embodiments of this aspect, the viscous biological sample liquefying agent includes the following components: 80 mmol/L-120 mmol/L of guaifenesin, 8 mmol/L-12 mmol/L of sodium hydroxide, 0.5 g/mL-1.5 g/mL of zirconia beads with an average particle size of 0.1 mm-10 mm, 5%-10% (w/v) of adsorbing agent (denoted as the fourth adsorbing agent), 30 mmol/L-60 mmol/L of Surfactin and the fourth aqueous solvent.

In some embodiments of this aspect, the rigid microparticles are one or more selected from zirconia, silicon nitride, ceramsite, hard stainless steel, hard tungsten carbide, sintered corundum and agate; and/or,
the rigid microparticles is sphere, near sphere, ellipsoid, column, rod, polyhedron or irregular shape; and/or,
the average particle size of the rigid microparticles is 0.01 mm-10 mm; and/or,
the dosage of rigid microparticles is 0.1 g/mL-2 g/mL.

In some embodiments of this aspect, the viscous biological sample liquefying agent also includes 1 mmol/L-200 mmol/L of Na⁺ and/or K⁺. The viscous biological sample liquefying agent provided by the fourth aspect of this disclosure can be compatible with the amplification system.

In some embodiments of this aspect, the nucleic acid amplification can be directly carried out with the viscous biological sample liquefying agent after liquefaction. “Nucleic acid amplification” may be DNA amplification and/or RNA amplification. The nucleic acid amplification method can be any suitable DNA and/or RNA amplification method in the field.

In some embodiments of this aspect, the viscous biological sample liquefying agent provided by the fourth aspect of this disclosure is direct amplification type, further may be DNA and/or RNA direct amplification type. In some preferred embodiments, the viscous biological sample liquefying agent is PCR direct amplification type.

The viscous biological sample liquefying agent provided by the fourth aspect of this disclosure uses guaifenesin and a large dose of Surfactin as the main components, which can quickly liquefy the viscous biological sample under strong alkali conditions, reduce the viscosity of the sample, followed by direct application in the nucleic acid amplification or/and detection, and it can be directly used for amplification without release and purification, so as to achieve the effect of nucleic acid amplification or/and detection after traditional purification. Moreover, when the viscous biological sample liquefying agent is used for liquefaction operation which can be carried out easily at room temperature without strict operating conditions.

The viscous biological sample liquefying agent provided by the fourth aspect of this disclosure can quickly and efficiently liquefy the viscous biological sample in 15 min (preferably ≤10 min, more preferably ≤5 min, or even ≤3 min), thereby greatly accelerating the detection efficiency of viscous biological sample. Taking sputum as an example, it takes 5 h-24 h to detect the nucleic acids by the traditional technology; however, in this disclosure, since the operation steps of release and purification can be omitted, it not only simplifies the steps, but also reduces the duration for detection cycle to 1.5 hours or even shorter.

The viscous biological sample liquefying agent provided by the fourth aspect of this disclosure also has good compatibility, and can be compatible with the extraction free amplification system and can directly carry out the subsequent nucleic acid amplification (that is, it can be used as direct amplification liquefying agent), detection and other operations without nucleic acid purification. The time for nucleic acid detection of viscous biological sample can be further shortened. It often takes 5 h-24 h by the traditional technology, but 1.5 hours or even shorter by this application.

The viscous biological sample liquefying agent provided by the fourth aspect of this disclosure can be flexibly combined with different nucleic acid detection platforms, including but not limited to real-time quantitative fluorescent PCR instrument or portable nucleic acid detector (e.g., iPonatic mobile molecular detection system). The term “the release of nucleic acids” is consistent with the foregoing, and refers to the release of nucleic acids from the sample and being at a state that can be extracted/enriched/purified/detected. The release of nucleic acids is usually accompanied by cell lysis and the physical separation of impurity components such as protein, lipid, polysaccharide, etc., from nucleic acid components by simple methods such as centrifugation, etc., which is different from the situation being at physiological conditions, and nucleic acids can be directly detected by some detection reagents.

In the fourth aspect of this disclosure, the viscous biological sample liquefying agent is PCR direct amplification type.

In this disclosure, “direct amplification” refers to the direct nucleic acid amplification with biological samples after liquefaction. For example, if traditional liquefying agents (such as sodium hydroxide solution) are used, the biological sample cannot be directly amplified after liquefaction, but need to be subjected to other intermediate processing such as release, extraction, etc. If nuclear acid amplification and detection are directly carried out after liquefaction in a traditional manner without intermediate processing, the detection process will be interfered by the traditional liquefaction system, and the reliability of the results will be poor.

In the fourth aspect of this disclosure, processing the viscous biological sample with the liquefying agent needs to be carried out in an aqueous environment. It should be understood that the aqueous solvent can be additionally provided during the processing operation.

In some embodiments of this disclosure, the viscous biological sample liquefying agent includes the following components: guaifenesin, strong base (i.e., the first strong base) and Surfactin, wherein the first strong base can adjust pH to be greater than or equal to 10. Further, the viscous biological sample liquefying agent in the fourth aspect of this disclosure further includes Na⁺ and/or K⁺.

The viscous biological sample liquefying agent in the fourth aspect includes: guaifenesin, strong base (i.e., the first strong base) and Surfactin, wherein the first strong base can adjust pH to be greater than or equal to 10.

The viscous biological sample liquefying agent in the fourth aspect also includes an aqueous solvent (i.e., the fourth aqueous solvent).

Optionally, the viscous biological sample liquefying agent in the fourth aspect may further include sodium ions and/or potassium ions (Na⁺ and/or K⁺).

Optionally, the viscous biological sample liquefying agent in the fourth aspect may further include one or both of the fourth adsorbing agent and rigid microparticles.

In the viscous biological sample liquefying agent, a high-efficiency liquefying agent suitable for PCR direct amplification is provided by the liquefaction of viscous biological sample under specific strong alkali conditions through the synergistic effect of specific combinations of guaifenesin with Surfactin. Further, when guaifenesin and Surfactin are combined at a specific mass ratio (including but not limited to 2:1, 3:1, 5:1, etc.), better synergistic effect is exhibited. In some embodiments of this aspect, the viscous biological sample liquefying agent also includes one, two or three of an adsorbing agent (denoted as the fourth adsorbing agent) and rigid microparticles, that is, the viscous biological sample liquefying agent includes the following components: guaifenesin, the first strong base, Surfactin, the fourth aqueous solvent, the optional fourth adsorbing agent and the optional rigid microparticles.

The viscous biological sample liquefying agent can quickly and efficiently liquefy the viscous biological sample in 15 min (preferably ≤10 min, more preferably ≤5 min, or even ≤3 min), thereby greatly accelerating the detection efficiency of viscous biological sample. Taking sputum as an example, it often takes 5 h-24 h to detect nucleic acids in sputum by the traditional technology, but 1.5 hours or even shorter by this application.

In some embodiments of this aspect, the viscous biological sample liquefying agent includes the following components: 20 mmol/L-1 mol/L of guaifenesin, the first strong base, 20 mmol/L-200 mmol/L of Surfactin and the fourth aqueous solvent; the pH of the viscous biological sample liquefying agent is greater than or equal to 10. The viscous biological sample liquefying agent in the fourth aspect includes guaifenesin. Traditional guaifenesin stimulates the gastric mucosa after oral administration, which reflexively causes increased bronchial secretion and diluted sputum. The inventor of this application accidentally found that guaifenesin can directly liquefy sputum in vitro.

In some embodiments of this aspect, the concentration of guaifenesin is 20 mmol/L-1 mol/L.

In some embodiments of this aspect, the concentration of guaifenesin is 20 mmol/L-500 mmol/L.

In some embodiments of this aspect, the concentration range of guaifenesin includes but is not limited to: for example, 20 mmol/L-400 mmol/L, 20 mmol/L-200 mmol/L, 20 mmol/L-150 mmol/L, 50 mmol/L-400 mmol/L, 50 mmol/L-200 mmol/L, 50 mmol/L-150 mmol/L, etc.

In some embodiments of this aspect, the concentration of guaifenesin includes, but is not limited to, any one of or a concentration interval between any two of the following concentrations: 20 mmol/L, 40 mmol/L, 50 mmol/L, 60 mmol/L, 80 mmol/L, 100 mmol/L, 120 mmol/L, 140 mmol/L, 150 mmol/L, 160 mmol/L, 180 mmol/L, 200 mmol/L, 220 mmol/L, 240 mmol/L, 250 mmol/L, 260 mmol/L, 280 mmol/L, 300 mmol/L, 320 mmol/L, 340 mmol/L, 350 mmol/L, 360 mmol/L, 380 mmol/L, 400 mmol/L, 420 mmol/L, 450 mmol/L, 500 mmol/L, etc.

The viscous biological sample liquefying agent in the fourth aspect includes Surfactin. Surfactin is a surfactant. Prior to this disclosure, use of Surfactin in a direct amplification liquefying agent has not been reported. After great effort in research and exploration, the present inventor found that a large dosage of Surfactin in combination with guaifenesin can be directly used for amplification and/or nucleic acid detection without release and purification while providing better liquefaction effect and release effect. The present inventor also found that after liquefaction, Surfactin at a relatively low dosage (for example, when its concentration in the liquefying agent is less than 20 mmol/L) needs to be used in combination with the releasing agent to release nucleic acids first, so as to obtain a better Ct value (a smaller Ct value) in the subsequent nucleic acid amplification experiment. If nucleic acid amplification is carried out directly after liquefaction, it will lead to poor amplification efficiency (a larger Ct value). The present inventor accidentally found in his research that the releasing agent may be omitted when Surfactin at an increased dosage is used, and better results can be obtained by directly amplifying nucleic acids.

In some embodiments of this aspect, the concentration of Surfactin is 20 mmol/L-200 mmol/L. Examples of concentration ranges include, but are not limited to 20 mmol/L-150 mmol/L, 20 mmol/L-100 mmol/L, 25 mmol/L-200 mmol/L, 25 mmol/L-150 mmol/L, 25 mmol/L-100 mmol/L, 30 mmol/L-200 mmol/L, 30 mmol/L-150 mmol/L, 30 mmol/L-100 mmol/L, 30 mmol/L-100 mmol/L, 30 mmol/L-80 mmol/L, 30 mmol/L-60 mmol/L, etc. Examples of concentrations include, but are not limited to, any one of or a concentration interval between any two of the following concentrations: 20 mmol/L, 25 mmol/L, 30 mmol/L, 40 mmol/L, 50 mmol/L, 60 mmol/L, 70 mmol/L, 80 mmol/L, 90 mmol/L, 100 mmol/L, 120 mmol/L, 140 mmol/L, 150 mmol/L, 200 mmol/L, etc. If the dosage of Surfactin is insufficient, the cleavage efficiency is relatively low. If the dosage of Surfactin is too large, the subsequent nucleic acid amplification efficiency will be affected.

In the viscous biological sample liquefying agent in the fourth aspect, the present inventor found that there was a synergistic effect between guaifenesin and Surfactin. Specifically, it is found that the cleavage efficiency decreases when Surfactin is omitted, the sputum sample cannot be liquefied when guaifenesin is omitted. Only by providing both of guaifenesin and Surfactin, the corresponding liquefying agent can be directly used for subsequent nucleic acid amplification or detection. Moreover, Surfactin is irreplaceable. It is found that the nucleic acids cannot be amplified in the subsequent steps, when Surfactin is replaced by guanidinium isothiocyanate.

In some embodiments of this aspect, the mass ratio of guaifenesin and Surfactin is (1-10):1, further may be (1-8):1, still further may be (1-5):1. Further, the mass ratio of guaifenesin:Surfactin may be (1-10):1, (2-10):1, (3-10):1, (5-10):1, (1-5):1, (2-5):1, (1-3):1, etc. Specific examples of dosage ratio of guaifenesin:Surfactin in mass ratio include but are not limited to 2:1, 3:1, 4:1, 5:1.

In the viscous biological sample liquefying agent in the fourth aspect, a strong base is used to provide a strong alkali environment. Here, the strong base is denoted as the first strong base, which is used to provide a strong alkali environment with pH greater than or equal to 10. There is no special limit on the type of the strong base, provided that it can provide a pH that allows sufficient liquefaction and has no adverse effect on the subsequent PCR.

In the fourth aspect of this disclosure, the definition of the term “strong base” is consistent with that as described above.

In some embodiments of this aspect, the first strong base is a monobasic base, which is used to provide an environment with pH greater than or equal to 10. In some embodiments, the concentration of the first strong base in the viscous biological sample liquefying agent is less than 1 mol/L, and further may be less than or equal to 500 mmol/L. In some embodiments, the concentration of the first strong base in the viscous biological sample liquefying agent is 0.1 mmol/L-500 mmol/L, further may be 1 mmol/L-500 mmol/L. In some embodiments, the concentration of the first strong base in the viscous biological sample liquefying agent is 5 mmol/L-500 mmol/L. Examples of concentration ranges include, but are not limited to 0.1 mmol/L-500 mmol/L, 1 mmol/L-500 mmol/L, 5 mmol/L-500 mmol/L, 5 mmol/L-400 mmol/L, 5 mmol/L-200 mmol/L, 5 mmol/L-100 mmol/L, 5 mmol/L-80 mmol/L, 5 mmol/L-60 mmol/L, 5 mmol/L-50 mmol/L, 5 mmol/L-40 mmol/L, 10 mmol/L-200 mmol/L, 10 mmol/L-100 mmol/L, 10 mmol/L-50 mmol/L, 10 mmol/L-40 mmol/L, etc. Examples of concentrations include, but are not limited to, any one of or a concentration interval between any two of the following concentrations: 0.1 mmol/L, 0.5 mmol/L, 0.6 mmol/L, 0.8 mmol/L, 1 mmol/L, 2 mmol/L, 3 mmol/L, 4 mmol/L, 4.5 mmol/L, 5 mmol/L, 6 mmol/L, 7 mmol/L, 8 mmol/L, 9 mmol/L, 10 mmol/L, 12 mmol/L, 15 mmol/L, 20 mmol/L, 25 mmol/L, 30 mmol/L, 40 mmol/L, 50 mmol/L, 60 mmol/L, 70 mmol/L, 80 mmol/L, 90 mmol/L, 100 mmol/L, 120 mmol/L, 140 mmol/L, 150 mmol/L, 200 mmol/L, 250 mmol/L, 300 mmol/L, 350 mmol/L, 400 mmol/L, 450 mmol/L, 500 mmol/L, etc. The technical features in this embodiment can be combined in other embodiments in a suitable manner.

In some embodiments of this aspect, the first strong base is sodium hydroxide and/or potassium hydroxide. In some embodiments of this aspect, the first strong base is sodium hydroxide. Further, the concentration of the first strong base is less than 1 mol/L, may be 0.1 mmol/L-500 mmol/L, further may be 1 mmol/L-500 mmol/L, still further may be 5 mmol/L-500 mmol/L. Examples of concentration range and specific concentration of the first strong base can refer to the examples of monobasic base above, and they can be combined in a suitable manner. In some embodiments of this aspect, the pH of the viscous biological sample liquefying agent is 10-14, and the pH of the viscous biological sample liquefying agent is for example, 10, 11, 12, 13, 14, etc. In some embodiments of this disclosure, the pH of the viscous biological sample liquefying agent is 10-12. In some embodiments of this aspect, the pH of the viscous biological sample liquefying agent is 12-14.

The viscous biological sample liquefying agent includes an aqueous solvent, which is denoted as the fourth aqueous solvent.

The term “aqueous solvent” as used in this aspect is consistent with that as previously defined. The fourth aqueous solvent is a solvent or solution containing water, and may be a single solvent consisted of pure water or a mixed solvent formed by the miscibility of water with other solvents, and can further contain solute components. Solvents that can be miscible with water include but are not limited to alcohol solvents (such as methanol, ethanol, propanol, isopropanol, polyethylene glycol, etc.). The aqueous solvent is also allowed to contain salt components. In some embodiments of this aspect, the fourth aqueous solvent is water or a fourth buffer component.

In some embodiments of this aspect, the fourth aqueous solvent is water, for example, distilled water, purified water, filtered water, deionized water, etc; preferably RNA free water, and/or preferably RNase free water; preferably nucleic acid free water, and/or preferably nuclease free water.

In some embodiments, the fourth aqueous solvent is RNase free water.

In some embodiments, the fourth aqueous solvent is nuclease (including RNases and DNases) free water.

The viscous biological sample liquefying agent provided by the fourth aspect of this disclosure can achieve the corresponding technical effect without adding additional nuclease inhibitors (including RNase inhibitors and/or DNase inhibitors). The viscous biological sample liquefying agent can be used without adding additional RNase inhibitors. However, it should be understood that the technical solution of adding RNase inhibitors and/or DNase inhibitors to the viscous biological sample liquefying agent in this aspect is also within the protection scope of this disclosure. The above RNase inhibitors and DNase inhibitors are known to those skilled in the art. As an example, RNase inhibitor may be, but not limited to diethyl pyrocarbonate. As an example, DNase inhibitor may be, but not limited to ethylenediamine tetraacetic acid.

In some embodiments of this aspect, the fourth aqueous solvent is a buffer component, which is denoted as the fourth buffer component.

The definition of “buffer component” as used in the fourth aspect of this disclosure is consistent with the foregoing. Further, in this aspect, the term “buffer component” is also referred to as “buffer solution”, which refers to an aqueous solution containing a buffer component. When an acid or base is added to the aqueous solution, the aqueous solution resists the change of pH. This resistance to pH changes is due to the buffering properties of such liquids. Therefore, a solution showing buffering activity is called as a buffer or buffer solution. Buffer generally does not have unlimited ability to maintain the pH of a solution or composition. On the contrary, with a buffer, a pH may be generally kept within a specific range, for example, pH 6-pH 8, or pH greater than or equal to 10, for example. Generally, with the buffer, a pH may be kept within logarithm of its pKa±1 (see, for example, Mohan, Buffers, A guide for the preparation and use of buffers in biological systems, CALBIOCHEM, 1999). The buffer and buffer solution are generally prepared from buffer salt or preferably non-ionic buffer components such as TRIS and HEPES, and can be further selected from weak acid and/or its salt. The buffer component in i) that can be used in the method in this aspect is preferably selected from Tris-HCl having a concentration of 0.5 mmol/L-500 mmol/L or selected from 1 mmol/L, 5 mmol/L, 10 mmol/L, 50 mmol/L, 100 mmol/L, 200 mmol/L, 300 mmol/L and 400 mmol/L. The buffer component of this aspect is preferably selected from one or more of a) citric acid, acetic acid, phosphoric acid, tartaric acid, malic acid, carbonic acid, barbituric acid, or b) acid radical of a, or c) acidic acid radical of a (usually carrying one or two hydrogen ions, for example, hydrogen phosphate radical, dihydrogen phosphate radical), or one or more components selected from the group consisted of a), b), and c). The buffer component is 1 mmol/L-5 mmol/L of citric acid, or the example specific concentration of 2 mmol/L, 3 mmol/L, 4 mmol/L and the like can be selected.

In some preferred embodiments of this aspect, the fourth buffer component is one or more selected from Tris-HCl, potassium dihydrogen phosphate-sodium hydroxide buffer, disodium hydrogen phosphate-potassium dihydrogen phosphate buffer and disodium hydrogen phosphate-citric acid buffer.

In some preferred embodiments of this aspect, the buffer component is selected from any of the following buffers: Tris-HCl, potassium dihydrogen phosphate-sodium hydroxide buffer, disodium hydrogen phosphate-potassium dihydrogen phosphate buffer and disodium hydrogen phosphate-citric acid buffer, etc. In some more preferred embodiments, the buffer component is 0.5 mmol/L-500 mmol/L of Tris-HCl, the concentration of the Tris-HCl system further may be the example specific concentration of 1 mmol/L, 10 mmol/L, 50 mmol/L, 100 mmol/L, 200 mmol/L, 300 mmol/L, 400 mmol/L, etc. The concentration here refers to the final concentration in the viscous biological sample liquefying agent, unless otherwise limited. The components of the buffer components can be packaged independently in one or more containers. When its components are packaged independently in one container, it can be used as a prefabricated buffer reagent.

Further, the viscous biological sample liquefying agent optionally includes sodium ions and/or potassium ions (Na⁺ and/or K). Na⁺ and/or K⁺ is preferably provided in the form of salt (sodium salt and/or potassium salt), or can be provided together with strong bases (for example, sodium hydroxide, potassium hydroxide). Examples for sodium source and/or potassium source are sodium chloride, potassium chloride. The concentration of Na⁺ and K⁺ in the viscous biological sample liquefying agent of the fourth aspect each independently may be 1 mM-1 M, further each independently 5 mM-1 M, still further 5 mM-500 mM, yet still further 5 mM-200 mM, specifically for example, 5 mM, 10 mM, 15 mM, 20 mM, 25 mM, 30 mM, 40 mM, 50 mM, 60 mM, 70 mM, 80 mM, 90 mM, 100 mM, 110 mM, 120 mM, 130 mM, 140 mM, 150 mM, 160 mM, 180 mM, 200 mM, 300 mM, 350 mM, 400 mM, 450 mM, 500 mM, 550 mM, 600 mM, 650 mM, 700 mM, 750 mM, 800 mM, 850 mM, 900 mM, 950 mM, 1 M, etc. In some embodiments, the concentration of sodium salt and/or potassium salt (such as sodium chloride, potassium chloride) is 5 mM-250 mM, further 5 mM-150 mM, still further 5 mM-120 mM, specifically, for example, 5 mM, 10 mM, 15 mM, 20 mM, 25 mM, 30 mM, 40 mM, 50 mM, 60 mM, 70 mM, 80 mM, 90 mM, 100 mM, 110 mM, 120 mM, 130 mM, 140 mM, 150 mM, 160 mM, 180 mM, 200 mM, 210 mM, 220 mM, 230 mM, 240 mM, 250 mM, etc.

In some preferred embodiments of this aspect, the viscous biological sample liquefying agent includes the following components: 20 mmol/L-1 mol/L of guaifenesin, the first strong base, 1 mmol/L-200 mmol/L of Na⁺ and/or K⁺, 20 mmol/L-200 mmol/L of Surfactin and the fourth aqueous solvent; the pH of the viscous biological sample liquefying agent is greater than or equal to 10.

The viscous biological sample liquefying agent in the fourth aspect may also include one or both of an adsorbing agent (denoted as the fourth adsorbing agent) and rigid microparticles. That is, the fourth adsorbing agent and the rigid microparticles may be each independently used as optional components.

In some embodiments, the viscous biological sample liquefying agent in the fourth aspect includes an adsorbing agent (denoted as the fourth adsorbing agent).

The definition of “adsorbing agent” as used in the fourth aspect of this disclosure is consistent with the foregoing. Further, the “adsorbing agent” can adsorb impurities through physical or chemical action to reduce the interference of impurities to subsequent detections. The adsorbing agent can be of any suitable type under the principle of not adversely affecting liquefaction. The chemical actions for adsorbing impurities include but are not limited to chelation. The adsorbing agent may be resin or chelate, or cheating resin. In some embodiments, the adsorbing agent is resin. In some preferred embodiments, the adsorbing agent is resins such as polypropylethylenes, polyacrylic acids, polyvinyl alcohols, chitosans, etc, further preferably chelating resins. In some preferred embodiments, the adsorbing agent is Chelex resin.

In some preferred embodiments of this aspect, the fourth adsorbing agent is Chelex resin. In some embodiments, the dosage of the fourth adsorbing agent is 1%-15% (w/v), for example, 0.5%, 1%, 1.5%, 2%, 3%, 4%, 5%, 6%, 7%, 8%, 9%, 10%, 11%, 12%, 13%, 14%, 15%, etc. In some preferred embodiments, the fourth adsorbing agent is chelating resin and further may be Chelex resin.

In some embodiments of this aspect, the viscous biological sample liquefying agent of the fourth aspect optionally includes rigid microparticles. Rigid microparticles can be used to assist in mixing the viscous biological sample and liquefying agents well, reducing processing time and accelerating processing efficiency.

The definition of “rigid microparticles” as used in the fourth aspect of this disclosure is consistent with the foregoing.

In some preferred embodiments of this aspect, the rigid microparticles are made of a material including one or more of zirconia, silicon nitride, ceramsite, hard stainless steel, hard tungsten carbide, sintered corundum and agate, etc.

The definition of the term “microparticles” as used in the fourth aspect of this disclosure is consistent with the foregoing.

In some embodiments of this aspect, the “microparticle” can be a sphere, a near sphere, an ellipsoid, a column, a rod, a polyhedron (such as a cube for example) or an irregularly shape, preferably microsphere. The average particle size of microparticles is preferably in millimeter-sized, for example, 0.01 mm-500 mm, and further may be 0.05 mm, 0.1 mm, 0.2 mm, 0.3 mm, 0.4 mm, 0.5 mm, 1 mm, 2 mm, 3 mm, 4 mm, 5 mm, 6 mm, 7 mm, 8 mm, 9 mm, 10 mm, 20 mm, 30 mm, 40 mm, 50 mm, 100 mm, 200 mm, 300 mm, 400 mm and 500 mm, further preferably 0.01 mm-10 mm.

In some embodiments of this aspect, the average particle size of the rigid microparticles is 0.01 mm-10 mm; further may be 0.05 mm-10 mm; further may be 0.1 mm-10 mm; further may be 0.2 mm-10 mm; further may be 0.5 mm-10 mm; further may be 1 mm-10 mm; further may be 0.01 mm-5 mm; further may be 0.05 mm-5 mm; further may be 0.1 mm-5 mm; further may be 0.2 mm-5 mm; further may be 0.5 mm-5 mm; or further may be 1 mm-5 mm.

In some embodiments of this aspect, the rigid microparticles are spheres, cubes, polyhedrons, or irregular shapes. In some embodiments of this aspect, the dosage of rigid microparticles is 0.1 g/mL-2 g/mL. Examples of dosage include but are not limited to 0.1 g/mL, 0.2 g/mL, 0.3 g/mL, 0.4 g/mL, 0.5 g/mL, 0.6 g/mL, 0.7 g/mL, 0.8 g/mL, 0.9 g/mL, 1.0 g/mL, 1.1 g/mL, 1.2 g/mL, 1.3 g/mL, 1.4 g/mL, 1.5 g/mL, 1.6 g/mL, 1.7 g/mL, 1.8 g/mL, 1.9 g/mL, 2.0 g/mL, etc. The “dosage of rigid microparticles” here refers to the dosage of the viscous biological sample liquefying agent in the fourth aspect.

In some embodiments of this aspect, the dosage of the rigid microparticles is 0.1 g/mL-2 g/mL; further may be 0.2 g/mL-2 g/mL; further may be 0.5 g/mL-2 g/mL; further may be 0.8 g/mL-2 g/mL; further may be 1 g/mL-2 g/mL; further may be 0.1 g/mL-1.5 g/mL; further may be 0.2 g/mL-1.5 g/mL; further may be 0.5 g/mL-1.5 g/mL; further may be 0.8 g/mL-1.5 g/mL; further may be 1 g/mL-1.5 g/mL; further may be 0.1 g/mL-1.2 g/mL; further may be 0.2 g/mL-1.2 g/mL; further may be 0.5 g/mL-1.2 g/mL; further may be 0.8 g/mL-1.2 g/mL; further may be 1 g/mL-1.2 g/mL; further may be 0.1 g/mL-1 g/mL; further may be 0.2 g/mL-1.5 g/mL; further may be 0.5 g/mL-1 g/mL; or further may be 0.8 g/mL-1 g/mL.

In some embodiments of this aspect, the viscous biological sample liquefying agent in the fourth aspect includes the following components: 80 mmol/L-120 mmol/L of guaifenesin (independently preferably 100 mmol/L), 8 mmol/L-12 mmol/L of sodium hydroxide (independently preferably 10 mmol/L), 0.5 g/mL-1.5 g/mL (independently preferably 1 g/mL) of zirconia beads with an average particle size of 0.1 mm-10 mm (independently preferably 1 mm), a chelating resin with a volume percentage of 5%-10% (independently preferably 8%) and 30 mmol/L-60 mmol/L (independently preferably 50 mmol/L) of Surfactin.

In some embodiments of this aspect, the viscous biological sample liquefying agent in the fourth aspect is a mixed system made by adding the following components to the first aqueous solvent: 80 mmol/L-120 mmol/L of guaifenesin (independently preferably 100 mmol/L), 8 mmol/L-12 mmol/L of sodium hydroxide (independently preferably 10 mmol/L), 0.5 g/mL-1.5 g/mL (independently preferably 1 g/mL) of zirconia beads with an average particle size of 0.1 mm-10 mm (independently preferably 1 mm), a chelating resin with a volume percentage of 5%-10% (independently preferably 8%) and 30 mmol/L-60 mmol/L (independently preferably 50 mmol/L) of Surfactin.

In some embodiments of this aspect, the viscous biological sample liquefying agent satisfies one or more of the characteristics (tz1), (tz2), (tz3), (tz4), (tz5), (tz6) and (tz7).

In some embodiments of this aspect, the viscous biological sample liquefying agent satisfies the characteristic (tz1): the mass ratio of guaifenesin to Surfactin is (1-5):1; preferably (2-5):1; further preferably (1-3):1; further preferably (1.5-2.5):1; further preferably 1:1, 1.5:1, 2:1, 2.5:1, 3:1, 3.5:1, 4:1, 4.5:1 or 5:1.

In some embodiments of this aspect, the viscous biological sample liquefying agent satisfies the characteristic (tz2): the concentration of the guaifenesin is 20 mmol/L-500 mmol/L; preferably 80 mmol/L-120 mmol/L; further preferably 20 mmol/L-400 mmol/L; further preferably 20 mmol/L-200 mmol/L; further preferably 20 mmol/L-150 mmol/L; further preferably 50 mmol/L-400 mmol/L; further preferably 50 mmol/L-200 mmol/L; further preferably 50 mmol/L-150 mmol/L.

In some embodiments of this aspect, the viscous biological sample liquefying agent satisfies the characteristic (tz3): the concentration of Surfactin is 20 mmol/L-150 mmol/L, and preferably, the concentration of Surfactin is 30 mmol/L-60 mmol/L.

In some embodiments of this aspect, the viscous biological sample liquefying agent satisfies the characteristic (tz4): the first strong base is sodium hydroxide, potassium hydroxide or a combination thereof.

In some embodiments of this aspect, the viscous biological sample liquefying agent satisfies the characteristic (tz5): the pH of the viscous biological sample liquefying agent is greater than or equal to 10, preferably 10-14, further preferably 10-12 or 12-14, further preferably 10, 10.5, 11, 11.5, 12, 12.5, 13, 13.5 or 14.

In some embodiments of this aspect, the viscous biological sample liquefying agent satisfies the characteristic (tz6): in the viscous biological sample liquefying agent, the fourth aqueous solvent is water or a fourth buffer component; preferably, the fourth aqueous solvent is RNase free water; further preferably, the fourth aqueous solvent is nuclease free water; further preferably, the fourth buffer component is one or more selected from Tris-HCl, potassium dihydrogen phosphate-sodium hydroxide buffer, disodium hydrogen phosphate-potassium dihydrogen phosphate buffer and disodium hydrogen phosphate-citric acid buffer; and

In some embodiments of this aspect, the viscous biological sample liquefying agent satisfies the characteristic (tz7): the viscous biological sample liquefying agent further optionally contains one or both of Na⁺ and K; preferably, the concentration of Na⁺ and K⁺ in the viscous biological sample liquefying agent is each independently 1 mmol/L-1 mol/L, each independently preferably 5 mmol/L-1 mol/L, each independently and further preferably 5 mmol/L-500 mmol/L, each independently and further preferably 5 mmol/L-200 mmol/L; further preferably, the total concentration of Na⁺ and K⁺ is 1 mmol/L-250 mmol/L, preferably 1 mmol/L-200 mmol/L, further preferably 5 mmol/L-250 mmol/L, further preferably 5 mmol/L-200 mmol/L, further preferably 5 mmol/L-150 mmol/L, further preferably 5 mmol/L-120 mmol/L.

In a fifth aspect of this disclosure, there is provided a combination product for liquefying and preserving a viscous biological sample, which includes a liquefaction component (which can be used to prepare the viscous biological sample liquefying agent in the fourth aspect of this disclosure) and a preservation component (which can be used to prepare the preservation agent, and the “preservation agent” is as defined in the third aspect of this disclosure); wherein, the liquefaction component includes each component in the viscous biological sample liquefying agent as described in the fourth aspect of this disclosure.

Further, the preservation component can be as defined in the viscous biological sample processing combination product as described in the third aspect of this disclosure.

Nucleic acids (especially RNAs) in biological samples such as sputum samples, etc., are extremely unstable and are usually degraded in a few hours at room temperature. However, in clinical testing, nucleic acids in sputum samples are often not processed and detected in time. When using the combination product for liquefying and preserving, the prepared viscous biological sample liquefying agent can be used to liquefy the viscous biological sample first, and then the preservation component or the preservation agent prepared therefrom can be added, so that the liquefied sample can be stably preserved, which is convenient for the subsequent operations without release and extraction in a flexible time. The preserved samples after liquefaction can be directly amplified or detected, which is conducive to processing a large batch of samples.

In this disclosure, the liquefaction components and preservation components should be packaged separately in principle. In some embodiments, the viscous biological sample is firstly processed with the liquefaction component (or the liquefying agent obtained from the liquefaction component) under alkali conditions, and the viscosity is reduced by liquefaction, then the pH value of the liquefied sample is lowered and maintained at pH 6-pH 8 by using the buffer component in the preservation component. The components in the preservation component cooperate with each other, which can preserve the liquefied sample for a long time and effectively avoid degradation of nucleic acids, and allow the subsequent amplification or detection without release and extraction.

The liquefaction component used in the fifth aspect can refer to the components of the viscous biological sample liquefying agent in the fourth aspect of this disclosure.

In some embodiments of this aspect, the liquefaction component includes guaifenesin, the first strong base, Surfactin, Na⁺ and/or K⁺, the fourth aqueous solvent, the optional adsorbing agent and the optional rigid microparticles. The definition, type, content, optimization, examples, etc. for each component include but are not limited to those as described above, which will not be repeated here. The fourth aqueous solvent can be provided in advance or prepared by the user himself, therefore the preparation method for which is optional. However, when the viscous biological sample is processed, the fourth aqueous solvent is necessary to provide an aqueous environment.

In some embodiments of this aspect, the definition of the preservation component can refer to the third aspect of this disclosure.

In some embodiments of this aspect, the preservation component includes component a), component b) and component c), wherein,

component a) is the buffer component (denoted as the third buffer component) used to adjust the pH of the preservation system to 6-8;

component b) is an osmotic pressure regulating component; and

component c) is one, two or three of trehalose, mannitol and glycerol.

In some embodiments of this aspect, the buffer component of component a) can be used to neutralize the strong base (the first strong base) in the liquefaction component, and lower the pH environment of the liquefied sample to a mild range, such as pH 6-pH 8 (further for example, pH 6, 6.5, 7, 72, 7.4, 7.5, 8, etc.), so as to provide a stable preservation environment for the nucleic acids in the liquefied sample. The buffer components here are consistent with the definitions in the previous aspects.

In this disclosure, the first buffer component, the second buffer component, the third buffer component and the fourth buffer component are independent of each other and can be the same or different from each other.

In the fifth aspect of this disclosure, the osmotic pressure regulating component is used to stabilize the osmotic pressure of the biological component (for example, cells or viruses) to be preserved. Osmotic pressure regulating components usually include inorganic cations (especially salt ions, preferably Na⁺ and K⁺) and/or betaine. In one embodiment, the osmotic pressure regulating component includes 0.1%-1.2% (w/v) of sodium chloride and 0.1%-1.2% (w/v) of potassium chloride. In a specific embodiment, 0.1%-10% (w/v) of betaine.

In some embodiments of this aspect, the preservation component is provided in a single mixed system, which can be prepared by adding additional components to the buffer component.

In some embodiments of this aspect, the preservation component further includes one or more amino acids. Amino acids may be laevo- or dextro-chiral amino acids; may be naturally occurring amino acids, further may be non-naturally occurring amino acids; examples of amino acids include, but are not limited to: for example, glycine, alanine, valine, leucine, isoleucine, methionine (methionine), proline, tryptophan, serine, tyrosine, cysteine, phenylalanine, asparagine, glutamine, threonine, aspartic acid, glutamic acid, lysine, arginine, histidine, selenocysteine, pyrrolysine, etc. In some preferred embodiments, total concentration of amino acids in the preservation components is preferably 1 mol/L-3 mol/L, for example, 1 mol/L, 1.4 mol/L, 1.5 mol/L, 1.8 mol/L, 2 mol/L, 2.2 mol/L, 2.5 mol/L, 3 mol/L, etc.

In some embodiments of this aspect, component c) in the preservation component includes 0.8 mol/L-1 mol/L of glycine and 0.6 mol/L-1 mol/L of isoleucine.

In some embodiments of this aspect, the concentration of trehalose in the preservation component is 0.5 mol/L-1 mol/L, for example, 0.5 mol/L, 0.6 mol/L, 0.7 mol/L, 0.8 mol/L, 0.9 mol/L, 1 mol/L, etc.

In some embodiments of this aspect, the concentration of mannitol in the preservation component is 1.5%-4.5% (w/v), in mass volume ratio % (w/v), and examples include but are not limited to 1.5%, 2%, 2.5%, 3%, 3.5%, 4%, 4.5%, etc.

In some embodiments of this aspect, the concentration of glycerol in the preservation component is 2%-10% (v/v), in volume ratio %, and examples include but are not limited to 2%, 3%, 4%, 5%, 6%, 7%, 8%, 9%, 10%, etc.

In some embodiments of this aspect, the preservation component also includes urea. Concentration of urea in the preservation components may be 1%-3% (w/v), in mass volume ratio % (w/v), and examples include but are not limited to 1%, 1.5%, 2%, 2.5%, 3%, etc.

In some embodiments of this aspect, the preservation component includes (in final concentration in the preservation component): 1 mmol/L-5 mmol/L of citric acid, 0.1%-1.2% (w/v) of sodium chloride, 0.1%-1.2% (w/v) of potassium chloride, 0.8 mol/L-1 mol/L of glycine, 0.6 mol/L-1 mol/L of isoleucine, 0.5 mol/L-1 mol/L of trehalose, 1.5%-4.5% (w/v) of mannitol and 2%-10% (v/v) of glycerol.

The fifth aspect provides a combination product. If there is no special consideration, the respective components in the liquefaction components can be provided separately or in combination, and the respective components in the preservation components can also be provided separately or in combination. When provided in combination, it may be liquid, further may be solid or semi-solid. Liquid includes but is not limited to be in a form such as solution, emulsion, suspension, etc. Examples of semi-solid state include but are not limited to a gelatinous state, etc.

In some embodiments of this aspect, the respective components in the liquefaction component constitutes an integrated combined state.

In some embodiments of this aspect, the respective components in the preservation component constitutes an integrated combined state.

In some embodiments of this aspect, of the respective components of the liquefaction component, some components are present in a combined state with other components, and some components are independently present. In some preferred embodiments, the fourth aqueous solvent are independently present. In some preferred embodiments, adsorbing agent (i.e., the fourth adsorbing agent) is independently present. In some preferred embodiments, the rigid microparticles are independently present. In some preferred embodiments, two components, i.e., guaifenesin and Surfactin, are present in a combined state. In some preferred embodiments, three components, i.e., guaifenesin, Surfactin and the first strong base, are present in a combined state. In some preferred embodiments, four components, i.e., guaifenesin, Surfactin, the first strong base and the aqueous solvent (the fourth aqueous solvent), are present in a combined state. In some preferred embodiments, guaifenesin, Surfactin and the first strong base are present each independently with the aqueous solvent (the fourth aqueous solvent) in a combined state, respectively.

In some embodiments of this aspect, of the respective components of the liquefaction component, some components are present in a combined state with other components, and some components are independently present. In some preferred embodiments, component a), component b) and component c) are each independently present. In some preferred embodiments, component b) and component a) are present in a combined state, and component c) and component a) also are present in a combined state.

In a sixth aspect of this disclosure, there is provided a kit, which includes at least one of the viscous biological sample liquefying composition as described in the first aspect of this disclosure, the liquefying agent as described in the second aspect of this disclosure, the viscous biological sample processing combination product as described in the third aspect of this disclosure, the viscous biological sample liquefying agent as described in the fourth aspect of this disclosure, and the combination product for liquefying and preserving a viscous biological sample as described in the fifth aspect of this disclosure.

In some embodiments of this disclosure, the kit further includes nucleic acid processing component, further, the nucleic acid processing component includes one, two or three of a nucleic acid extraction agent, a nucleic acid amplification agent and a nucleic acid detection agent.

In some embodiments of this disclosure, the kit may contain any one or more of a releasing agent, an extraction agent, an amplification agent, and a detection agent for nucleic acids.

In some embodiments of this disclosure, the kit includes the viscous biological sample liquefying composition as described in the first aspect of this disclosure.

In some embodiments of this disclosure, the kit includes the liquefying agent in the second aspect of this disclosure.

In some embodiments of this disclosure, the kit includes the viscous biological sample processing combination product as described in the third aspect of this disclosure.

In some embodiments of this disclosure, the kit includes the viscous biological sample liquefying agent as described in the fourth aspect of this disclosure.

In some embodiments of this disclosure, the kit includes the combination product for liquefying and preserving a viscous biological sample as described in the fifth aspect of this disclosure. At this point, the kit can be denoted as a liquefaction kit, and the liquefaction component in the liquefaction kit can be used to prepare the viscous biological sample liquefying agent as described in the fourth aspect of this disclosure. After using a component in the kit to liquefy the viscous biological sample, the subsequent amplification or detection can be carried out directly without release and extraction. After liquefaction, the subsequent amplification or detection can be carried out directly, or, the amplification or detection can be carried out directly after preserving with the preservation component as necessary. The viscous biological sample liquefying agent prepared by the liquefaction kit can quickly and efficiently liquefy the viscous biological sample in a time ≤15 min (preferably ≤10 min, more preferably ≤5 min, or even ≤3 min), thereby greatly accelerating the detection efficiency of viscous biological sample. Taking sputum as an example, it takes 5 h-24 h to detect the nucleic acids in sputum by the traditional technology, but in this disclosure, by omitting the release and purification steps, the duration for detection cycle can be reduced to 1.5 hours or even shorter.

In some embodiments of this disclosure, the nucleic acids include DNA and/or RNA, and the nucleic acids have various forms, including short oligonucleotide, long oligonucleotide or polynucleotide, and further may be double stranded DNA and single stranded DNA, as well as double stranded RNA and single stranded RNA. In some specific embodiments, nucleic acids can be genes, cDNA molecules, mRNA, tRNA, rRNA, non-coding RNA molecules, etc., and fragments having a form of nucleic acids as described above, such as oligonucleotides.

In some embodiments of this disclosure, the kit satisfies any one or any suitable variety of characteristics (tk1), (tk2), (tk3), (tk4) and (tk5).

In some embodiments of this disclosure, the kit satisfies characteristic (tk1): the kit includes the viscous biological sample liquefying composition, and further includes any one or more of preservation agent, releasing agent, extraction agent, amplification agent and detection agent suitable for nucleic acids; further, the nucleic acids can include one or both of DNA and RNA.

In some embodiments of this disclosure, the kit satisfies characteristic (tk2): the kit includes a combination product for liquefying and preserving a viscous biological sample, the combination product for liquefying and preserving a viscous biological sample includes the liquefaction component and the preservation component in the viscous biological sample processing combination product as described in the third aspect of this disclosure, further, the kit further includes any one or more of the releasing agent, extraction agent, amplification agent and detection agent suitable for nucleic acids; still further, the nucleic acids include one or both of DNA and RNA.

In some embodiments of this disclosure, the kit satisfies characteristic (tk3): the kit includes a combination product for liquefying and releasing a viscous biological sample, the combination product for liquefying and preserving a viscous biological sample includes the liquefaction component and the nucleic acid releasing component in the viscous biological sample processing combination product as described in the third aspect of this disclosure, further, the kit further includes one or more of the preservation agent, extraction agent, amplification agent and detection agent suitable for nucleic acids; still further, the nucleic acids include one or both of DNA and RNA.

In some embodiments of this disclosure, the characteristic (tk3) is: the kit includes a combination product for liquefying and releasing a viscous biological sample, the combination product for liquefying and preserving a viscous biological sample includes the liquefaction component, the nucleic acid releasing component and the preservation component in the viscous biological sample processing combination product as described in the third aspect of this disclosure, further, the kit further includes one or more of the extraction agent, amplification agent and detection agent suitable for nucleic acids; still further, the nucleic acids include one or both of DNA and RNA. In some embodiments of this disclosure, the kit satisfies characteristic (tk4): the kit includes the viscous biological sample liquefying agent as described in the fourth aspect of this disclosure, further, the kit further includes one or more of preservation agent, amplification agent and detection agent suitable for nucleic acids; still further, the nucleic acids include one or both of DNA and RNA.

In some embodiments of this disclosure, the kit satisfies characteristic (tk5): the kit includes the combination product for liquefying and preserving a viscous biological sample as described in the fifth aspect of this disclosure (Corresponding to the above liquefaction kit), further, the kit includes one or both of the amplification agent and detection agent suitable for nucleic acids; still further, the nucleic acids include one or both of DNA and RNA.

In this disclosure, if there is no special consideration, the respective components in the liquefaction component can be provided separately in different packaging containers or in combination in the same packaging container, the respective components in the nucleic acid releasing component can also be provided separately in different packaging containers or in combination, and the respective components in the preservation component can also be provided separately in different packaging containers or in combination. When provided in combination, it may be liquid, further may be solid or semi-solid. The liquid includes but is not limited to forms such as solution, emulsion, suspension, etc. The examples of semi-solid state include but are not limited to a gelatinous state, etc.

In some embodiments of this disclosure, the respective components in the liquefaction component constitutes an integrated combined state.

In some embodiments of this disclosure, the respective components in the nucleic acid releasing component constitutes an integrated combined state.

In some embodiments of this disclosure, the respective components in the preservation component constitutes an integrated combined state.

In some embodiments of this disclosure, of the respective components of the liquefaction component, some components are present in a combined state with other components, and some components are independently present. In some embodiments, the rigid microparticles are independently present.

In some embodiments of this disclosure, the buffer component in the liquefaction component is independently present.

In some embodiments of this disclosure, the buffer component in the nucleic acid releasing component is independently present.

In some embodiments of this disclosure, the buffer component in the preservation component is independently present.

In some embodiments of this disclosure, the liquefaction component, nucleic acid releasing component and buffer component in the preservation component are provided by buffer reagents of the same component type. Further, the content of the components contained therein is also the same.

In some embodiments of this disclosure, of the respective components of the preservation component, some components are present in a combined state with other components, and some components are independently present. In some preferred embodiments, component a), component b) and component c) are each independently present. In some preferred embodiments, component b) and component a) are present in a combined state, and component c) and component a) are present in a combined state.

The kit in the sixth aspect of this disclosure (including but not limited to any of the aforementioned kit, including but not limited to the aforementioned liquefaction kit) further includes a label and/or an instruction.

The kit in the sixth aspect of this disclosure (including but not limited to any of the aforementioned kit, including but not limited to the aforementioned liquefaction kit) further includes a container for encapsulating the respective components.

In some embodiments of this disclosure, the aforementioned liquefaction kit includes liquefaction component, optional preservation component and optional amplification-related component. The liquefaction component can refer to the components of the viscous biological sample liquefying agent in the fourth aspect of this disclosure. In some embodiments of this disclosure, the liquefaction component includes guaifenesin, the first strong base, Surfactin, Na⁺ and/or K⁺, the fourth aqueous solvent, the optional adsorbing agent (the fourth adsorbing agent) and the optional rigid microparticles. The definition, type, content, optimization, examples, etc. of each component include but are not limited to those described in the fourth aspect, which will not be repeated here. The fourth aqueous solvent can be directly preset in the kit (packaged separately or in combination with other components), or can be prepared by the user himself.

In some embodiments of this disclosure, the liquefaction component in the aforementioned liquefaction kit includes:
component L1): guaifenesin;
component L2): the first strong base;
component L3): Surfactin;
component L4): optional sodium source and/or potassium source (for providing Na⁺ and/or K⁺);
component L5): optional first aqueous solvent;
component L6): optional adsorbing agent (the fourth adsorbing agent); and
component L7): optional rigid microparticles;
The liquefaction component in the liquefaction kit can be used to prepare the viscous biological sample liquefying agent as described in the fourth aspect of this disclosure.

In some embodiments of this disclosure, the first strong base is sodium hydroxide and/or potassium hydroxide.

In some embodiments of this disclosure, the first adsorbing agent, the second adsorbing agent, the third adsorbing agent and the fourth adsorbing agent are each independently Chelex resin.

In some embodiments of this disclosure, the rigid microparticles are one or more selected from zirconia, silicon nitride, ceramsite, hard stainless steel, hard tungsten carbide, sintered corundum and agate, etc.

In some embodiments of this disclosure, the rigid microparticles is sphere, near sphere, ellipsoid, column, rod, polyhedron or irregular shape.

In some embodiments of this disclosure, the average particle size of the rigid microparticles is 0.01 mm-10 mm.

In some embodiments of this disclosure, the fourth aqueous solvent is water or a fourth buffer component.

In some embodiments of this disclosure, the preservation component in the liquefaction kit includes a third buffer component, an osmotic pressure regulating component, and one, two or three of trehalose, mannitol, and glycerol. In some embodiments of this disclosure, the amplification-related components in the liquefaction kit include one, two or three of a nucleic acid extraction agent, a nucleic acid amplification agent and a nucleic acid detection agent.

In some embodiments, the respective components of the liquefaction components in any kit of the sixth aspect can be packaged separately or packaged together in combination.

In some embodiments, in any kit containing preservation component of the sixth aspect, the respective components of the preservation component can be packaged separately or packaged together in combination.

In some embodiments, in any kit of the sixth aspect, of the respective components of the liquefaction component, some components are packaged together with other components in the same container, and some components are packaged independently in one container. In some preferred embodiments, the corresponding aqueous solvent (such as the fourth aqueous solvent in the liquefaction kit) is packaged separately in a container. In some preferred embodiments, the corresponding adsorbing agent (such as the fourth adsorbing agent in the liquefaction kit) is packaged separately in a container. In some preferred embodiments, the corresponding rigid microparticles are packaged independently in a container.

In some preferred embodiments, in the liquefaction kit, the two components, i.e., guaifenesin and Surfactin, are packaged in a same container. In some preferred embodiments, the three components, i.e., guaifenesin, Surfactin and the first strong base, are packaged in a same container. In some preferred embodiments, the four components, i.e., guaifenesin, Surfactin, the first strong base and aqueous solvent (the fourth aqueous solvent), are packaged in a same container. In some preferred embodiments, the guaifenesin, Surfactin and the first strong base are each independently packaged with the aqueous solvent (the fourth aqueous solvent), respectively, that is, the liquefaction kit provides guaifenesin solution, Surfactin solution and the first strong base solution, each of which packaged independently.

In some embodiments of this disclosure, guaifenesin and the strong base (the first strong base) can be packaged separately. At this point, the amount of strong base added can be flexibly controlled according to the characteristics of the viscous biological sample, so that the pH value of the liquefaction system can be flexibly controlled to better adapt to the differential characteristics between different samples. Similarly, in some embodiments, the content of guaifenesin can further be flexibly controlled to facilitate more accurate control of the liquefaction of trace samples.

In some embodiments, in any kit containing preservation component of the sixth aspect, of the respective components of the preservation component, some components are packaged in a same container with other components, and some components are independently packaged in one container. In some preferred embodiments, component a), component b) and component c) are packaged separately. In some preferred embodiments, component b) and component a) are packaged in a same container, and component c) and component a) are also packaged in a same container, that is, buffer solution containing component b) and buffer solution containing component c) independently packaged separately are provide.

In a seventh aspect of this disclosure, there is provided a method for liquefying a viscous biological sample, which includes the following steps: S100, mixing the viscous biological sample with a liquefaction component to obtain a first mixture; and then S200, incubating the first mixture;
wherein,

In some embodiments (denoted as scheme S-I), when the liquefaction component is the viscous biological sample liquefying composition as described in the first aspect of this disclosure or the liquefying agent as described in the second aspect of this disclosure, a second mixture is obtained after incubation;

In other embodiments (denoted as scheme S-II), when the liquefaction component is selected from the viscous biological sample liquefying agent as described in the fourth aspect of this disclosure, the liquefied product is obtained after incubation.

In some embodiments of this disclosure, the viscous biological sample is sputum, cervical mucus, nasopharyngeal swab or oral swab.

In some embodiments of this disclosure, a method for liquefying a viscous biological sample is provided, which includes the following steps:
S100a: mixing the viscous biological sample with the liquefaction component in Scheme S-I as described above to obtain a first mixture;
S200a: incubating the first mixture to obtain a second mixture.

In some embodiments of this disclosure, a method for liquefying a viscous biological sample is provided, which includes the following steps:
S100b: mixing the viscous biological sample with the liquefaction component in Scheme S-II as described above to obtain a first mixture;
S200b: incubating the first mixture to obtain a liquefied product.

In some embodiments of this disclosure, the incubation conditions satisfy one or more of the following characteristics (tb1), (tc1), (tc2), (tc3), (tc4) and (tc5).

In some embodiments of this disclosure, the incubation conditions satisfy the characteristic (tc1): the pH is greater than or equal to 10, further preferably 10-14, still further preferably 10-12 or 12-14, yet still further preferably 10, 10.5, 11, 11.5, 12, 12.5, 13, 13.5 or 14.

In some embodiments of this disclosure, the incubation conditions satisfy the characteristic (tc2): the concentration of guaifenesin in the first mixture is 1 mmol/L-1 mol/L; preferably 20 mmol/L-1 mol/L; further preferably 20 mmol/L-500 mmol/L; further preferably 20 mmol/L-400 mmol/L; further preferably 20 mmol/L-200 mmol/L; further preferably 20 mmol/L-150 mmol/L; further preferably 50 mmol/L-400 mmol/L; further preferably 50 mmol/L-200 mmol/L; further preferably 50 mmol/L-150 mmol/L; further preferably 50 mmol/L, 100 mmol/L, 150 mmol/L or 200 mmol/L.

In some embodiments of this disclosure, the incubation conditions satisfy the characteristic (tc3): mixing by stirring or shaking.

In some embodiments of this disclosure, the incubation conditions satisfy the characteristic (tc4): the incubation temperature is selected from 18-35° C., may be 20-35° C., further may be 25-35° C., further may be 18-30° C., further may be 20-30° C., further may be 25-30° C., or further may be 18° C., 20° C., 21° C., 22° C., 23° C., 24° C., 25° C., 26° C., 27° C., 28° C., 29° C., 30° C., 31° C., 32° C., 33° C., 34° C. or 35° C.

In some embodiments of this disclosure, the incubation conditions satisfy the characteristic (tc5): the incubation temperature is less than 30 min, may be less than 15 min, further may be 2 min-15 min, further may be 3 min-15 min, further may be 2 min-10 min, further may be 3 min-10 min, further may be 2 min-8 min, further may be 3 min-8 min, further may be 2 min-6 min, further may be 3 min-6 min, further may be 2 min-5 min, further may be 3 min-5 min, or further may be 2 min, 3 min, 4 min, 5 min, 6 min, 7 min, 8 min, 9 min, 10 min or 15 min.

The above steps S100 and S200 enable the liquefaction of the viscous biological sample and reduce viscosity of the sample.

In some embodiments of this disclosure, the pH value of the first mixture is greater than or equal to 10, for example, 11, 11.5, 12, 12.5, 13, 13.5, 14, 14.5, 15, preferably 12-14.

In some embodiments of this disclosure, the concentration of guaifenesin in the first mixture is 1 mmol/L-1 mol/L, for example, 5 mmol/L, 10 mmol/L, 50 mmol/L, 60 mmol/L, 70 mmol/L, 80 mmol/L, 90 mmol/L, 100 mmol/L, 110 mmol/L, 120 mmol/L, 130 mmol/L, 140 mmol/L, 150 mmol/L, 200 mmol/L, 300 mmol/L, 400 mmol/L, 500 mmol/L, 600 mmol/L, 700 mmol/L, 800 mmol/L or 900 mmol/L.

In some embodiments of this disclosure, the incubation temperature is 18-35° C., or further may be selected from 20° C., 21° C., 22° C., 23° C., 24° C., 25° C., 26° C., 27° C., 28° C., 29° C., 30° C., 31° C., 32° C., 33° C., and 34° C.

In some embodiments of this disclosure, the incubation time is greater than or equal to 5 min, and further may be selected from 6 min, 7 min, 8 min, 9 min, 10 min, 15 min, 20 min, 30 min, etc.

In some embodiments of this disclosure, the incubation time is less than 15 min. Examples of incubation time include, but are not limited to, any of the following durations, or the duration interval between any two of the followings: 2 min, 3 min, 4 min, 5 min, 6 min, 7 min, 8 min, 9 min, 10 min, 15 min, etc. Examples of the duration interval between any two of the followings are: 2 min-15 min, 3 min-15 min, 3 min-10 min, etc.

In some embodiments, the mixing is carried out by stirring or shaking.

In an eighth aspect of this disclosure, there is provided a method for processing a viscous biological sample, which includes the step of liquefying the viscous biological sample by adopting the method for liquefying a viscous biological sample as described in the seventh aspect of this disclosure.

In some embodiments of this disclosure, in the following embodiments of this aspect, the viscous biological sample can be sputum, cervical mucus, nasopharyngeal swab or oral swab.

In some embodiments of this disclosure, the method for processing the viscous biological sample is selected from any of the following methods: liquefaction and preservation method, liquefaction and release method, extraction method, amplification method and detection method.

In some embodiments of this disclosure, a method for liquefying and preserving a viscous biological sample is provided, which includes the following steps:
S100: mixing the viscous biological sample with the liquefaction component to obtain a first mixture;
S200: incubating the first mixture;
S300: mixing the incubated first mixture with the preservation component (referring to the third aspect of this disclosure) as described above to obtain a liquefied sample-preserving solution.

In some embodiments of this disclosure, the liquefaction and preservation method includes the following steps: mixing the second mixture or the liquefied product with the preservation component in the viscous biological sample processing combination product as described in the third aspect of this disclosure to obtain the liquefied sample-preserving solution; preferably, the volume ratio of the preservation component to the second mixture or the liquefied product is 1:(2-4), more preferably 1:2, 1:2.5, 1:3, 1:3.5 or 1:4.

In some embodiments of this disclosure, a method for liquefying and preserving a viscous biological sample is provided, which includes the following steps:
S100a: mixing the viscous biological sample with the liquefaction component of Scheme S-I as described above to obtain a first mixture;
S200a: incubating the first mixture to obtain a second mixture;
S300a: mixing the second mixture with the preservation component (which can refer to the third aspect of this disclosure) as described above to obtain a liquefied sample-preserving solution I.

In some embodiments of this disclosure, a method for liquefying and preserving a viscous biological sample is provided, which includes the following steps:
S100b: mixing the viscous biological sample with the liquefaction component in Scheme S-II as described above to obtain a first mixture;
S200b: incubating the first mixture to obtain a liquefied product;
S300b: mixing the liquefied product with the preservation component (which can refer to the third aspect of this disclosure) as described above to obtain a liquefied sample-preserving solution II.

The above step S100 (e.g. S100a or S100b) enables liquefaction and reduces the viscosity of the sample.

When performing step S200 (e.g. S200a or S200b), the preservation component can be added together, or different preservation components can be added separately.

In this disclosure, the pH of the system during liquefaction is preferably alkaline, preferably to the extent sufficient to provide a strong alkali environment with pH greater than or equal to 10.

In some embodiments of this disclosure, guaifenesin and the strong base can be packaged separately. At this point, the amount of strong base added and thus the pH value of the liquefaction system can be flexibly controlled according to the characteristics of the viscous biological sample, in order to better adapt to differential characteristics between different samples.

In some embodiments of this disclosure, the pH value of the first mixture is greater than or equal to 10, for example, pH 10, pH 11, pH 11.5, pH 12, pH 12.5, pH 13, pH 13.5, pH 14, pH 14.5, pH 15, preferably pH 12-pH 14. By adjusting the pH value of the first mixture, the pH value during incubation in step S200 (e.g. S200a or S200b) can be controlled.

Similarly, in some embodiments of this disclosure, the content of guaifenesin can also be flexibly controlled to facilitate more accurate control of the liquefaction of trace samples. In some embodiments, the concentration of the guaifenesin in the first mixture (i.e., the working concentration of guaifenesin) is 1 mmol/L-1 mol/L, for example, 5 mmol/L, 10 mmol/L, 50 mmol/L, 60 mmol/L, 70 mmol/L, 80 mmol/L, 90 mmol/L, 100 mmol/L, 110 mmol/L, 120 mmol/L, 130 mmol/L, 140 mmol/L, 150 mmol/L, 200 mmol/L, 300 mmol/L, 400 mmol/L, 500 mmol/L, 600 mmol/L, 700 mmol/L, 800 mmol/L or 900 mmol/L.

In this disclosure, liquefaction is carried out without harsh temperature conditions. For example, it can be operated at room temperature.

In some embodiments of this disclosure, the incubation temperature is 18° C.-35° C. Examples of incubation temperatures include, but are not limited to: 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., etc.

During liquefaction, the incubation time can be reasonably controlled according to the sample size, sample characteristics and other factors, so as to liquefy completely within a reasonable time.

In some embodiments of this disclosure, from the perspective of clinical operability, 5 min-30 minis relatively better.

In some embodiments of this disclosure, the incubation time is greater than or equal to 5 min. Examples of the incubation time include, but are not limited to: 5 min, 6 min, 7 min, 8 min, 9 min, 10 min, 15 min, 20 min, 30 mm or longer.

In some embodiments of this disclosure, the incubation time is less than 15 mm. Examples of incubation time include, but are not limited to, any of the following durations, or the duration interval between any two of the followings: 2 min, 3 min, 4 min, 5 min, 6 min, 7 min, 8 min, 9 min, 10 min, 15 min, etc. Examples of the duration interval between any two of the followings are: 2 min-15 min, 3 min-15 min, 3 min-10 min, etc.

In some embodiments of this disclosure, the mixing is carried out by stirring or shaking.

With regard to the dosage of the preservation component and the second mixture or the liquefied product, the pH value of the liquefied sample is adjusted to a mild range (e.g. pH 6-pH 8) to facilitate the stable preservation of nucleic acid substance.

In some embodiments of this disclosure, the volume ratio of the preservation component to the second mixture is 1:(2-4), and further may be selected from 1:2, 1:2.5, 1:3, 1:3.5, 1:4, etc.

In some embodiments of this disclosure, the volume ratio of the preservation component to the liquefied product is 1:(2-4), and further may be selected from 1:2, 1:2.5, 1:3, 1:3.5, 1:4, etc.

The mixing method in step S100 (e.g. S100a or S100b) is not particularly limited, provided that the sample is in sufficient contact with the liquefaction component and can be liquefied successfully.

The mixing method in step S300 (e.g. S300a or S300b) is not particularly limited, provided that the pH value of the system can be successfully adjusted to a predetermined range and the system can make a full contact with the osmotic pressure regulating components, etc., to play a protective role on nucleic acid substance.

In some embodiments of this disclosure, step S100 (e.g. S100A or S100b) is mixed by stirring or shaking.

In some embodiments of this disclosure, the viscous biological sample is sputum, cervical mucus, nasopharyngeal swab or oral swab.

In some embodiments, the viscous biological sample includes sputum or cervical mucus.

In some embodiments of this disclosure, the liquefaction and release method includes the following steps S400: processing the second mixture or the liquefied sample-preserving solution I with a nucleic acid releasing agent to obtain a release processing solution;
preferably, the nucleic acid releasing agent is selected from the nucleic acid releasing component in the viscous biological sample processing combination product in the third aspect of this disclosure.

In this disclosure, after the second mixture or the liquefied sample-preserving solution I is treated with a nucleic acid releasing agent, the nucleic acids in the second mixture or the liquefied sample-preserving solution I is released.

The term “the release of nucleic acids” as used in the eighth aspect of this disclosure is consistent with the foregoing, and refers to the release of nucleic acids from the sample and being at a state that can be extracted/enriched/purified/detected. The release of nucleic acids is usually accompanied by cell lysis and the physical separation of impurity components such as protein, lipid, polysaccharide, etc., from nucleic acid components by simple methods such as centrifugation, etc., which is different from the situation at physiological conditions, and nucleic acids can be directly detected by some detection reagents.

In some embodiments of this disclosure, the second mixture is processed with a nucleic acid releasing agent to obtain a release processing solution I; at this point, step S400 is denoted as S400a.

In some embodiments of this disclosure, the volume ratio of the second mixture to the nucleic acid releasing agent is 1:(0.5-1.5). The volume ratio of the second mixture to the nucleic acid releasing agent further may be 1:0.5, 1:0.6, 1:0.7, 1:0.8, 1:0.9, 1:1, 1:1.1, 1:1.2, 1:1.3, 1:1.4 or 1:1.5.

In some embodiments, a method for liquefying and releasing a viscous biological sample is provided, which includes the following steps:
S100a: mixing the viscous biological sample with the liquefaction component in Scheme S-I as described above to obtain a first mixture;
S200a: incubating the first mixture to obtain a second mixture;
S400a: mixing the second mixture with the nucleic acid releasing agent (e.g. the nucleic acid releasing component in the third aspect) as described above and releasing the nucleic acids in the sample to obtain the release processing solution I.

In some embodiments of this disclosure, the liquefied sample-preserving solution I is processed with a nucleic acid releasing agent to obtain a release processing solution III; at this point, step S400 is denoted as S400b.

In some embodiments of this disclosure, the volume ratio of the liquefied sample-preserving solution I to the nucleic acid releasing agent is 1:(0.5-1.5). The volume ratio of the liquefied sample-preserving solution I to the nucleic acid releasing agent further may be 1:0.5, 1:0.6, 1:0.7, 1:0.8, 1:0.9, 1:1, 1:1.1, 1:1.2, 1:1.3, 1:1.4 or 1:1.5. In some embodiments, a method for liquefying and releasing a viscous biological sample is provided, which includes the following steps:
S100a: mixing the viscous biological sample with the liquefaction component in Scheme S-I as described above to obtain a first mixture;
S200a: incubating the first mixture to obtain a second mixture;
S300a: mixing the second mixture with the preservation component (may be selected from the preservation component in the third aspect of this disclosure) as described above to obtain a liquefied sample-preserving solution I;
S400c: mixing the liquefied sample-preserving solution I with the nucleic acid releasing agent (e.g. the nucleic acid releasing component in the third aspect) as described above and releasing the nucleic acids in the sample to obtain the release processing solution III.

In this disclosure, the above step S100 (e.g. S100a or S100b) can provide a suitable pH environment for the subsequent liquefaction.

In this disclosure, the pH of the system during liquefaction is preferably alkaline, preferably to the extent sufficient to provide a strong alkali environment with pH greater than or equal to 10.

In some embodiments of this disclosure, guaifenesin and the first strong base can be packaged separately. At this point, the amount of the first strong base added and thus the pH value of the liquefaction system can be flexibly controlled according to the characteristics of the viscous biological sample, to better adapt to the differential characteristics between different samples.

In some embodiments of this disclosure, guaifenesin and the strong base can be packaged separately. At this point, the amount of the strong base added and thus the pH value of the liquefaction system can be flexibly controlled according to the characteristics of the viscous biological sample, to better adapt to the differential characteristics between different samples.

Similarly, in some embodiments of this disclosure, the content of guaifenesin can also be flexibly controlled to facilitate more accurate control of the liquefaction of trace samples.

In some embodiments of this disclosure, the pH value of the first mixture is greater than or equal to 10, for example, pH 10, pH 11, pH 11.5, pH 12, pH 12.5, pH 13, pH 13.5, pH 14, pH 14.5, pH 15, preferably pH 12-pH 14. By adjusting the pH value of the first mixture, the pH value during incubation in the step ii) can be controlled. In some embodiments of this disclosure, the concentration of the guaifenesin in the first mixture (i.e., the working concentration of guaifenesin) is 1 mmol/L-1 mol/L, for example, 1 mmol/L, 5 mmol/L, 10 mmol/L, 50 mmol/L, 60 mmol/L, 70 mmol/L, 80 mmol/L, 90 mmol/L, 100 mmol/L, 110 mmol/L, 120 mmol/L, 130 mmol/L, 140 mmol/L, 150 mmol/L, 200 mmol/L, 300 mmol/L, 400 mmol/L, 500 mmol/L, 600 mmol/L, 700 mmol/L, 800 mmol/L, 900 mmol/L, 1 mol/L, etc.

The above step S200 (e.g. S200a or S200b) enables liquefaction and reduces the viscosity of the sample.

In this disclosure, liquefaction is carried out without harsh temperature conditions, for example, it can be operated at room temperature.

In some embodiments of this disclosure, the incubation temperature is 18° C.-35° C. Examples of incubation temperatures include, but are not limited to: 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., etc.

In some embodiments of this disclosure, the incubation time is greater than or equal to 5 min. Examples of the incubation time include, but are not limited to: 5 min, 6 min, 7 min, 8 min, 9 min, 10 min, 15 min, 20 min, 30 min or longer.

In some embodiments of this disclosure, the incubation time is less than 15 min. Examples of incubation time include, but are not limited to, any of the following durations, or the duration interval between any two of the followings: 2 min, 3 min, 4 min, 5 min, 6 min, 7 min, 8 min, 9 min, 10 min, 15 min, etc. Examples of the duration interval between any two of the followings are: 2 min-15 min, 3 min-15 min, 3 min-10 min, etc.

In this disclosure, during liquefaction, the incubation time can be reasonably controlled according to the sample size, sample characteristics and other factors, so as to liquefying completely within a reasonable time.

In some embodiments of this disclosure, from the perspective of clinical operability, 5 min-30 min is relatively better.

In some embodiments of this disclosure, in the liquefaction and release method, step S300a is also carried out after step S200a: mixing and preserving the incubated first mixture (i.e., the second mixture) with the preservation component as described above to obtain the aforementioned liquefied sample-preserving solution I. That is, the viscous biological sample after liquefying is preserved with the preservation component, and then the nucleic acid releasing step is carried out as necessary. At this point, step S400 corresponds to S400c: mixing the liquefied sample-preserving solution I as described above with the nucleic acid releasing agent as described above and releasing the nucleic acids in the sample to obtain the release processing solution III.

In some embodiments of this disclosure, a method for liquefying and releasing the nucleic acids in a viscous biological sample is provided, which includes the following steps:
liquefying and preserving the viscous biological sample using the liquefaction and preservation method as described above; and
releasing the nucleic acids in the viscous biological sample using nucleic acid releasing agent.

In some embodiments of this disclosure, the volume ratio of the preservation component to the first mixture is 1:(2-4), and further may be selected from 1:2, 1:2.5, 1:3, 1:3.5, 1:4, etc.

The above step S400 (e.g. S400a, S400c, etc.) enables the release of nucleic acids.

In some embodiments of this disclosure, the nucleic acid releasing component is the composition as shown in component ii), which can effectively release DNA.

In some embodiments of this disclosure, the nucleic acid releasing component is the composition shown in component i), which can effectively release both DNA and RNA.

In some embodiments of this disclosure, the nucleic acid releasing component is a composition comprising component i) and component ii), which can effectively release both DNA and RNA.

In some embodiments of this disclosure, a single mixed system of the nucleic acid releasing component is provided in an independent package.

In some embodiments of this disclosure, the volume ratio of the second mixture to the nucleic acid releasing component in the step S400a is 1:(0.5-1.5).

In some embodiments of this disclosure, the volume ratio of the liquefied sample-preserving solution I to the nucleic acid releasing component in the step S400c is 1:(0.5-1.5).

In this disclosure, the mixing mode in step S100 (e.g. S100a or S100b) is not particularly limited, provided that the sample is in full contact with the liquefaction component and can be liquefied successfully.

In this disclosure, the mixing mode in step S400 (e.g. S400a or S400c) is also not particularly limited, provided that the release of nucleic acids can be successfully achieved.

In some embodiments of this disclosure, in the step S100 and/or S400, the fluid is mixed by stirring or shaking. In some embodiments of this disclosure, the viscous biological sample is sputum, cervical mucus, nasopharyngeal swab or oral swab of this disclosure.

In some embodiments of this disclosure, the viscous biological sample is selected from the group consisting of sputum and cervical mucus.

In some embodiments of this disclosure, the viscous biological sample is sputum and/or cervical mucus.

In some embodiments of this disclosure, the extraction method includes the following step S500: extracting the nucleic acids in the release processing solution with a nucleic acid extraction agent to obtain the extraction processing solution. The release processing solution may be the release processing solution I or the release processing solution III.

The term “extraction of nucleic acids” refers to a process of separating the released nucleic acids from impurities, which is usually accompanied by the purification of nucleic acid components. For example, centrifugation, enrichment using magnetic beads, stratification of sample components caused by chemical reagent processing, etc. In some embodiments, a method for extracting nucleic acids from viscous biological sample is provided, including:
liquefying the viscous biological sample and releasing the nucleic acids in the viscous biological sample using the liquefaction and release method as described above;
extracting the released nucleic acids with the nucleic acid extraction agent. In some embodiments, there is provided a method for amplifying the nucleic acids in the viscous biological sample, including:
liquefying the viscous biological sample and releasing the nucleic acids in the viscous biological sample using the liquefaction and release method as described above;
optionally, extracting the released nucleic acids with the nucleic acid extraction agent; and
amplifying the nucleic acids using the nucleic acid amplification agent.

When the term “amplifying or amplification” occurs collectively in the context of the term “nucleic acid(s)”, it refers to production of multiple copies of polynucleotide or a portion of a polynucleotide, usually starting with a small number of polynucleotide (for example, as few as a single polynucleotide molecule), where the amplification product or amplicon is usually detectable. The amplification of polynucleotide includes a variety of chemical and enzymatic methods. In the process of polymerase chain reaction (PCR), rolling circle amplification (RCA) or ligase chain reaction (LCR), production of multiple copies of DNA from one or several copies of target DNA or template DNA molecules is an amplification mode. Amplification is not limited to the strict replication of the starting molecule. For example, production of multiple cDNA molecules from a limited amount of RNA in the sample using reverse transcription RT-PCR is an amplification mode. In addition, the production of multiple RNA molecules from a single DNA molecule during the transcription process is also an amplification mode. Amplification can be used to build a library before sequencing.

In some embodiments, the amplification method includes the following step S600: amplifying the nucleic acids in the release processing solution, the extraction processing solution, the liquefied product or the liquefied sample-preserving solution II with a nucleic acid amplification agent to prepare an amplification mixture. The release processing solution may be the release processing solution I or the release processing solution III.

In some embodiments, the amplification method includes the following step S600a: amplifying the release processing solution using the nucleic acid amplification agent to prepare an amplification mixture. The release processing solution may be the release processing solution I or the release processing solution III.

In some embodiments, the amplification method includes the following step S600b: amplifying the extraction processing solution using the nucleic acid amplification agent to prepare an amplification mixture.

In some embodiments, the amplification method includes the following step S600c: amplifying the liquefied product using the nucleic acid amplification agent to prepare an amplification mixture. In some embodiments, the amplification method includes the following step S600d: amplifying the liquefied sample-preserving solution II using the nucleic acid amplification agent to prepare an amplification mixture. The sample is processed with the viscous biological sample liquefying agent in the fourth aspect of this disclosure or the combination product for liquefying and preserving a viscous biological sample in the fifth aspect of this disclosure or the liquefaction kit in the sixth aspect of this disclosure, and the processed product can be amplified directly without extraction.

In some embodiments of this disclosure, a method for amplifying nucleic acids in the viscous biological sample is provided, which includes the following steps:
S10: providing a viscous biological sample;
S20: liquefying the viscous biological sample with a liquefying agent to obtain a liquefied product; and
S60: amplifying the target nucleic acids in the liquefied product with a nucleic acid amplification agent; Wherein, the liquefying agent is selected from the viscous biological sample liquefying agent as described in the fourth aspect of this disclosure, or is prepared by using the liquefaction component in the liquefaction kit as described in the sixth aspect of the invention.

In some embodiments of this disclosure, the viscous biological sample can be liquefied quickly and efficiently with the liquefying agent in less than or equal to 15 min (preferably less than or equal to 10 min, more preferably less than or equal to 5 min, or even less than or equal to 3 min), thereby greatly accelerating the detection efficiency of the viscous biological sample. Taking sputum sample as an example, it often takes 5 h-24 h to detect nucleic acids in sputum by the traditional technology, but 1.5 hours or even shorter by this application.

The definition of “viscous biological sample” is consistent with the foregoing herein.

In some embodiments of this disclosure, the viscous biological sample is sputum, cervical mucus, nasopharyngeal swab or oral swab.

In some embodiments of this disclosure, in step S20, liquefying the viscous biological sample includes the following steps:
S100b: mixing the viscous biological sample with the liquefying reagent to obtain a first mixture; wherein, the liquefying agent is selected from the viscous biological sample liquefying agent as described in the fourth aspect of this disclosure; and
S200b: incubating the first mixture to obtain a liquefied product.

In some embodiments of this disclosure, the nucleic acids include DNA and/or RNA in various forms, including short oligonucleotide, long oligonucleotide or polynucleotide. The nucleic acids can also be double stranded DNA and single stranded DNA, as well as double stranded RNA and single stranded RNA. In some specific embodiments, the nucleic acids can be genes, cDNA molecules, mRNA, tRNA, rRNA, non-coding RNA molecules, etc., and fragments in the form of nucleic acids such as oligonucleotides.

When the term “amplifying or amplification” occurs collectively in the context of the term “nucleic acid(s)”, it refers to production of multiple copies of polynucleotide or a portion of a polynucleotide, usually starting with a small number of polynucleotide (for example, as few as a single polynucleotide molecule), where the amplification product or amplicon is usually detectable. The amplification of polynucleotide includes a variety of chemical and enzymatic methods. In the process of polymerase chain reaction (PCR), rolling circle amplification (RCA) or ligase chain reaction (LCR), production of multiple copies of DNA from one or several copies of target DNA or template DNA molecules is an amplification mode. Amplification is not limited to the strict replication of the starting molecule. For example, production of multiple cDNA molecules from a limited amount of RNA in the sample using reverse transcription RT-PCR is an amplification mode. In addition, the production of multiple RNA molecules from a single DNA molecule during the transcription process is also an amplification mode.

In some embodiments of this disclosure, the concentration of guaifenesin in the first mixture is 20 mmol/L-1 mol/L. Examples of concentrations include, but are not limited to, any one of or a concentration interval between any two of the following concentrations: 20 mmol/L, 50 mmol/L, 60 mmol/L, 70 mmol/L, 80 mmol/L, 90 mmol/L, 100 mmol/L, 110 mmol/L, 120 mmol/L, 130 mmol/L, 140 mmol/L, 150 mmol/L, 200 mmol/L, 300 mmol/L, 400 mmol/L, 500 mmol/L, 600 mmol/L, 700 mmol/L, 800 mmol/L, 900 mmol/L, 1 mol/L, etc. In some embodiments, an example of a concentration interval between any two concentrations is as follows: 20 mmol/L-500 mmol/L, 80 mmol/L-120 mmol/L, etc.

In some embodiments of this disclosure, the concentration of Surfactin in the first mixture is 20 mmol/L-0.2 mol/L, further may be 20 mmol/L-0.15 mol/L. Examples of concentrations include, but are not limited to, any one of or a concentration interval between any two of the following concentrations: 20 mmol/L, 30 mmol/L, 40 mmol/L, 50 mmol/L, 60 mmol/L, 70 mmol/L, 80 mmol/L, 90 mmol/L, 100 mmol/L, 110 mmol/L, 120 mmol/L, 130 mmol/L, 140 mmol/L, 150 mmol/L, 200 mmol/L, etc. In some embodiments, an example of a concentration interval between any two concentrations is as follows: 20 mmol/L-150 mmol/L, 30 mmol/L-60 mmol/L, etc. In some embodiments of this disclosure, the volume ratio of liquefying reagent to viscous biological sample is (1-5):1, further may be (2-5):1, further may be (2-4):1, specifically for example 3:1, 4:1, 5:1, etc.

The concentration and dosage ratio of guaifenesin to Surfactin in the first mixture can further refer to the concentration and dosage ratio of guaifenesin to Surfactin in the viscous biological sample liquefying agent of the first aspect of this disclosure.

In this disclosure, the liquefaction is carried out without harsh temperature conditions, for example, it can be operated at room temperature.

In some embodiments of this disclosure, the pH of the incubation system of greater than or equal to 10 can be achieved by controlling the pH of the first mixture. Examples of pH during incubation are for example, 11, 11.5, 12, 12.5, 13, 13.5, 14, 14.5, 15, preferably pH 12-pH 14. Examples of pH of the incubation system are further for example, 10-12, etc.

In some embodiments of this disclosure, the incubation temperature is 18° C.-35° C. Examples of incubation temperatures include, but are not limited to: 18° C., 20° C., 21° C., 22° C., 23° C., 24° C., 25° C., 26° C., 27° C., 28° C., 29° C., 30° C., 31° C., 32° C., 33° C., 34° C., 35° C., etc.

During liquefaction, the incubation time can be reasonably controlled according to the sample size, sample characteristics and other factors, so as to achieve full liquefaction within a reasonable time.

In some embodiments of this disclosure, the incubation time is less than 15 min. Examples of incubation time include, but are not limited to, any of the following durations, or the duration interval between any two of the followings: 2 min, 3 min, 4 min, 5 min, 6 min, 7 min, 8 min, 9 min, 10 min, 15 min, etc. Examples of the duration interval between any two of the followings are: 2 min-15 min, 3 min-15 min, 3 min-10 min, etc.

In some embodiments of this disclosure, a method for detecting the nucleic acids in the viscous biological sample is provided, which includes:
liquefying the viscous biological sample and releasing the nucleic acids in the viscous biological sample using the liquefaction and release method as described above;
optionally, extracting the released nucleic acids with the nucleic acid extraction agent; and
optionally, using the nucleic acid amplification agent to amplify the nucleic acids;
using the nucleic acid detection agent to detect the nucleic acids.

In some embodiments of this disclosure, the detection method includes the following step S700: detecting the nucleic acids in the liquefied product, the liquefied sample-preserving solution II, the release processing solution, the extraction processing solution or the amplification mixture with a nucleic acid detection agent; and the detection method is for non-diagnostic and therapeutic purposes, or for at least one of diagnostic and therapeutic purposes.

In some embodiments of this disclosure, a method for detecting the nucleic acids in the viscous biological sample is provided, which includes the following steps:
S10: providing a viscous biological sample;
S20: liquefying the viscous biological sample with a liquefying agent to obtain a liquefied product; and
S70: detecting the target nucleic acids in the liquefied product with a nucleic acid detection agent;
Wherein, the liquefying agent is selected from the viscous biological sample liquefying agent as described in the fourth aspect of this disclosure, or is prepared by using the liquefaction component in the liquefaction kit as described in the sixth aspect of the invention.

The definition of viscous biological sample is consistent with the first aspect of this disclosure.

In some embodiments of this disclosure, the viscous biological sample is sputum, cervical mucus, nasopharyngeal swab or oral swab.

The definitions of steps S10 and S20 are consistent with the foregoing.

Nucleic acid detection methods can rely on or not rely on nucleic acid amplification. As used herein, the term “nucleic acid detection method” refers to any method for determining the nucleotide composition of the target nucleic acids, including but not limited to DNA sequencing method, probe hybridization method, structure specific cleavage assay (e.g., INVADER assay, (Hologic, Inc.), which is as described in, for example, the U.S.[0702] U.S. Pat. Nos. 5,846,717, 5,985,557, 5,994,069, 6,001,567, 6,090,543 and 6,872,816; Lyamichev et al., [0703] Nat. Biotech., 17:292(1999), Hall et al., PNAS, USA, 97:8272(2000), and US 2009/0253142, each of which is incorporated herein by reference in their entireties for all purposes); Enzyme mismatch cleavage method (e.g., Variagenics, U.S. Pat. Nos. 6,110,684, 5,958,692, 5,851,770, which are incorporated herein by reference in their entireties); Polymerase chain reaction (PCR) as described above; Branch hybridization method (e.g., Chiron, U.S. Pat. Nos. 5,849,481, 5,710,264, 5,124,246 and 5,624,802, which are incorporated herein by reference in their entireties); Rolling circle replication (e.g., U.S. Pat. Nos. 6,210,884, 6,183,960 and 6,235,502, which are incorporated herein by reference in their entireties); NASBA (e.g., U.S. Pat. No. 5,409,818, which is incorporated herein by reference in their entireties); Molecular beacons (e.g., U.S. Pat. No. 6,150,097, which is incorporated herein by reference in their entireties); E-Sensor technology (Motorola, U.S. Pat. Nos. 6,248,229, 6,221,583, 6,013,170 and 6,063,573, which are incorporated herein by reference in their entireties); Cycling probe technology (e.g., U.S. Pat. Nos. 5,403,711, 5,011,769 and 5,660,988, which are incorporated herein by reference in their entireties); Dade Behring signal amplification method (e.g., U.S. Pat. Nos. 6,121,001, 6,110,677, 5,914,230, 5,882,867 and 5,792,614, which are incorporated herein by reference in their entireties); Ligase chain reaction (e.g., Baranay Proc. Natl. Acad. Sci USA 88, 189-93(1991)); and Sandwich hybridization method (e.g., U.S. Pat. No. 5,288,609, which is incorporated herein by reference in their entireties).

In a ninth aspect of this disclosure, there is provided application of a viscous biological sample liquefying composition as described in the first aspect of this disclosure, the liquefying agent as described in the second aspect of this disclosure, the viscous biological sample processing combination product as described in the third aspect of this disclosure, the viscous biological sample liquefying agent as described in the fourth aspect of this disclosure, and the combination product for liquefying and preserving a viscous biological sample as described in the fifth aspect of this disclosure or the kit as described in the sixth aspect of this disclosure.

In some embodiments of this disclosure, this disclosure as described is for non-diagnostic and therapeutic purposes.

In some embodiments of this disclosure, this disclosure as described is for diagnostic or therapeutic purposes. In some embodiments of this disclosure, there is provided an application of the viscous biological sample liquefying agent as described in the fourth aspect of this disclosure, or a combination product (as described in the fifth aspect of this disclosure), or a liquefaction kit (as described in the sixth aspect of this disclosure) thereof in the nucleic acid direct amplification or direct detection. In some embodiments therein, the application as described is for non-diagnostic and therapeutic purposes. In some embodiments therein, the combination product includes the viscous biological sample liquefying agent as described in the fourth aspect of this disclosure and an optional preservation component. In some embodiments therein, the liquefaction kit includes the viscous biological sample liquefying agent as described in any of the fourth aspects of this disclosure. In some embodiments, the application refers to the use in conjunction with a real-time quantitative fluorescent PCR instrument or with a portable nucleic acid detector.

In some embodiments of this disclosure, there is provided an application of the combination product for liquefying and preserving a viscous biological sample as described in the third aspect of this disclosure and the kit thereof in the nucleic acid direct amplification or direct detection.

In some embodiments of this disclosure, there is provided an application of the combination product for liquefying and releasing a viscous biological sample as described in the third aspect of this disclosure and the kit thereof in the nucleic acid direct amplification or direct detection.

In some embodiments of this disclosure, there is provided an application of the viscous biological sample liquefying agent as described in the fourth aspect of this disclosure, or the combination product for liquefying and preserving a viscous biological sample as described in the fifth aspect of this disclosure, or the liquefaction kit as described in the sixth aspect of this disclosure in the nucleic acid direct amplification or direct detection.

In some embodiments of this disclosure, the viscous biological sample liquefying agent as described in the fourth aspect of this disclosure can be flexibly combined with different nucleic acid detection platforms, including but not limited to real-time quantitative fluorescent PCR instrument or portable nucleic acid detector (e.g., iPonatic mobile molecular detection system). Wherein, iPonatic mobile molecular detection system is a nucleic acid detection system that has been publicly launched by SANSURE BIOTECH INC., and real-time quantitative fluorescent PCR instrument is a common instrument familiar to those skilled in the art.

After liquefying with the viscous biological sample liquefying agent in the fourth aspect of this disclosure, the nucleic acid can be directly amplified and detected, while the traditional liquefying agent needs to be liquefied first, then extracted, and then the amplification and detection can be carried out. In contrast, using the viscous biological sample liquefying agent of this disclosure can shorten the whole detection time (from liquefaction to completion of nucleic acid detection) to less than 40 min, and can improve the detection efficiency by more than 4 times.

The embodiments of this disclosure will be described in detail below in combination with examples. It should be understood that these examples are only used to describe the present disclosure rather than limiting the scope of the present disclosure. The experimental methods without indicating specific conditions in the following examples will preferentially make reference to the guidance given in this disclosure, and it can further refer to the experimental manual or conventional conditions in the field, and other experimental methods known in the field, or according to the conditions recommended by the manufacturer.

In the following examples, the measurement parameters of raw material components may have slight deviation from the weighing accuracy range, unless otherwise specified. When referring to temperature and time parameters, it allows the acceptable deviations caused by instrument test accuracy, operation accuracy or a combination of the two.

DTT, dithiothreitol has a concentration in mass volume ratio.

SDS, sodium dodecyl sulfate.

The nucleic acid extraction or purification reagent (One-step RNA releasing agent) produced by SANSURE BIOTECH INC., is used in the following examples 1-13, Specification and Model: Specification 24T, Model 51014.

The six respiratory pathogen nucleic acid assay kits (PCR fluorescent probe assay) with Batch 2021001, and SFDA Certified No.: 20213400256 are used in the following examples 1-14.

### Example 1: Test of Liquefaction Effect

In this example, guaifenesin, sodium hydroxide and zirconia beads were used to prepare the liquefying reagent of the experimental to final concentration of 100 mM of guaifenesin, 10 mM of sodium hydroxide, and 1 g/mL of zirconia beads (the diameter was 1 mm), and purified water was used as a solvent for reagent preparation, which was denoted as the experimental group.

4 sputum samples (I, II, III, and IV) that were viscous visually, were collected, each one of these sputum samples was fully mixed separately, and 6 aliquots (2 mL each) were taken from each sample and marked as Nos. 1-6, for ready use.
6 mL of reagent of the experimental group was added to sample No. 1;
6 mL of dithiothreitol solution ((2% (w/v) DTT) was added to sample No. 2;
6 mL of sodium hydroxide solution (0.1 M) was added to sample No. 3;
6 mL of reagent prepared to a final concentration of 100 mM of acetylcysteine, 10 mM of sodium hydroxide and 1 g/mL of zirconia beads (diameter: 1 mm) was added to sample No. 4;
6 mL of guaifenesin with a final concentration of 100 mM was added to sample No. 5;
10 mM of sodium hydroxide was added to sample No. 6, which was then shaken and mixed well at room temperature for 5 minutes, then was used to process the sputum. The pH was adjusted to neutral, and then 100 mM of guaifenesin was added, which was then shaken and mixed well at room temperature for 5 minutes.
The above samples were shaken and mixed well at room temperature for 5 minutes;
Four liquefied samples obtained in the previous steps were aspirated with a pipette, the aspiration process was observed and recorded. The results are shown in Table 1.

The results show that the sputum liquefied by this method can be successfully aspirated by a 10 μL pipette, it is proven that this method has a good liquefaction effect on sputum, and the viscosity of the sample is significantly reduced.

It was found that replacing guaifenesin in this method system with acetylcysteine (sample No. 4), the liquefaction effect decreased. In addition, the liquefaction effect further decreased in the case of a single guaifenesin component (sample No. 5).

TABLE 1

| | | | | | |
|---|---|---|---|---|---|
| Sample No | Sample group | I | II | III | IV |

| | | |
|---|---|---|
| No. 1 | Experimental | The 4 samples are completely |
| | group | liquefied, aspiratable and |
| | | operatable by a 10 μL pipette |
| No. 2 | DTT method | Visible sputum, Unable to operate |
| | | because the pipette is blocked |
| No. 3 | Sodium hydroxide | Visible sputum, Unable to operate |
| | method | because the pipette is blocked |
| No. 4 | Acetylcysteine | Substantially liquefied, there |
| | | is wiredrawing phenomenon |
| | | in the tip of the pipette |
| No. 5 | Single guaifenesin | No visible sputum, Unable |
| | group | to operate because the |
| | | pipette is blocked |
| No. 6 | additional guaifenesin- | No visible sputum, Unable |
| | adding group with a | to operate because the |
| | neutral adjusted pH | pipette is blocked |
| | post sodium hydroxide | |
| | treatment | |

In addition, further, for the above single guaifenesin group (sample No. 5 which was processed under normal temperature and neutral conditions for 30 min without adding zirconia beads), the liquefaction effect was general, and there was wiredrawing phenomenon when aspirating individual thick sputum with a pipette, but no sputum was visible. For sample No. 5, the liquefaction effect can be substantially equivalent to that of the experimental group heated at 95° C. for 30 min.

### Example 2: Test of Effect of Different Concentrations of Liquefaction Components Combined with Zirconia Beads

In this example, the liquefying reagent of the experimental group was consisted of one or more of the following components: guaifenesin, sodium hydroxide and zirconia beads.
Experimental group 1: the final concentration was 100 mM of guaifenesin, 10 mM of sodium hydroxide, 1 g/mL of zirconia beads (the diameter was 1 mm), and the solvent used to prepare the reagent was purified water.
Experimental group 2: the final concentration was 1 mM of guaifenesin, 1000 mM of sodium hydroxide, 1 g/mL of zirconia beads (the diameter was 1 mm), and the solvent used to prepare the reagent was purified water.
Experimental group 3: the final concentration was 1 mM of guaifenesin, 1 g/mL of zirconia beads (the diameter was 1 mm), 0.1 mM of sodium hydroxide, and the solvent used to prepare the reagent was purified water.

4 sputum samples (1, 11, 111, and IV) that were viscous visually, were collected, each one of these sputum samples was fully mixed separately, and 3 aliquots (each 2 ml) were taken from each sample and marked as Nos. 1-3, for ready use.
6 mL of reagent of the experimental group 1 was added to sample No. 1 (pH=12 after mixing);
6 mL of reagent of the experimental group 2 was added to sample No. 2 (pH=14 after mixing);
6 mL of reagent of the experimental group 3 was added to sample No. 3 (pH=10 after mixing);
The above samples were shaken and mixed well at room temperature for 5 minutes;
Four liquefied samples obtained in the previous steps were aspirated with a pipette, the aspiration process was observed and recorded. The results are shown in Table 2.

The results show that the sputum liquefied by methods of each experimental group can be successfully aspirated by a 10 μL pipette, it is proven that the present method has a good liquefaction effect on sputum under a wide range of guaifenesin concentration and pH, and the viscosity of the sample is significantly reduced.

TABLE 2

| | | | | |
|---|---|---|---|---|
| Sample No | I | II | III | IV |

| | |
|---|---|
| No. 1 | The 4 samples were completely liquefied, |
| | aspiratable and operatable by a 10 μL pipette |
| No. 2 | The 4 samples were completely liquefied, |
| | aspiratable and operatable by a 10 μL pipette |
| No. 3 | The 4 samples were completely liquefied, |
| | aspiratable and operatable by a 10 μL pipette |

### Example 3. Rapid Detection Comparison of Liquefied Sputum Samples with Other Methods

In this example, the liquefying reagent of the experimental group was prepared with guaifenesin, sodium hydroxide and zirconia beads. The final concentration was 100 mM of guaifenesin, 10 mM of sodium hydroxide, 1 g/mL of zirconia beads (the diameter was 1 mm), and the solvent used to prepare the reagent was purified water, which was used as the experimental group.

4 sputum samples (I, II, III, and IV) that were viscous visually, were collected, each one of these sputum samples was fully mixed separately, and 5 aliquots (each 2 ml) were taken from each sample and marked as No. 1-5 for ready use.

6 mL of reagent of the experimental group was added to sample No. 1; 2.5 g of guanidine hydrochloride+0.5 g of acetylcysteine+2 g of polypropylene particles were added to sample No. 2 (the method disclosed in Patent Document No. CN108949748A); 6 mL of dithiothreitol solution (2% DTT) was added to sample No. 3; 6 mL of trypsin solution (containing pH 7.0 buffer system) was added to sample No. 4; and 6 mL of sodium hydroxide solution (0.1 M) was added to sample No. 5, all of the above samples were shaken and mixed well at room temperature for 5 minutes.

Four liquefied samples obtained in the previous steps were aspirated with a pipette (the unliquefied samples were centrifuged at 12000 rmp for 1 min and the supernatant was taken).

The nucleic acid extraction or purification reagent (one-step RNA releasing agent) produced by SANSURE BIOTECH INC., was used for extraction, and the six respiratory pathogen nucleic acid assay kit (PCR-fluorescent probe assay) produced by SANSURE BIOTECH INC., was used for detection. The detection results are shown in Table 3. Ct values of respiratory pathogen nucleic acid detection of different samples in this example are listed in Table 3; where, Ct value represents the number of cycles experienced by the fluorescence signal from each reaction tube when it reaches the set threshold. There is a linear relationship between the Ct value of each template and the logarithm of the initial copy number of the template, and the formula is as follow.

$$Ct = - 1 / lg ( 1 + Ex ) * 1 g {X}_{0} + 1 gN / lg ( 1 + Ex )$$

X₀ is the Initial template quantity, Ex is the amplification efficiency, and N is the amount of amplification product when the fluorescence amplification signal reaches the threshold intensity. n is the number of cycles of the amplification reaction. When the amplification reaches the threshold line, n=Ct.

The more the initial copy numbers, the smaller the Ct value. The standard curve can be made by using the standards with known initial copy numbers, in which the x-coordinate represents the logarithm of the initial copy number and the y-coordinate represents the Ct value. Therefore, as long as the Ct value of the unknown sample is known, the initial copy number of the sample can be calculated from the standard curve.

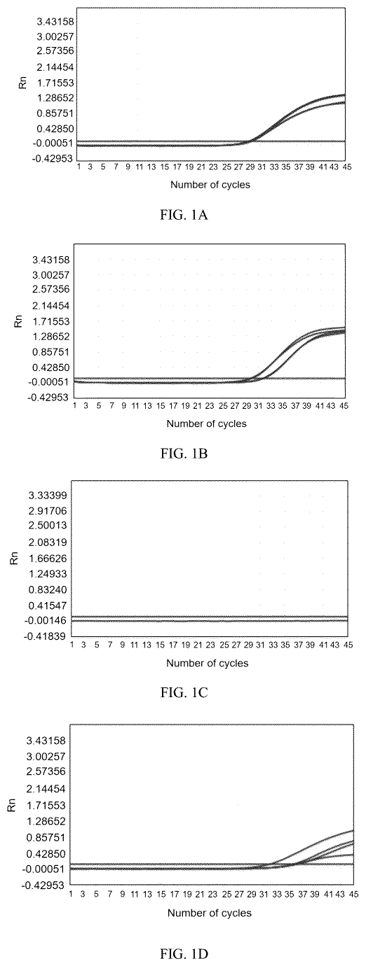

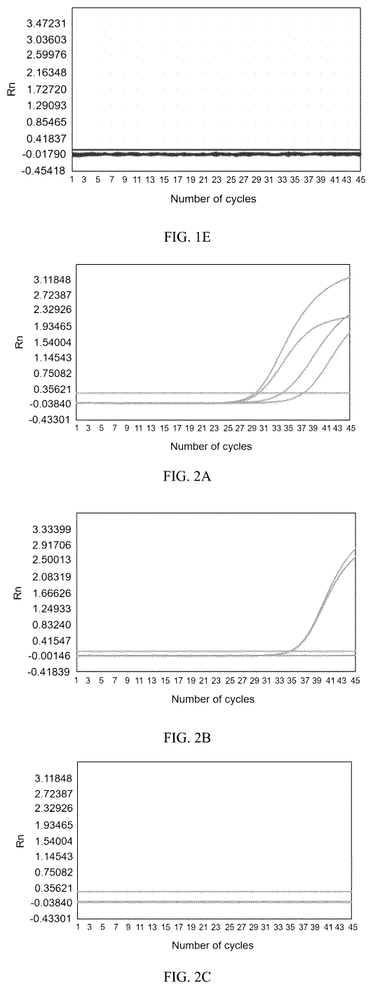

Therefore, the Ct value can reflect the concentration of the test sample. The lower the Ct value, the higher the nucleic acid concentration of the test sample, and the higher the Ct value, the lower the nucleic acid concentration of the test sample; samples with a difference of 1 Ct value showed two times difference in nucleic acid concentration, and samples with a difference of 2 Ct value showed four times difference in nucleic acid concentration, and so on. According to Table 3, human genomic DNA can be detected normally (Ct values were all less than 40) from the four liquefied samples in the experimental group of this example. Respiratory syncytial virus was positive (Ct values were less than 40), and Ct values for DNAs and RNAs detected by other methods were affected to a certain extent. It shows that samples liquefied by other methods cannot be directly rapidly extracted and detected. There is resistance to POCT (Point of Care Testing) detection system. The amplification curves of the above experimental results are shown in FIG. 1A-1E and FIG. 2A-2E, respectively. In FIG. 1A-1E, FIG. 1A represents the experimental group; FIG. 1B represents protease method; FIG. 1C represents sodium hydroxide method; FIG. 1D represents DTT method; and FIG. 1E represents acetylcysteine method. In FIG. 2A-2E, FIG. 2A represents the experimental group;

FIG. 2B represents protease method; FIG. 2C represents sodium hydroxide method; FIG. 2D represents DTT method; and FIG. 2E represents acetylcysteine method. The y-coordinate Rn in FIG. 1A-1E and FIG. 2A-2E represents the fluorescence intensity of PCR amplification products at the nth cycle. The results show that group A, group B and group D could be amplified normally, indicating that the above three groups satisfy the requirements of reagent for nucleic acid amplification detection. The highest concentration of Ct value in group A indicates that the sample liquefied in this method has the highest efficiency in detecting the target nucleic acid fragment.

TABLE 3
Ct values of respiratory pathogen nucleic acid detections in different samples in Example 3.

| | Method | | | | | |
|---|---|---|---|---|---|---|
| | Experimental group | | Sodium hydroxide method | | Protease method | |
| | Type | | | | | |
| | RNA | DNA | RNA | DNA | RNA | DNA |
| Sample | Respiratory | Human | Respiratory | Human | Respiratory | Human |
| No. | syncytial virus | genome | syncytial virus | genome | syncytial virus | genome |
| I | 29.75 | 29.63 | NoCt | NoCt | 35.38 | 31.73 |
| II | 34.10 | 25.90 | NoCt | NoCt | NoCt | 29.83 |
| III | 30.32 | 26.51 | NoCt | NoCt | 35.45 | 31.52 |
| IV | 37.51 | 24.36 | NoCt | NoCt | NoCt | 29.55 |

| | Method | | | |
|---|---|---|---|---|
| | DTT method | | Acetylcysteine method | |
| | Type | | | |
| | RNA | DNA | RNA | DNA |
| Sample | Respiratory | Human | Respiratory | Human |
| No. | syncytial virus | genome | syncytial virus | genome |
| I | NoCt | 36.17 | NoCt | NoCt |
| II | NoCt | 35.53 | NoCt | NoCt |
| III | NoCt | 36.09 | NoCt | NoCt |
| IV | NoCt | 31.93 | NoCt | NoCt |

Where: “no Ct” means that no signal value is detected, and the result is negative.

### Example 4: Verification of the Influence of the Extraction Free Amplification System (the Extracted Nucleic Acid into which the Amplification was Introduced was not Purified)

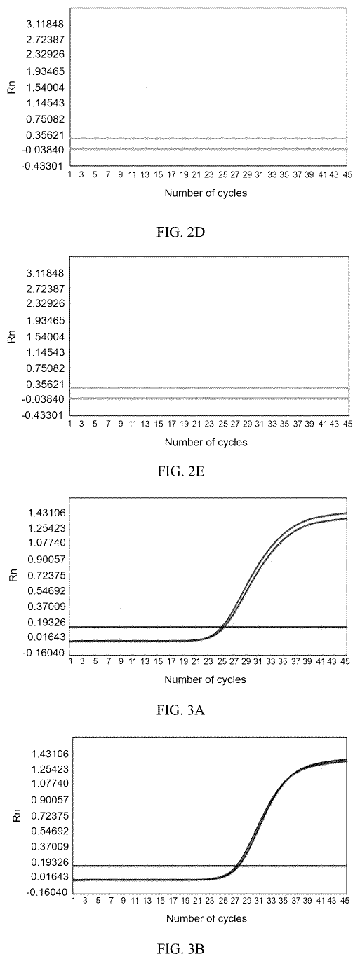

In this example, the liquefying reagent of the experimental group was prepared with guaifenesin, sodium hydroxide and zirconia beads. The final concentration was 100 mM of guaifenesin, 10 mM of sodium hydroxide, 1 g/mL of zirconia beads (the diameter was 1 mm), and the solvent used to prepare the reagent was purified water. Two throat swab samples (sample No. 1 and sample No. 2) were selected and diluted 100 limes using the liquefying agent of the experimental group of this example and normal saline (control group) as the matrix, the nucleic acid extraction or purification reagent (one-step RNA releasing agent) produced by SANSURE BIOTECH INC., was used for extraction, and the six respiratory pathogen nucleic acid assay kit (PCR-fluorescent probe assay) produced by SANSURE BIOTECH INC., was used for detection. The test results are as shown in Table 4 (Ct value) and FIG. 3A-3B. The results show that as tested by the fluorescence quantitative PCR method, the concentration value of nucleic acids in extraction free amplification system from the selected two samples diluted with the experimental group of this example is very little different from the concentration value of nucleic acids from normal saline matrix dilution, indicating that the reagent in the experimental group of this example has no inhibitory effect on the followed-up extraction of nucleic acids as well as the amplification by fluorescence quantitative PCR method and detection. Therefore, it is friendly to followed-up applications. It can be seen from FIG. 3A-3B that the nucleic acid curve shapes of the two matrix dilution tests shows normal amplification and small difference of Ct values.

TABLE 4
Ct values of respiratory pathogen nucleic acid
detections of different samples in Example 4.

| | Matrix | |
|---|---|---|
| Sample No. | Liquefying reagent of experimental group | Normal saline |
| 1 | 25.17 | 25.51 |
| 2 | 27.62 | 27.37 |

### Example 5: Liquefied Sample Stability Test

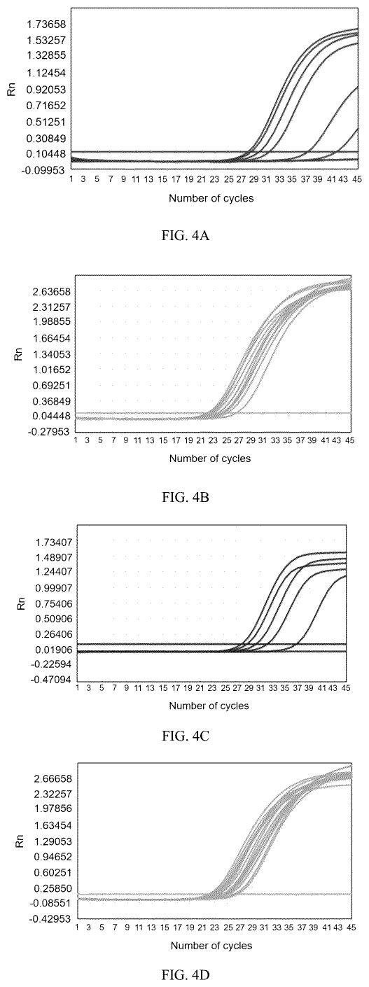

In this example, the liquefying reagent of the experimental group was prepared with guaifenesin, sodium hydroxide and zirconia beads. The final concentration was 100 mM of guaifenesin, 10 mM of sodium hydroxide, 1 g/mL of zirconia beads (the diameter was 1 mm), and the solvent used to prepare the reagent was purified water. One sputum sample that was viscous visually, was collected, and divided into two parts. One was added 3 times the volume of normal saline, ground and homogenized with a grinding mill for ready use. The other sputum sample was fully mixed with the liquefying agent in the experimental group of this example, sub-packaged into 11 samples, and marked as Nos. 1-11 for ready use. The liquefied samples stood still at 37° C. for 0 h, 4 h, 8 h, 24 h, 48 h 72 h, 96 h, 120 h, 144 h, 168 h, and 192 h, then were extracted with the nucleic acid extraction or purification reagent (one-step RNA releasing agent) produced by SANSURE BIOTECH INC., and detected with the six respiratory pathogen nucleic acid assay kit (PCR-fluorescent probe assay) produced by SANSURE BIOTECH INC. The test results are shown in Table 5 (Ct value). The results show that the RNAs from the samples liquefied with the experimental group in this example can be detected stably within 24 hours by using the rapid purification free detection system, and their concentration has no obvious change within 4 hours. The DNAs from the samples liquefied with the experimental group in this example after liquefaction can be detected stably within 192 hours by using the rapid purification free detection system, and their concentration has no obvious change within 144 hours. Its ability to detect the nucleic acids, RNA and DNA, at 37° C. is comparable to the control group. The experimental results are as shown in Table 5 and FIG. 4A-4D. It can be seen from FIG. 4A-4D that the four diagrams all show normal amplification, and the curve shape of A in FIG. 4A-4D is relatively consistent with that of C in FIG. 4A-4D. The shape of FIG. 4B is relatively consistent with that of FIG. 4C. There was little difference in the nucleic acid shelf life between the experimental group and the control group.

TABLE 5
Ct values of respiratory pathogen nucleic acid
detections of different samples in Example 5.

| | |
|---|---|
| Experimental group | Control group |

| | | | | |
|---|---|---|---|---|
| Test time | RNA | DNA | RNA | DNA |

| | | | | | |
|---|---|---|---|---|---|
| 0 | h | 28.34 | 22.31 | 27.89 | 22.31 |
| 4 | h | 28.58 | 22.05 | 28.76 | 22.79 |
| 8 | h | 29.67 | 22.62 | 30.23 | 22.69 |
| 24 | h | 31.36 | 23.07 | 32.09 | 23.02 |
| 48 | h | 37.26 | 23.71 | 36.81 | 23.68 |
| 72 | h | 41.62 | 23.68 | No Ct | 24.55 |
| 96 | h | No Ct | 24.55 | No Ct | 24.31 |
| 120 | h | No Ct | 24.31 | No Ct | 24.63 |
| 144 | h | No Ct | 24.48 | No Ct | 25.20 |
| 168 | h | No Ct | 25.18 | No Ct | 24.82 |
| 192 | h | No Ct | 26.63 | No Ct | 26.63 |

Where: “no Ct” means that no signal value is detected, and the result is negative.

### Example 6: Liquefaction Effect Test on Cervical Mucus

In this example, the liquefying reagent of the experimental group was prepared with guaifenesin sodium hydroxide and zirconia beads. The final concentration was 100 M of guaifenesin, 10 mM of sodium hydroxide, 1 g/mL of zirconia beads (the diameter was 1 mm), and the solvent used to prepare the reagent was purified water. Four cervical mucus samples that were viscous visually collected at the SANWAY CLINICAL LABORATORIES INC., were fully mixed with the liquefying agent in the experimental group of this example in a volume ratio of 1:3, and then shaken and mixed well for 5 minutes.

Four liquefied samples obtained in the previous step were aspirated with a pipette, aspiration process was observed and recorded, and the results are as shown in Table 6. The results show that the cervical mucus liquefied by the present method can be successfully aspirated by a 10 μL pipette, it is proven that the present method has a good liquefaction effect on sputum, and the viscosity of the sample is significantly reduced.

TABLE 6

| Sample No. | No. 1 | No. 2 | No. 3 | No. 4 |
|---|---|---|---|---|
| Liquefying reagent of | The 4 samples were completely liquefied, | | | |
| experimental group | aspiratable and operatable by a10 μL pipette | | | |

### Example 7: Comparison and Test of the Preserving Solution of this Disclosure and the Commercially Available Preserving Solution for their Ability to Preserve Liquefied Virus Samples and Nucleic Acids

Two clinical sputum samples (sample No. 1 and sample NO. 2) containing respiratory virus were selected. Liquefaction is carried out by the method of the experimental group in example 1. After the liquefaction was completed, the preserving solution provided by the experimental group of this example and commercially available preserving solution were added respectively.

Preparation of preserving solution reagent in the experimental group of this example: the concentrations of the following components were the final concentrations after preparation, 3 mM of citric acid, 1% (w/v) of sodium chloride, 0.8% (w/v) of potassium chloride, 1M of trehalose, 2.5% (w/v) of mannitol, 1.5% (w/v) of urea, 6% (v/v) of glycerol, 1M of glycine, 1M of isoleucine, with the solvent being sterile purified water.

The above preserving solution with ⅓ times the volume of the sample which has been subjected to completed liquefaction was added to the sample, and fully mixed.

The commercially available preserving solution (the main active ingredients included RNasin, guanidinium thiocyanate, and sodium chloride, which were greatly different from the preserving solution of the experimental group of this example): was operated according to the manufacturer's instruction.

The samples preserved in the two preserving solutions stood still at 37° C. for 0 h, 4 h, 8 h, 24 h, 48 h, 72 h, 96 h 144 h, 192 h, and 288 h, respectively synchronously (after being processed at 37° C., the samples were stored at −20° C. and thawed after all testing groups were processed for standby), then extracted with the nucleic acid extraction or purification reagent (one-step RNA releasing agent) produced by SANSURE BIOTECH INC., and detected with the six respiratory pathogen nucleic acid assay kit (PCR-fluorescent probe assay) produced by SANSURE BIOTECH INC.

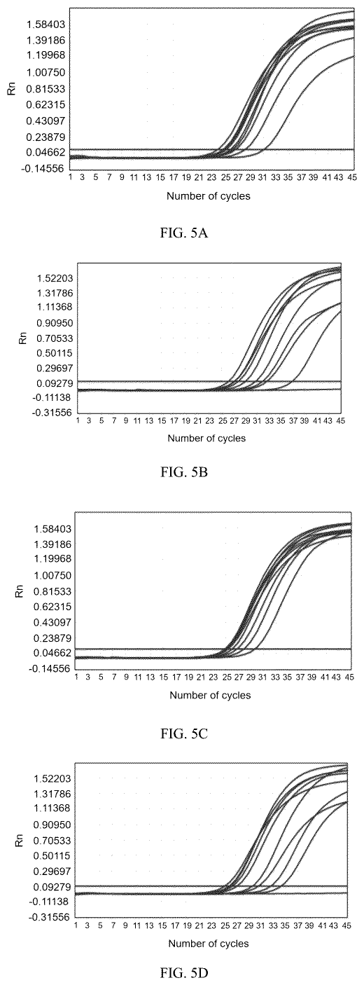

The test results are shown in Table 7 (Ct value) and FIG. 5A-5D. The results show that after the sputum samples liquefied by the above liquefaction method are preserved by the preserving solution of the experimental group in this example, the virus nucleic acids stored can be detected over a longer time than that in the commercially available preserving solution, and the detected concentration is higher than that in the commercially available preserving solution. From the concentration change of nucleic acids during 0 h-288 h, it can be found that the nucleic acids degrade significantly lower in the preserving solution of the experimental group of this example than that in the commercially available preserving solution, indicating that the preserving solution of the experimental group of this example has great advantages in detection of the preserved virus samples and nucleic acids by using fluorescent PCR, with better stability and longer durability.

TABLE 7
Ct values of respiratory virus nucleic acid
detection results of different samples in Example 7
Respiratory virus nucleic acid detection results

| | | Sample No. 1 | | Sample No. 2 | |
|---|---|---|---|---|---|
| | | Preserving | Commercially | Preserving | Commercially |
| | | solution of | available | solution of | available |
| | | experimental | preserving | experimental | preserving |
| Time | | group | solution | group | solution |
| 0 | h | 24.52 | 26.19 | 24.92 | 25.17 |
| 4 | h | 24.73 | 27.17 | 25.03 | 25.90 |
| 8 | h | 24.85 | 27.21 | 25.05 | 26.07 |
| 24 | h | 24.87 | 27.82 | 25.12 | 26.37 |
| 48 | h | 25.21 | 29.04 | 25.33 | 27.15 |
| 72 | h | 25.90 | 30.69 | 25.35 | 29.64 |
| 96 | h | 26.23 | 31.88 | 26.06 | 30.59 |
| 144 | h | 26.64 | 32.57 | 26.82 | 32.58 |
| 192 | h | 28.14 | 36.81 | 27.94 | 34.26 |
| 288 | h | 31.08 | No Ct | 29.64 | No Ct |

Where: “no Ct” means that no signal value is detected, and the result is negative.

### Example 8: Comparison and Test of the Preserving Solution of this Disclosure and the Commercially Available Preserving Solution for their Ability to Preserve Pathogen Samples and Nucleic Acids

Two clinical sputum samples (sample No. 1 and sample NO. 2) containing Neisseria gonorrhoeae were selected. Liquefaction is carried out by the method of the experimental group in example 1. After the liquefaction was completed, the preserving solution provided by the experimental group of this example and commercially available preserving solution were added respectively.

Preparation of preserving solution reagent in the experimental group of this example: the concentrations of the following components were the final concentrations after preparation, 3 mM of citric acid, 1% (w/v) of sodium chloride, 0.8% (w/v) of potassium chloride, 1 M of trehalose, 2.5% (w/v) of mannitol, 1.5% (w/v) of urea, 6% (v/v) of glycerol, 1 M of glycine, 1 M of isoleucine, with the solvent being sterile purified water.

The above preserving solution with ⅓ times the volume of the sample which has been subject to completed liquefaction was added to the sample, and fully mixed.

The commercially available preserving solution (the main active ingredients included RNasin, guanidinium thiocyanate, and sodium chloride: was operated according to the manufacturer's instructions.

The samples preserved in the two preserving solutions stood still at 37° C. for 0 h, 4 h, 8 h, 24 h, 48 h 72 h, 96 h, 144 h, 192 h, and 288 h, respectively synchronously (after being processed at 37° C., the samples were stored at −20° C. and thawed after all testing groups were processed for standby), then were extracted with the nucleic acid extraction or purification reagent (one-step RNA releasing agent) produced by SANSURE BIOTECH INC., and detected with the Neisseria gonorrhoeae nucleic acid assay kit (PCR-fluorescent probe assay) produced by SANSURE BIOTECH INC.

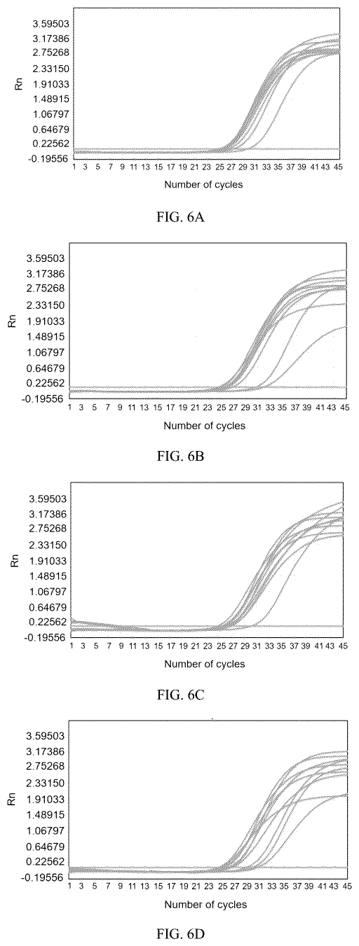

The test results are shown in Table 8 (Ct value) and FIG. 6A-6D. The results show that after the sputum samples liquefied by the above liquefaction method are preserved by the preserving solution of the experimental group in this example, the pathogen nucleic acids can be detected over a lime substantially equivalent to that in the commercially available preserving solution, and the detected concentration is higher than that in the commercially available preserving solution. From the concentration change of nucleic acids during 0 h-288 h, it can be found that the nucleic acids degrade significantly lower in the preserving solution of the experimental group of this example than that in the commercially available preserving solution, indicating that the preserving solution of the experimental group of this example has great advantages in detection of the preserved virus samples and nucleic acids by using fluorescent PCR.

TABLE 8
Ct values of Neisseria gonorrhoeae DNA nucleic acid
detection results of different samples in Example 8
Detection results of DNA nucleic acids of Neisseria gonorrhoeae

| | | Sample No. 1 | | Sample No. 2 | |
|---|---|---|---|---|---|
| | | Preserving | Commercially | Preserving | Commercially |
| | | solution of | available | solution of | available |
| | | experimental | preserving | experimental | preserving |
| Time | | group | solution | group | solution |
| 0 | h | 25.17 | 24.73 | 24.77 | 24.92 |
| 4 | h | 25.19 | 25.01 | 25.39 | 25.24 |
| 8 | h | 25.54 | 25.49 | 25.63 | 25.52 |
| 24 | h | 25.54 | 25.86 | 25.70 | 25.76 |
| 48 | h | 25.66 | 25.90 | 25.82 | 26.38 |
| 72 | h | 25.78 | 26.02 | 26.26 | 26.69 |
| 96 | h | 25.99 | 26.08 | 26.56 | 27.03 |
| 144 | h | 26.56 | 26.94 | 26.87 | 29.29 |
| 192 | h | 27.48 | 30.60 | 26.93 | 30.28 |
| 288 | h | 29.87 | 31.93 | 30.17 | 31.01 |

### Example 9: Comparison and Test of the Preserving Solution of this Disclosure and the Commercially Available Preserving Solution for their Ability to Preserve Liquefied Cell Samples and Nucleic Acids

Two clinical sputum samples (sample No. 1 and sample NO. 2) containing human exfoliated cells were selected. Liquefaction is carried out by the method of the experimental group in example 1. After the liquefaction was completed, the preserving solution provided by the experimental group of this example and commercially available preserving solution were added respectively.

Preparation of preserving solution reagent in the experimental group of this example: the concentrations of the following components were the final concentrations after preparation, 3 mM of citric acid, 1% (w/v) of sodium chloride, 0.8% (w/v) of potassium chloride, 1 M of trehalose, 2.5% (w/v) of mannitol, 1.5% (w/v) of urea, 6% (v/v) of glycerol, 1 M of glycine, 1 M of isoleucine, with the solvent being sterile purified water.

The above preserving solution with ⅓ times the volume of the sample which has been subject to completed liquefaction was added to the sample and fully mixed.

The commercially available preserving solution (the main active ingredients included RNasin, guanidinium thiocyanate, and sodium chloride: was operated according to the manufacturer's instructions.

The samples preserved in the two preserving solutions stood still at 37° C. for 0 h, 4 h, 8 h, 24 h, 48 h 72 h, 96 h, 144 h, 192 h, and 288 h, respectively synchronously (after being processed at 37° C., the samples were stored at −20° C. and thawed after all testing groups were processed for standby), then were extracted with the nucleic acid extraction or purification reagent (one-step RNA releasing agent) produced by SANSURE BIOTECH INC., and detected with the six respiratory pathogen nucleic acid assay kit (PCR-fluorescent probe assay) produced by SANSURE BIOTECH INC.

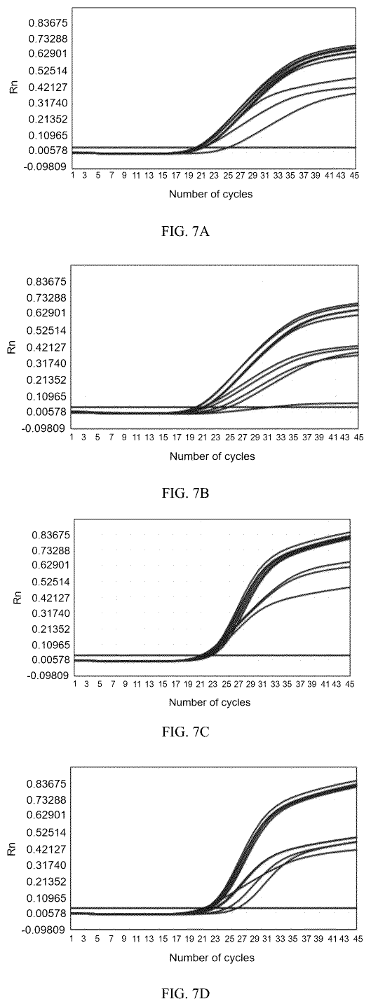

The test results are shown in Table 9 (Ct value) and FIG. 7A-7D. The results show that after the sputum samples liquefied by the above liquefaction method are preserved by the preserving solution of the experimental group in this example, the human genome nucleic acids can be detected over a time equivalent to that in the commercially available preserving solution, and the detected concentration is higher than that in the commercially available preserving solution. From the concentration change of nucleic acids during 0 h-288 h, it can be found that the nucleic acids degrade significantly lower in the preserving solution of this disclosure than that in the commercially available preserving solution, indicating that the preserving solution of this disclosure has a certain advantage in detection of the preserved virus samples and nucleic acids by using fluorescent PCR.

TABLE 9
Ct values of Human Genome DNA nucleic acid
detection results of different samples in Example 9
Human genome DNA nucleic acid detection results

| | | Sample No. 1 | | Sample No. 2 | |
|---|---|---|---|---|---|
| | | Preserving | Commercially | Preserving | Commercially |
| | | solution of | available | solution of | available |
| | | experimental | preserving | experimental | preserving |
| Time | | group | solution | group | solution |
| 0 | h | 18.00 | 18.38 | 18.96 | 19.14 |
| 4 | h | 18.14 | 18.52 | 19.02 | 19.34 |
| 8 | h | 18.37 | 19.34 | 19.34 | 19.51 |
| 24 | h | 18.39 | 19.41 | 19.51 | 19.84 |
| 48 | h | 18.96 | 19.46 | 19.63 | 20.01 |
| 72 | h | 18.97 | 19.47 | 19.84 | 20.13 |
| 96 | h | 19.02 | 20.26 | 20.01 | 20.55 |
| 144 | h | 19.05 | 21.77 | 20.13 | 20.79 |
| 192 | h | 19.08 | 23.04 | 20.42 | 22.36 |
| 288 | h | 22.47 | 30.50 | 20.43 | 23.74 |

### Example 10: Comparison and Test of Nucleic Acid Releasing Agent of this Disclosure and Commercially Available Nucleic Acid Releasing Agent for their Ability to Release Nucleic Acid from Liquefied Sputum Samples 10.1. Formulation 10-1

Preparation of releasing agent reagent in the experimental group of this example: the concentrations of the following components were the final concentration after preparation, 1% (v/v) of Tween 20 by volume percentage, 1.5% (v/v) of Triton X-100 by volume percentage, 1.5% (v/v) of ethyl phenyl polyethylene glycol by volume percentage, 10 mg/mL (250 mM) of strong base (specifically sodium hydroxide) by mass concentration, 5% (w/v) of Chelex resin by mass volume percentage, and the solvent being sterile purified water.

Commercially available nucleic acid releasing agent 1: the main components included guanidinium isothiocyanate, sodium dodecyl sulfate (SDS), and ethanol

Commercially available nucleic acid releasing agent 2: the main components included sodium hydroxide, potassium chloride, isoamyl alcohol, and Tween.

Two clinical sputum samples (sample No. 1 and sample NO. 2) containing respiratory virus, human cells, and Neisseria gonorrhoeae were selected. Liquefaction was carried out by the method of the experimental group in example 1. After the sample was completely liquefied, the nucleic acid releasing agent of the experimental group of this example and the commercially available nucleic acid releasing agent were added respectively for cleavage processing as required. After the samples were processed completely, the six respiratory pathogen nucleic acid assay kit (PCR-fluorescent probe assay) and Neisseria gonorrhoeae nucleic acid assay kit (PCR-fluorescent probe assay) produced by SANSURE BIOTECH INC., were used for detection.

Nucleic acid releasing agent in the experimental group of this example: 10 μL of samples after completed liquefaction was taken and mixed well with 10 μL of nucleic acid releasing agent, and stood still at room temperature for 10 min. Nucleic acids were reserved for ready use.

Commercially available nucleic acid releasing agent 1: operation was carried out according to the instructions (15 μL of sample and 5 μL of nucleic acid releasing agent were taken, repeatedly pipetted with a pipette to mix well, and stood still at room temperature for 10 mm). Nucleic acids were reserved for ready use.

Commercially available nucleic acid releasing agent 2: operating according to the instructions (25 μL of sample and 25 μL of nucleic acid releasing agent were taken, repeatedly blown and beaten with a pipette to mix well, and stood still at room temperature for 10 min). Nucleic acids were reserved for ready use.

Each group contained two samples, No. 1 and No. 2.

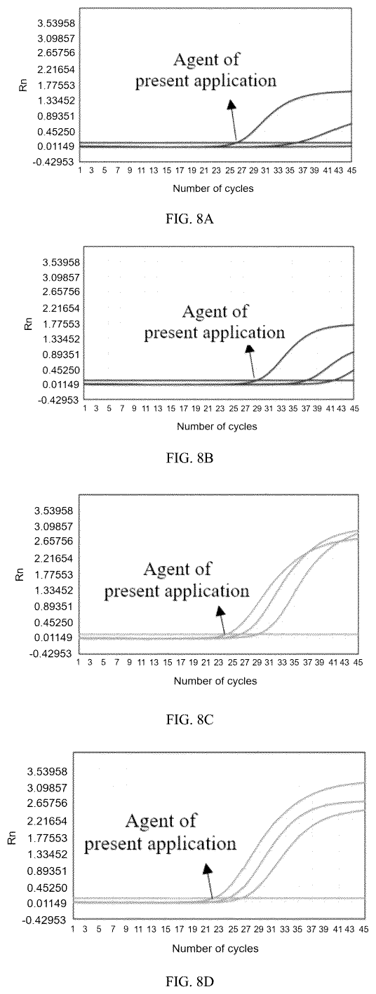

The test results (Ct value) are shown in below Tables 10-12 and FIG. 8A-8F, FIG. 8A is the RNA amplification curve of respiratory virus No. 1; FIG. 8B is the RNA amplification curve of respiratory virus No. 2; FIG. 8C is the DNA amplification curve of human cells of sample No. 1; FIG. 8D is the DNA amplification curve of human cells of sample No. 2; FIG. 8E is the DNA amplification curve of Neisseria gonorrhoeae of sample No. 1; FIG. 8F is the DNA amplification curve of Neisseria gonorrhoeae of sample No. 2; and in FIG. 8A-8F, the Ct position corresponding to the reagent of this disclosure is further marked (the intersection position of horizontal line and amplification curve in the figure); In Table 10-12, the Ct value of the releasing agent of this disclosure is at least 1 lower than that of the commercially available releasing agent, that is, the sensitivity is significantly improved. In particular, in Table 10, when detecting respiratory viruses, the Ct value is 8.13 and 9.74 lower than that in the commercially available reagents. The sample concentration is reflected from the Ct value, indicating that the reagent of this disclosure has significantly improved efficiency of nucleic acid release of samples compared with commercially available reagents. The results show that the detected concentration of viral nucleic acids released with nucleic acid releasing agent of the experimental group of this example is higher than that with commercially available releasing agent. It shows that the nucleic acid releasing agent provided in this example has greater advantages in the detection of the nucleic acids of RNA virus samples by using fluorescent PCR. The detected concentration of DNA nucleic acids released with nucleic acid releasing agent in the experimental group of this example is higher than that with commercially available releasing agent. It shows that the releasing agent of the experimental group in this example further has a certain advantage in detection of the nucleic acids of DNA samples by using fluorescent PCR.

TABLE 10
Respiratory virus

| | | Commercially | Commercially |
|---|---|---|---|
| | Releasing agent reagent | available | available |
| Sample | of the experimental | releasing | releasing |
| No. | group of this example | agent 1 | agent 2 |
| 1 | 26.35 | 36.09 | No Ct |
| 2 | 19.13 | 37.26 | 41.62 |

TABLE 11
DNA from Human cells

| | | Commercially | Commercially |
|---|---|---|---|
| | Releasing agent reagent | available | available |
| Sample | of the experimental | releasing | releasing |
| No. | group of this example | agent 1 | agent 2 |
| 1 | 24.20 | 26.37 | 29.16 |
| 2 | 22.14 | 24.34 | 26.27 |

TABLE 12
DNA from Neisseria gonorrhoeae

| | | Commercially | Commercially |
|---|---|---|---|
| | Releasing agent reagent | available | available |
| Sample | in the experimental | releasing | releasing |
| No. | group of this example | agent 1 | agent 2 |
| 1 | 21.41 | 22.90 | 22.71 |
| 2 | 21.96 | 23.32 | 22.86 |

### 10.2. Formulation 10-2, Formulation 10-3 and Formulation 10-4

According to the releasing agent formulation of the following different component dosage, the new sputum samples containing respiratory virus, Neisseria gonorrhoeae and human cells were tested by partial methods in 10.1. The results show that after the samples are processed with the releasing agent component in this disclosure in different concentration ranges to obtain nucleic acids, higher sensitivity can be achieved when detecting the nucleic acids by fluorescent PCR.

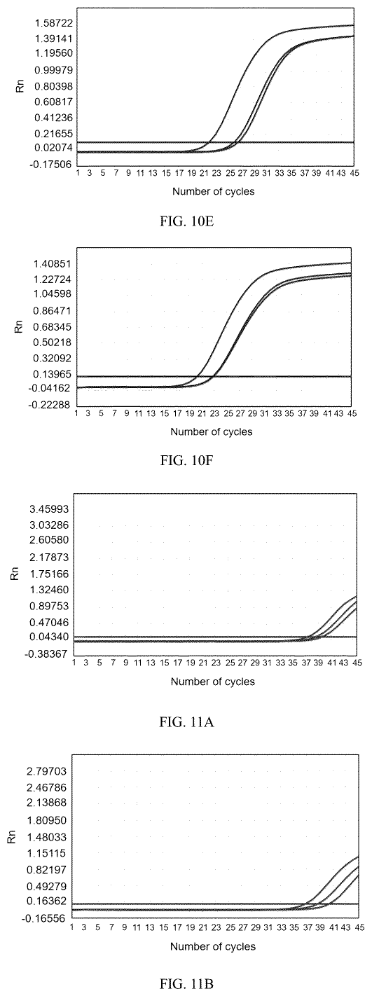

Formulation 10-2: 0.1% (v/v) of Tween 20, 1.5% (v/v) of Triton X-100, 0.1% (v/v)) of ethyl phenyl polyethylene glycol, 20 mmol/L of sodium chloride, 50 mmol/L of sodium hydroxide, 1% (w/v) of Chelex resin and sterile purified water. The results are as shown in Table 13 and FIG. 12A-12C.

TABLE 13
Test results of Formulation 10-2
Ct value concentration

| Sample | Respiratory virus | Neisseria gonorrhoeae | Human cells |
|---|---|---|---|
| 1 | 33.65 | 29.28 | 33.14 |
| 2 | 33.64 | 29.38 | 32.89 |

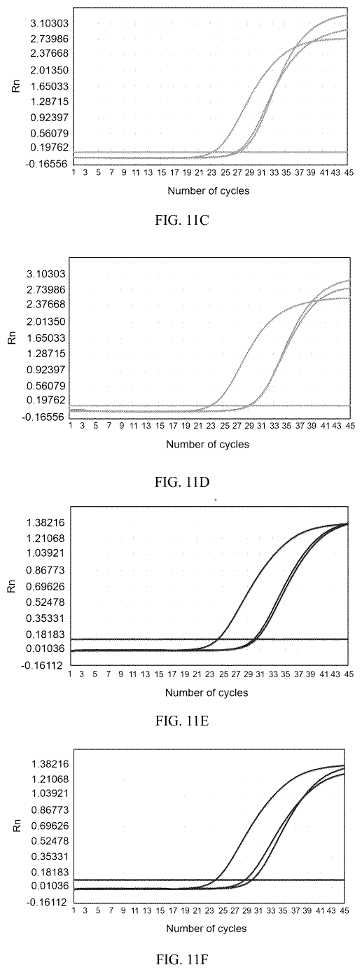

Formulation 10-3: 1% (v/v) of Tween 20, 3% (v/v) of Triton X-100, 1% (v/v)) of ethyl phenyl polyethylene glycol, 200 mmol/L of sodium chloride, 500 mmol/L of sodium hydroxide, 5% (w/v) of Chelex resin and sterile purified water. The results are as shown in Table 14 and FIG. 13A-13C.

TABLE 14
Test results of Formulation 10-3
Ct value concentration

| Sample | Respiratory virus | Neisseria gonorrhoeae | Human cells |
|---|---|---|---|
| 1 | 34.32 | 29.64 | 33.35 |
| 2 | 34.00 | 29.92 | 32.53 |

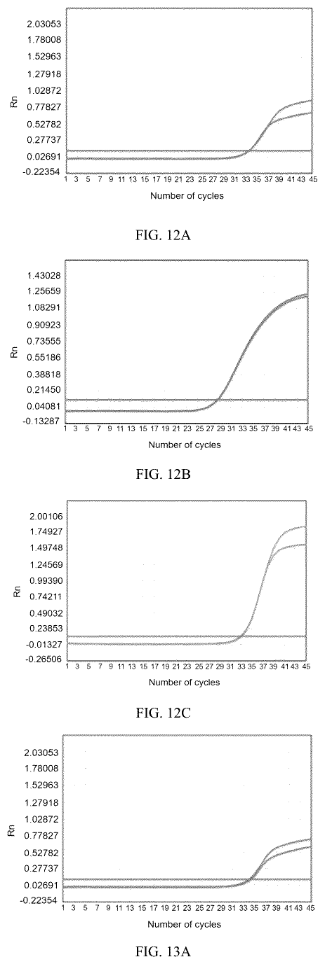

Formulation 10-4: 2% (v/v) of Tween 20, 0.1% (v/v) of Triton X-100, 3% (v/v)) of ethyl phenyl polyethylene glycol, 1000 mmol/L of sodium chloride, 1250 mmol/L of sodium hydroxide, 15% (w/v) of Chelex resin by mass volume percentage and sterile purified water. The results are as shown in Table 15 and FIG. 14A-14C.

TABLE 15
Test results of Formulation 10-4
Ct value concentration

| Sample | Respiratory virus | Neisseria gonorrhoeae | Human cells |
|---|---|---|---|
| 1 | 34.19 | 30.11 | 33.28 |
| 2 | 33.80 | 29.71 | 33.08 |

### Example 11: Comparison and Test of the Nucleic Acid Releasing Agent of this Disclosure for the Ability to Process the Sputum Samples that have been Liquefied and Added with Preserving Solution

Preparation of releasing agent reagent in the experimental group of this example: the concentrations of the following components were the final concentration after preparation, a molar concentration of 100 mmol/L of Tris-HCl, 1% (v/v) of Tween 20 by volume percentage, 1.5% (v/v) of Triton X-100 by volume percentage, 1.5% (v/v) of ethyl phenyl polyethylene glycol by volume percentage, 10 mg/mL (250 mM) of strong base (specifically sodium hydroxide) by mass concentration, 5% (w/v) of Chelex resin by mass volume percentage, and the solvent being sterile purified water.

Commercially available nucleic acid releasing agent 1 and commercially available nucleic acid releasing agent 2 were the same as those in Example 10.

Two clinical sputum samples containing respiratory virus, human cells, and Neisseria gonorrhoeae were selected. Liquefaction was carried out by the method of the experimental group in Example 7 and stored. After the sample was stored for 2 days, the nucleic acid releasing agent of the experimental group of this example and the commercially available nucleic acid releasing agent were added respectively for cleavage processing as required. After the samples were processed completely, the six respiratory pathogen nucleic acid assay kit (PCR-fluorescent probe assay) and Neisseria gonorrhoeae nucleic acid assay kit (PCR-fluorescent probe assay) produced by SANSURE BIOTECH INC., were used for detection.

Nucleic acid releasing agent in the experimental group of this example: 10 μL of samples after completed liquefaction was taken and mixed well with 10 μL of nucleic acid releasing agent, and stood still at room temperature for 10 min.

Commercially available nucleic acid releasing agent 1: operation was carried out according to the instructions (15 μL of sample and 5 μL of nucleic acid releasing agent were taken, repeatedly pipetted with a pipette to mix well, and stood still at room temperature for 10 mm). Nucleic acids were reserved for ready use.

Commercially available nucleic acid releasing agent 2: operation was carried out according to the instructions (25 μL of sample and 25 μL of nucleic acid releasing agent were taken, repeatedly pipetted with a pipette to mix well, and stood still at room temperature for 10 mm). Nucleic acids were reserved for ready use.

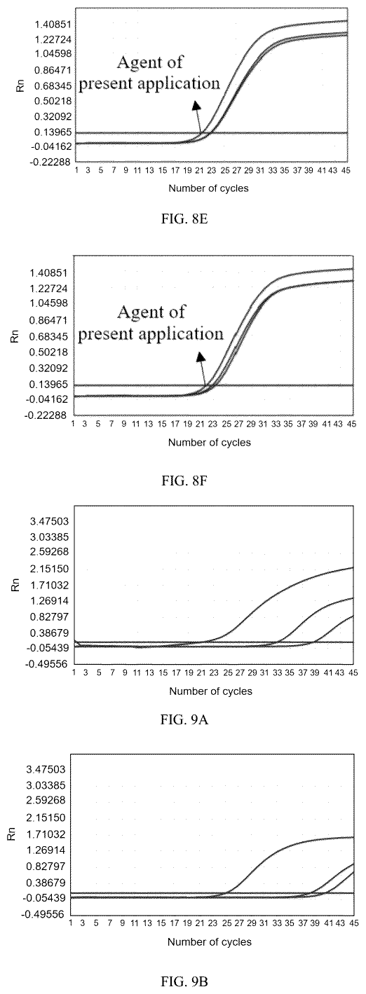

The test results (Ct value) are shown in Tables 16-18 and FIG. 9A-9F below, FIG. 9A is the RNA amplification curve of respiratory virus No. 1; FIG. 9B is the RNA amplification curve of respiratory virus No. 2; FIG. 9C is the DNA amplification curve of human cells of sample No. 1; FIG. 9D is the DNA amplification curve of human cells of sample No. 2; FIG. 9E is the DNA amplification curve of Neisseria gonorrhoeae of sample No. 1; and FIG. 9F is the DNA amplification curve of Neisseria gonorrhoeae of sample No. 2. The results show that when the RNA virus sample with the preserving solution matrix was processed with the nucleic acid releasing agent provided in this example, and then tested with the six respiratory pathogen nucleic acid assay kit (PCR-fluorescent probe assay) produced by SANSURE BIOTECH INC., the concentration of nucleic acids was higher than that processed with the commercially available releasing agent. It shows that in the case of detecting nucleic acids by using fluorescent PCR method, the releasing agent of the experimental group of this example is more efficient in processing RNA virus samples containing preserving solution matrix.

When the DNA sample containing the preserving solution matrix was processed with the nucleic acid releasing agent provided in this example, and then tested with the six respiratory pathogen nucleic acid assay kit (PCR-fluorescent probe assay) produced by SANSURE BIOTECH INC., the concentration of DNA nucleic acids was higher than that No. commercially available releasing agent. It shows that in the case of detecting nucleic acids by using fluorescent PCR method, the releasing agent in the experimental group of this example is also more efficient in processing DNA samples containing preserving solution matrix.

TABLE 16
Respiratory virus

| | | Commercially | Commercially |
|---|---|---|---|
| | Releasing agent reagent | available | available |
| Sample | of the experimental | releasing | eleasing |
| No | group of this example | agent 1 | agent 2 |
| 1 | 21.01 | 32.94 | 38.58 |
| 2 | 25.07 | 38.15 | 40.42 |

TABLE 17
DNA from Human cells

| | | Commercially | Commercially |
|---|---|---|---|
| | Releasing agent reagent | available | available |
| | of the experimental | releasing | releasing |
| Sample No | group of this example | agent 1 | agent 2 |
| 1 | 27.86 | 31.37 | 33.16 |
| 2 | 25.48 | 28.13 | 35.33 |

TABLE 18
DNA from Neisseria gonorrhoeae

| | | Commercially | Commercially |
|---|---|---|---|
| | Releasing agent reagent | available | available |
| | of the experimental | releasing | releasing |
| Sample No | group of this example | agent 1 | agent 2 |
| 1 | 24.83 | 25.60 | 26.31 |
| 2 | 24.86 | 26.08 | 26.69 |

### Example 12: Comparison and Test of the Nucleic Acid Releasing Agent of this Disclosure and Commercially Available Nucleic Acid Releasing Agent for the Ability to Release Nucleic Acid from Liquefied Sputum Samples 12.1. Formulation 12-1

Preparation of releasing agent in the experimental group of this example: the concentration of the following components were the final concentrations after preparation, 100 mmol/L of potassium chloride, 0.25 mmol/L of Surfactin, 1% of sodium dodecyl benzene sulfonate by mass volume ratio, and 0.5% (v/v) of ethanol, 500 mmol of strong base (sodium hydroxide), the solvent being sterile purified water.

Commercially available nucleic acid releasing agent 1 and commercially available nucleic acid releasing agent 2 were the same as those in Example 10.

Two clinical sputum samples (sample No. 1 and sample NO. 2) containing respiratory virus, Neisseria gonorrhoeae, and human cells were selected. Liquefaction was carried out by the method of the experimental group in example 1. After the sample was completely liquefied, the nucleic acid releasing agent of this disclosure and the commercially available nucleic acid releasing agent were added for cleavage processing as required. After the samples were processed completely, the six respiratory pathogen nucleic acid assay kits (PCR-fluorescent probe assay) and Neisseria gonorrhoeae nucleic acid assay kits (PCR-fluorescent probe assay) produced by SANSURE BIOTECH INC., were used for detection.

Nucleic acid releasing agent of the experimental group of this example: 10 μL of samples after completed liquefaction was taken and mixed well with 10 μL of nucleic acid releasing agent, and stood still at room temperature for 10 min.

Commercially available nucleic acid releasing agent 1: operation was carried out according to the instructions (15 μL of sample and 5 μL of nucleic acid releasing agent were taken, repeatedly pipetted with a pipette to mix well, and stood still at room temperature for 10 mm). Nucleic acids were reserved for ready use.

Commercially available nucleic acid releasing agent 2: operation was carried out according to the instructions (25 μL of sample and 25 μL of nucleic acid releasing agent were taken, repeatedly pipetted with a pipette to mix well, and stood still at room temperature for 10 mm). Nucleic acids were reserved for ready use.

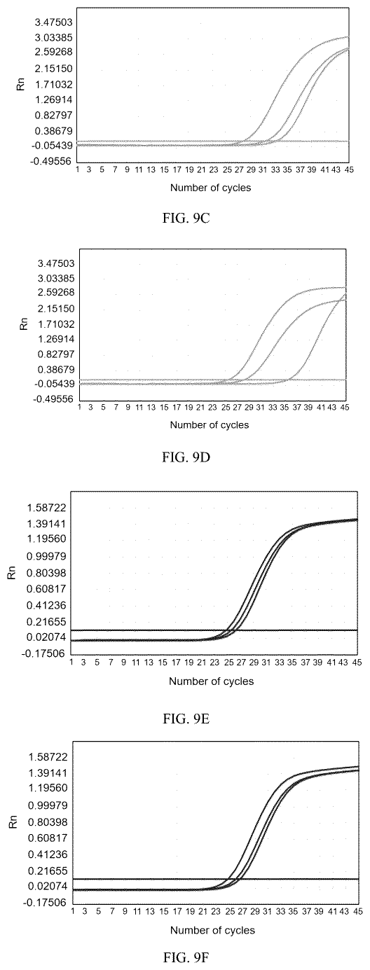

The test results (Ct value) are shown in Tables 19-21 and FIG. 10A-10F. FIG. 10 is the RNA amplification curve of respiratory virus No. 1; FIG. 10B is the RNA amplification curve of respiratory virus No. 2; FIG. 10C is the DNA amplification curve of human cells of sample No. 1; FIG. 10D is the DNA amplification curve of human cells of sample No. 2; FIG. 10E is the DNA amplification curve of Neisseria gonorrhoeae of sample No. 1; and FIG. 10F is the DNA amplification curve of Neisseria gonorrhoeae of sample No. 2. The results show that the concentration of viral nucleic acid detected with nucleic acid releasing agent of the experimental group of this example is higher than that with commercially available releasing agent. It shows that the releasing agent of this disclosure has a certain advantage in the detection of the nucleic acids of RNA virus samples by using fluorescent PCR. The concentration of DNA nucleic acid detected with nucleic acid releasing agent of the experimental group of this example is higher than that with commercially available releasing agent. It shows that the releasing agent of the experimental group of this example has a greater advantage in the detection of the nucleic acids of DNA samples by using fluorescent PCR.

TABLE 19
Respiratory virus

| | | Commercially | Commercially |
|---|---|---|---|
| | Releasing agent reagent | available | available |
| | of the experimental | releasing | releasing |
| Sample No | group of this example | agent 1 | agent 2 |
| 1 | 34.26 | 36.81 | 38.15 |
| 2 | 35.65 | 39.14 | 38.69 |

TABLE 20
DNA from Human cells

| | | Commercially | Commercially |
|---|---|---|---|
| | Releasing agent reagent | available | available |
| | of the experimental | releasing | releasing |
| Sample No | group of this example | agent 1 | agent 2 |
| 1 | 23.23 | 30.85 | 31.43 |
| 2 | 24.28 | 30.90 | 32.82 |

TABLE 20
DNA from Neisseria gonorrhoeae

| | | Commercially | Commercially |
|---|---|---|---|
| | Releasing agent reagent | available | available |
| | of the experimental | releasing | releasing |
| Sample No | group of this example | agent 1 | agent 2 |
| 1 | 21.96 | 25.95 | 26.54 |
| 2 | 20.26 | 22.77 | 22.90 |

### 12.2. Formulation 12-2, Formulation 12-3 and Formulation 12-4

According to the releasing agent formulation of the following differing component dosage, the new sputum samples containing respiratory virus, Neisseria gonorrhoeae and human cells were tested by partial methods in 12.1. The results show that when the samples are processed with the releasing agent component in this disclosure in different concentration ranges to obtain nucleic acids, higher releasing effect can be achieved when detecting the nucleic acids by fluorescent PCR.

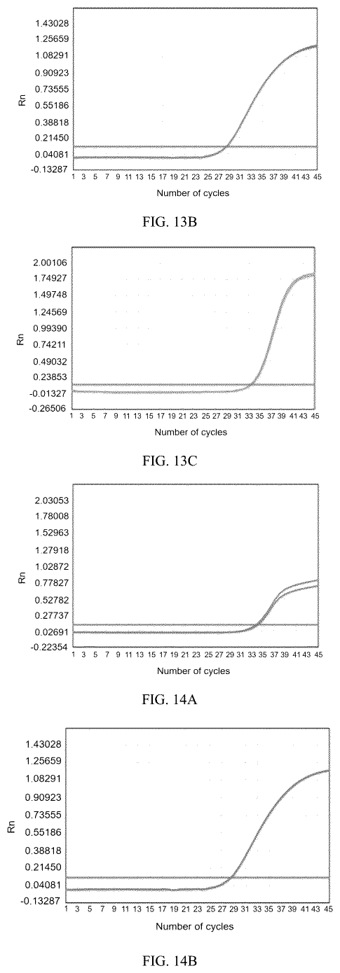

Formulation 12-2: 0.01 mmol/L of Surfactin, 0.8% (w/v) of dodecyl benzene sulfonate, 50 mmol/L of sodium chloride, 0.05% (v/v) of ethanol and 100 mmol/L of sodium hydroxide, and sterile purified water. The results are as shown in Table 22 and FIG. 15A-15C.

TABLE 22
Test results of Formulation 12-2
Ct value concentration

| Sample No | Respiratory virus | Neisseria gonorrhoeae | Human cells |
|---|---|---|---|
| 1 | No Ct | 28.57 | 33.03 |
| 2 | No Ct | 28.70 | 33.41 |

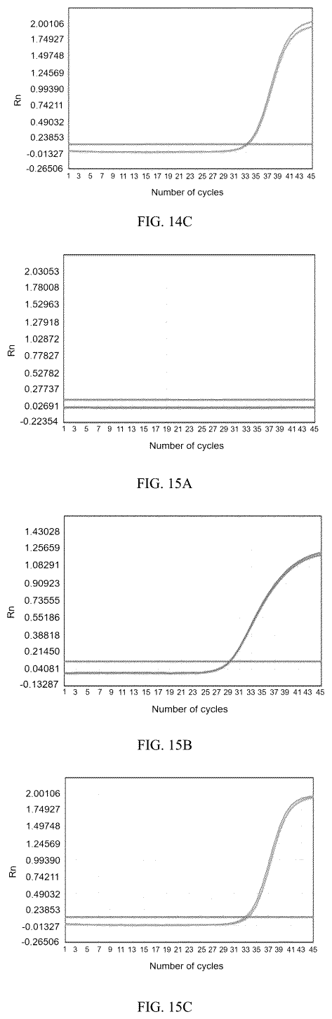

Formulation 12-3: 0.1 mmol/L of Surfactin, 2% (w/v) of dodecyl benzene sulfonate, 1200 mmol/L of sodium chloride, 0.1% (v/v) of ethanol and 500 mmol/L of sodium hydroxide, and sterile purified water. The results are as shown in Table 23 and FIG. 16A-16C.

TABLE 23
Test results of Formulation 12-3
Ct value concentration

| Sample No | Respiratory virus | Neisseria gonorrhoeae | Human cells |
|---|---|---|---|
| 1 | No Ct | 28.63 | 33.24 |
| 2 | No Ct | 28.64 | 33.50 |

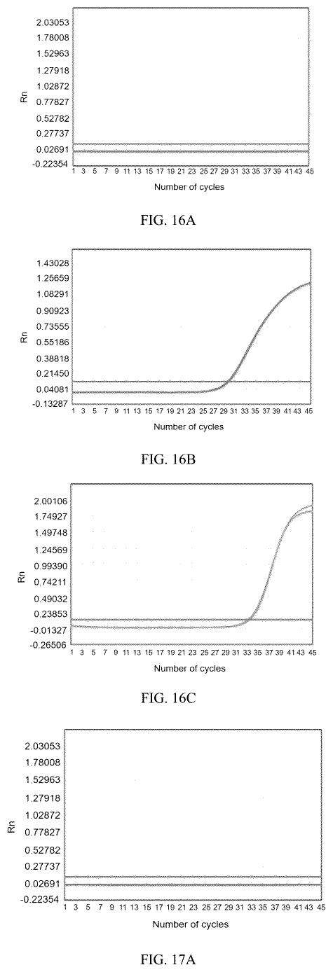

Formulation 12-4: 0.5 mmol/L of Surfactin, 0.01% (w/v) of dodecyl benzene sulfonate, 500 mmol/L of sodium chloride, 1% (v/v) of ethanol and 1250 mmol/L of sodium hydroxide, and sterile purified water. The results are as shown in Table 24 and FIG. 17A-17C.

TABLE 24
Test results of Formulation 12-4
Ct value concentration

| Sample No | Respiratory virus | Neisseria gonorrhoeae | Human cells |
|---|---|---|---|
| 1 | No Ct | 28.52 | 33.31 |
| 2 | No Ct | 28.64 | 32.67 |

### Example 13: Comparison and Test of the Nucleic Acid Releasing Agent of this Disclosure for the Ability to Process the Sputum Samples that have been Liquefied and Added with Preserving Solution

Preparation of releasing agent reagent of the experimental group of this example: the concentrations of the following components were the final concentrations after preparation. The preparation of the releasing agent reagent of this disclosure: the concentrations of the following component were the final concentrations after preparation, 100 mmol/L of potassium chloride, 0.25 mmol/L of Surfactin, 1% of lithium dodecyl benzene sulfonate by mass volume ratio, and 0.5% (v/v) of ethanol, 500 mmol of strong base (sodium hydroxide), the solvent being sterile purified water.

Two clinical sputum samples (sample No. 1 and sample NO. 2) containing respiratory virus, human cells, and Neisseria gonorrhoeae were selected. The samples were liquefied and preserved according to the method of the experimental group in Example 7. After the sample was preserved for 2 days, the nucleic acid releasing agent of the experimental group of this example and the commercially available nucleic acid releasing agent were added respectively for cleavage processing as required. After the samples were processed completely, the six respiratory pathogen nucleic acid assay kits (PCR-fluorescent probe assay) and Neisseria gonorrhoeae nucleic acid assay kits (PCR-fluorescent probe assay) produced by SANSURE BIOTECH INC., were used for detection.

Nucleic acid releasing agent in the experimental group of this example: 10 μL of samples after completed liquefaction was taken and mixed well with 10 μL of nucleic acid releasing agent, and stood still at room temperature for 10 min.

Commercially available nucleic acid releasing agent 1: operation was carried out according to the instructions (15 μL of sample and 5 μL of nucleic acid releasing agent were taken, repeatedly pipetted with a pipette to mix well, and stood still at room temperature for 10 mm). Nucleic acids were reserved for ready use.

Commercially available nucleic acid releasing agent 2: operation was carried out according to the instructions (25 μL of sample and 25 μL of nucleic acid releasing agent were taken, repeatedly pipetted with a pipette to mix well, and stood still at room temperature for 10 mm). Nucleic acids were reserved for ready use.

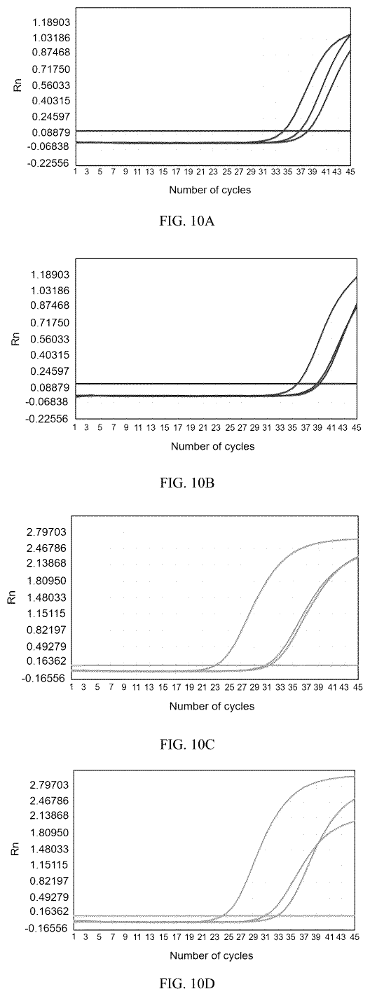

The test results (Ct value) are shown in Tables 25-27 and FIG. 11A-11F. FIG. 11A is the RNA amplification curve of respiratory virus No. 1; FIG. 11B is the RNA amplification curve of respiratory virus No. 2; FIG. 11C is the DNA amplification curve of human cells of sample No. 1; FIG. 11D is the DNA amplification curve of human cells of sample No. 2; FIG. 11E is the DNA amplification curve of Neisseria gonorrhoeae of sample No. 1; and FIG. 11F is the DNA amplification curve of Neisseria gonorrhoeae of sample No. 2. The results show that when the RNA virus sample containing the preserving solution matrix was processed with the nucleic acid releasing agent of experimental group of this example, and then tested with the six respiratory pathogen nucleic acid assay kit (PCR-fluorescent probe assay) produced by SANSURE BIOTECH INC., and the concentration of nucleic acids was higher than the processing concentration of commercially available releasing agent. It shows that in the case of detecting nucleic acids by using fluorescent PCR method, the releasing agent of the experimental group of this example is more efficient in processing RNA virus samples containing preserving solution matrix.

When the DNA sample containing the preserving solution matrix was processed with the nucleic acid releasing agent of experimental group of this example and then tested with the six respiratory pathogen nucleic acid assay kit (PCR-fluorescent probe assay) produced by SANSURE BIOTECH INC., and the concentration of DNA nucleic acids was higher than that with commercially available releasing agent. It shows that in the case of detecting the nucleic acids by using fluorescent PCR method, the releasing agent of the experimental group of this example is also more efficient in processing DNA samples containing preserving solution matrix.

TABLE 25
respiratory virus

| | | Commercially | Commercially |
|---|---|---|---|
| | Releasing agent | available | available |
| | of the experimental | releasing | releasing |
| Sample No | group of this example | agent 1 | agent2 |
| 1 | 37.38 | 38.68 | 39.59 |
| 2 | 36.81 | 38.69 | 40.12 |

TABLE 26
Human DNA

| | | Commercially | Commercially |
|---|---|---|---|
| | Releasing agent | available | available |
| | of the experimental | releasing | releasing |
| Sample No | group of this example | agent 1 | agent 2 |
| 1 | 23.28 | 27.51 | 30.37 |
| 2 | 23.10 | 29.01 | 29.99 |

TABLE 27
DNA of Neisseria gonorrhoeae

| | | Commercially | Commercially |
|---|---|---|---|
| | Releasing agent | available | available |
| | of the experimental | releasing | releasing |
| Sample No | group of this example | agent 1 | agent 2 |
| 1 | 24.31 | 30.03 | 30.37 |
| 2 | 24.03 | 29.01 | 29.99 |

The nucleic acid extraction or purification reagent (One-step RNA releasing agent) with Specification 24T and Model S1014 produced by SANSURE BIOTECH INC., was used in the following examples 14-17.

The six respiratory pathogen nucleic acid assay kit (PCR fluorescent probe assay) corresponded to SFDA Certified No. 20213400256 was used in the following examples 14-17.

The Neisseria gonorrhoeae nucleic acid assay kit with Registration No. 20153400086 and Model 48T produced by SANSURE BIOTECH INC. was used in the following examples 14-17 (PCR-fluorescent probe assay).

In the following examples, “room temperature” refers to 20° C.-30° C., unless otherwise specified.

In the liquefying agent of the viscous biological sample of this disclosure in the following examples, no other components including nuclease inhibitor (no RNase inhibitor and DNase inhibitor) were added, unless otherwise specified. The viscous biological sample liquefying agent provided in this disclosure can achieve better direct amplification effect without adding nuclease inhibitor; However, it should be understood that the viscous biological sample liquefying agent of this disclosure further allows the addition of other auxiliary components including but not limited to nuclease inhibitors (including but not limited to those as described above) to achieve further optimization purposes (such as further nucleic acid detection effect); It should be understood that the addition of nuclease inhibitor does not affect the function of the viscous biological sample liquefying agent of this disclosure. The technical solutions obtained from the foregoing changes and from similar changes thereto are also within the scope of protection of this disclosure.

### Example 14: Experimental Effect Verification of the Directly Expandable Liquefying Agent of this Disclosure 14.1. Experimental Solution

Liquefying agent in the experimental group: the concentrations of the following components were the final concentrations after preparation, 100 mmol/L of guaifenesin, 10 mmol/L of sodium hydroxide, 1 g/mL of zirconia beads (the diameter was 1 mm), the dosage of Chelex resin of 8% (w/v), 50 mmol/L of Surfactin, the solvent used to prepare reagent being purified water. The pH of the liquefying agent was about 12.5.

The liquefying agent of the control group was prepared with guaifenesin, sodium hydroxide and zirconia beads. The final concentration was 100 mM of guaifenesin, 10 mM of sodium hydroxide, and 1 g/mL of zirconia beads (the diameter was 1 mm), respectively, and the solvent used to prepare the reagent was purified water. The pH of the liquefying agent was about 12.5.

Viscous biological sample: two clinical sputum samples containing respiratory virus, human cells, and Neisseria gonorrhoeae were selected. It was divided into two parallel parts and denoted as sample No. 1 and sample No. 2, respectively.

Sample No. 1: this sample was liquefied with liquefying agent of the experimental group and liquefying agent of the control group, respectively. The liquefaction condition: the liquefaction liquid at 3 times the volume of the sample was mixed with the sample, evenly mixed, and stood still at room temperature for 10 mi. Upon being completely processed, the samples were directly used as the nucleic acid sample to be tested for direct amplification experiment, and detected with the six respiratory pathogen nucleic acid assay kits (PCR-fluorescent probe assay) and Neisseria gonorrhoeae nucleic acid assay kits (PCR-fluorescent probe assay) produced by SANSURE BIOTECH INC., according to the instructions. For the process from sampling to the end of detection i.e., from the beginning of liquefaction to the completion of nucleic acid detection, it totally takes about 36 minutes for the experimental group and about 4 hours for the control group.

Sample No. 2: this sample was liquefied with liquefying agent of the experimental group and liquefying agent of the control group, respectively. The completely liquefied samples were extracted with the nucleic acid extraction or purification reagent (One-step RNA releasing agent) produced by SANSURE BIOTECH INC., followed by the detection with the six respiratory pathogen nucleic acid assay kit (PCR-fluorescent probe assay) produced by SANSURE BIOTECH INC. and the Neisseria gonorrhoeae nucleic acid assay kit (PCR-fluorescent probe assay) produced by SANSURE BIOTECH INC. according to the instructions. For the process from sampling to the end of detection, i.e., from the beginning of liquefaction to the completion of nucleic acid detection, it totally takes about 36 minutes for the experimental group and about 4 hours for the control group. The detection efficiency was increased by more than 5 times.

### 14.2. Detection Results

The liquefaction results are as shown in Table 28. The results show that the liquefying agent of the experimental group could liquefy sputum in a short time (less than 5 min, even less than 3 min), and the liquefaction effect was good.

TABLE 28
Liquefaction results

| | | Experimental group | | Control group | |
|---|---|---|---|---|---|
| Liquefaction | | Sample | Sample | Sample | Sample |
| time | | No. 1 | No. 2 | No. 1 | No. 2 |
| 1 | min | Partial | Partial | Partial | Partial |
| | | liquefaction | liquefaction | liquefaction | liquefaction |
| 3 | min | Complete | Complete | Complete | Complete |
| | | liquefaction | liquefaction | liquefaction | liquefaction |
| 5 | min | Complete | Complete | Complete | Complete |
| | | liquefaction | liquefaction | liquefaction | liquefaction |
| 10 | min | Complete | Complete | Complete | Complete |
| | | liquefaction | liquefaction | liquefaction | liquefaction |

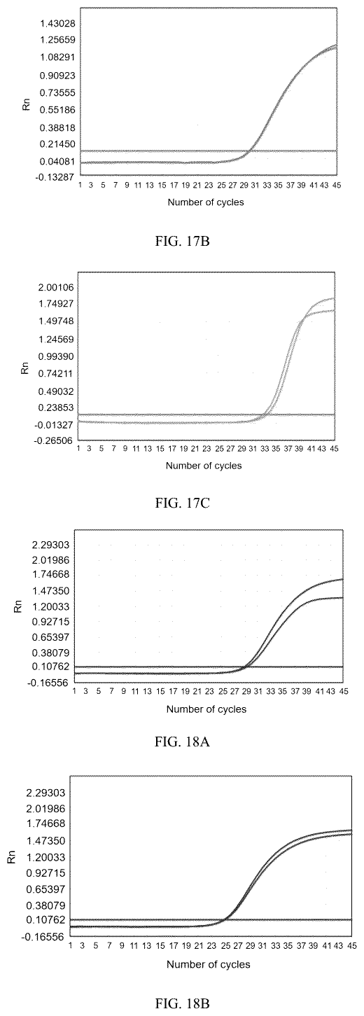

The nucleic acid amplification results are as shown in FIG. 18A-18F and Tables 29-31. FIG. 18A-18F show the amplification curve, where the y-coordinate Rn represents the fluorescence intensity of PCR amplification products at the nth cycle, and the x-coordinate represents the number of cycles. Ct value can be determined according to the amplification curve. Ct value represents the number of cycles experienced by the fluorescence signal in each reaction tube when it reaches the set threshold. There is a linear relationship between the Ct value of each template and the logarithm of the initial copy number of the template, and the formula is as follows.

$$Ct = - 1 / lg ( 1 + Ex ) * 1 g {X}_{0} + 1 gN / lg ( 1 + Ex )$$

X₀ is the Initial template quantity, Ex is the amplification efficiency, and N is the amount of amplification product when the fluorescence amplification signal reaches the threshold intensity. n is the number of cycles of the amplification reaction. When the amplification reaches the threshold line, n=Ct.

The more the initial copy numbers, the smaller the Ct value. The standard curve can be made by using the standards with known initial copy numbers, in which the x-coordinate represents the logarithm of the initial copy number and the y-coordinate represents the Ct value. Therefore, as long as the Ct value of the unknown sample is known, the initial copy number of the sample can be calculated from the standard curve.

Therefore, the Ct value can reflect the concentration of the test sample. The lower the Ct value, the higher the nucleic acid concentration of the test sample, and the higher the Ct value, the lower the nucleic acid concentration of the test sample; samples with a difference of 1 Ct value showed two times difference in nucleic acid concentration, and samples with a difference of 2 Ct value showed four times difference in nucleic acid concentration, and so on.

It can be seen from the results in FIG. 18A-18F and Tables 29-31 that the direct amplification type liquefying agent of the experimental group of this disclosure can achieve substantially the same or even better effect as the liquefying agent of the control group which is liquefied first and then released and extracted does.

TABLE 29
Ct values of detection results of
respiratory virus nucleic acids
Respiratory virus

| | Liquefying agent of | Liquefying agent |
|---|---|---|
| Sample No. | experimental group | of control group |
| 1 | 28.84 | 29.26 |
| 2 | 25.07 | 25.21 |

TABLE 30
Ct values of detection results of DNA
nucleic acids from Neisseria gonorrhoeae
DNA from Neisseria gonorrhoeae

| | Liquefying agent of | Liquefying agent |
|---|---|---|
| Sample No. | experimental group | of control group |
| 1 | 30.98 | 31.35 |
| 2 | 27.74 | 28.5 |

TABLE 31
Ct values of detection results of
DNA nucleic acids from human cells
DNA from Human cells

| | Liquefying agent of | Liquefying agent |
|---|---|---|
| Sample No. | experimental group | of control group |
| 1 | 31.18 | 30.65 |
| 2 | 26.89 | 28.01 |

14.2. The inventor further carried out experiments with three solutions, i.e., solution without adding zirconia beads (rigid microparticles), Chelex resin (adsorbing agent), or both zirconia beads and Chelex resin. The results show that the direct amplification effect could be achieved with all the solution, the Ct value of PCR amplification curve can satisfy the requirements for use, and the detection efficiency can be increased by more than 4 times.

### Example 15: Different Formulations of Viscous Biological Sample Liquefying Agents

### 15.1. Experimental Solution

Liquefying agent 1 of the experimental group: the concentrations of the following components were the final concentrations after preparation, 100 mmol/L of guaifenesin, 10 mmol/L of sodium hydroxide, 1 g/mL of zirconia beads (the diameter was 1 mm), the dosage of Chelex resin of 8% (w/v), 50 mmol/L of Surfactin, the solvent used to prepare reagent being purified water.

Liquefying agent 2 of the experimental group: the concentrations of the following components were the final concentrations after preparation, 150 mmol/L of guaifenesin, 10 mmol/L of sodium hydroxide, 1 g/mL of zirconia beads (the diameter was 1 mm), the dosage of Chelex resin of 8% (w/v), 50 mmol/L of Surfactin, the solvent used to prepare reagent being purified water.

Liquefying agent 3 of the experimental group: the concentrations of the following components were the final concentrations after preparation, 500 mmol/L of guaifenesin, 10 mmol/L of sodium hydroxide, 1 g/mL of zirconia beads (the diameter was 1 mm), the dosage of Chelex resin of 8% (w/v), 100 mmol/L of Surfactin, the solvent used to prepare reagent being purified water.

Liquefying agent 4 of the experimental group: the concentrations of the following components were the final concentrations after preparation, 20 mmol/L of guaifenesin, 10 mmol/L of sodium hydroxide, 1 g/mL of zirconia beads (the diameter was 1 mm), the dosage of Chelex resin of 8% (w/v), 200 mmol/L of Surfactin, the solvent used to prepare reagent being purified water.

Liquefying agent 5 of the experimental group: the concentrations of the following components were the final concentrations after preparation, 1000 mmol/L of guaifenesin, 10 mmol/L of sodium hydroxide, 1 g/mL of zirconia beads (the diameter was 1 mm), the dosage of Chelex resin of 8% (w/v), 1 mmol/L of Surfactin, the solvent used to prepare reagent being purified water.

Liquefying agent 6 of the experimental group: the concentrations of the following components were the final concentrations after preparation, 0 mmol/L of guaifenesin (i.e., absent), 10 mmol/L of sodium hydroxide, 1 g/mL of zirconia beads (the diameter was 1 mm), the dosage of Chelex resin of 8% (w/v), 200 mmol/L of Surfactin, the solvent used to prepare reagent being purified water.

Liquefying agent 7 of the experimental group (Comparative example 1): the concentrations of the following components were the final concentrations after preparation, 500 mmol/L of guaifenesin, 10 mmol/L of sodium hydroxide, 1 g/mL of zirconia beads (the diameter was 1 mm), the dosage of Chelex resin of 8% (w/v), 0 mmol/L of Surfactin (i.e., absent), the solvent used to prepare reagent being purified water.

Liquefying agent 8 of the experimental group: the concentrations of the following components were the final concentrations after preparation, 500 mmol/L of guaifenesin, 10 mmol/L of sodium hydroxide, 1 g/mL of zirconia beads (the diameter was 1 mm), the dosage of Chelex resin of 8% (w/v), 20 mmol/L of guanidinium isothiocyanate, the solvent used to prepare reagent being purified water.

Viscous biological sample: two clinical sputum samples (denoted as samples I and II, respectively) containing respiratory virus, human cells, and Neisseria gonorrhoeae were selected.

Liquefying agents of the above experimental groups were used for liquefaction, respectively. The liquefaction condition: the liquefying agent at 3 times the volume of the sample was mixed with the sample, evenly mixed, and stood still at room temperature for 10 min. Upon being processed completely, the samples were directly used as the nucleic acid amplification sample for direct amplification, followed by the detection with the six respiratory pathogen nucleic acid assay kit (PCR-fluorescent probe assay) and Neisseria gonorrhoeae nucleic acid assay kit (PCR-fluorescent probe assay) produced by SANSURE BIOTECH INC., according to the instructions.

### 15.2. Liquefaction Effect

The liquefaction results of the above experimental groups show that when the concentration of guaifenesin is in the range of 20 mM-1000 mM, and the process continues 5 minutes for liquefaction of sputum. The results are further shown in Table 32, wherein experimental group 7 failed to liquefy due to the absent of Surfactin.

TABLE 32

| | Liquefaction time | | | |
|---|---|---|---|---|
| | Liquefaction for 5 min | | Liquefaction for 10 min | |
| Sample No. | I | II | I | II |
| Experimental group 1 | The samples were completely liquefied, | | The samples were completely liquefied, | |
| | aspiratable and operatable by a 10 μL pipette | | aspiratable and operatable by a 10 μL pipette | |
| Experimental group 2 | The samples were completely liquefied, | | The samples were completely liquefied, | |
| | aspiratable and operatable by a 10 μL pipette | | aspiratable and operatable by a 10 μL pipette | |
| Experimental group 3 | The samples were completely liquefied, | | The samples were completely liquefied, | |
| | aspiratable and operatable by a 10 μL pipette | | aspiratable and operatable by a 10 μL pipette | |
| Experimental group 4 | The samples were completely liquefied, | | The samples were completely liquefied, | |
| | aspiratable and operatable by a 10 μL pipette | | aspiratable and operatable by a 10 μL pipette | |
| Experimental group 5 | The samples were completely liquefied, | | The samples were completely liquefied, | |
| | aspiratable and operatable by a 10 μL pipette | | aspiratable and operatable by a 10 μL pipette | |
| Experimental group 6 | The samples were completely liquefied, | | The samples were completely liquefied, | |
| | aspiratable and operatable by a 10 μL pipette | | aspiratable and operatable by a 10 μL pipette | |
| Experimental group 7 | Visible sputum, Unable to be aspirated | | Visible sputum, Unable to be aspirated | |
| (Comparative | | | | |
| example 1) | | | | |

| | | |
|---|---|---|
| Experimental group 8 | The samples were completely liquefied, | The samples were completely liquefied, |
| | aspiratable and operatable by a 10 μL pipette | aspiratable and operatable by a 10 μL pipette |

### 15.3. Direct Amplification Result

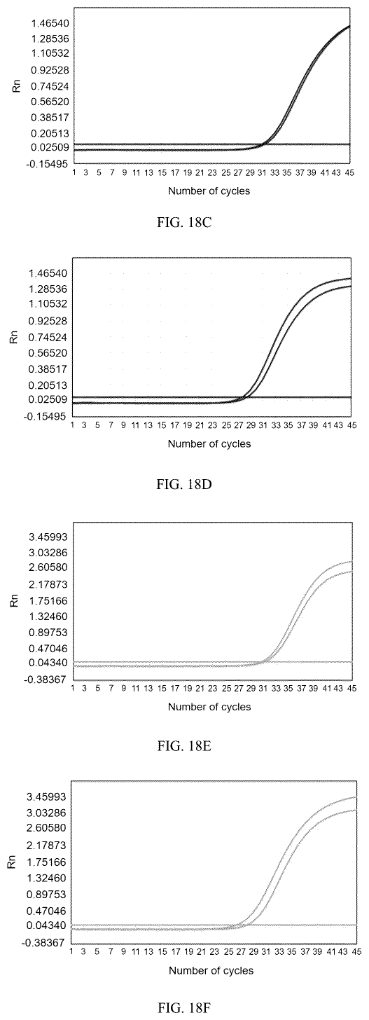

It can be found from the direct amplification test of the samples liquefied with the above liquefying agents that the experimental groups 1-5 can effectively cleave different kinds of nucleic acids, among which the experimental groups 1-3 have better effects and have little difference in the cleavage efficiency of different kinds of nucleic acids. The experimental group 6 has low nucleic acid cleavage efficiency because it could not be liquefied. Experimental group 7, of which Surfactin was absent, has low cleavage efficiency, and no signal value for respiratory virus and human cells was detected. No signal was generated in experimental group 8, indicating that Surfactin cannot be replaced. The above direct amplification results are further shown in Table 33 and FIG. 19A-19C.

TABLE 33
Ct value of amplification curve

| | Respiratory | Human | Neisseria |
|---|---|---|---|
| Type | virus | cells | gonorrhoeae |
| Experimental group 1 | 27.39 | 25.42 | 21.67 |
| Experimental group 2 | 27.28 | 25.67 | 23.10 |
| Experimental group 3 | 28.74 | 26.69 | 22.55 |
| Experimental group 4 | 29.32 | 25.60 | 23.84 |
| Experimental group 5 | 29.15 | 25.93 | 22.41 |
| Experimental group 6 | No Ct | No Ct | 23.05 |
| Experimental group 7 | No Ct | No Ct | 25.95 |
| Experimental group 8 | No Ct | No Ct | No Ct |

Where: “No Ct” means that no signal value is detected, and the result is negative.

### Example 16

Commodity amplification kit 1 (commodity amplification agent 1): SFDA Certified No.: 20173404327, 32 persons/box.

Commodity amplification kit 2 (commodity amplification agent 2): SFDA Certified No.: 20213400737, 20 persons/box.

Control amplification kit 1 (Control amplification agent): SFDA Certified No.: 20153400086, 48 persons/box. The three kits were all Neisseria gonorrhoeae nucleic acid assay kits (PCR-fluorescent probe assay).

Liquefying agent of this disclosure: the concentrations of the following components were the final concentrations after preparation, 100 mmol/L of guaifenesin, 10 mmol/L of sodium hydroxide, 1 g/mL of zirconia beads (the diameter was 1 mm), the dosage of Chelex resin of 8% (w/v), 50 mmol/L of Surfactin, the solvent used to prepare reagent being purified water.

Sputum sample: 1 clinical sputum sample containing Neisseria gonorrhoeae. The above liquefying agents were used for liquefaction. The liquefaction condition: liquefying agent at 3 times the volume of sample was mixed with the sample, mixed evenly, and stood still at room temperature for 10 mi. The liquefied sample was directly used as the nucleic acid sample for amplification and amplified and detected with Neisseria gonorrhoeae nucleic acid assay kit (PCR-fluorescent probe assay) produced by SANSURE BIOTECH INC., commodity amplification kit 1 and commodity amplification kit 2 according to the instructions.

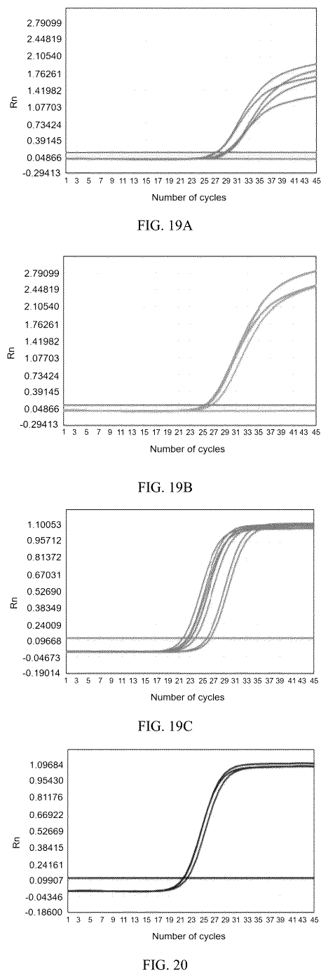

The results are as shown in Table 34 and FIG. 20. It is found that the nucleic acid obtained from the sputum sample processed with the liquefying agent in this disclosure can be amplified directly in different amplification systems. The difference between the detected concentrations is small, and the functions of rapid liquefaction, rapid nucleic acid extraction and detection of sputum samples are realized.

TABLE 34
Ct value of DNA amplification curve of Neisseria gonorrhoeae

| | Control | Commodity | Commodity |
|---|---|---|---|
| | amplification | amplification | amplification |
| Sample No. | agent | agent 1 | agent 2 |
| 1 | 21.59 | 22.32 | 21.71 |

### Example 17

### 17.1. Liquefying Agents of Different Experimental Solutions

Experimental group (viscous biological sample liquefying agent of this disclosure): the concentrations of the components were the final concentrations after preparation, 500 mmol/L of guaifenesin, 10 mmol/L of sodium hydroxide, 1 g/mL of zirconia beads (the diameter was 1 mm), the dosage of Chelex resin of 8% (w/v), 100 mmol/L of Surfactin, the solvent used to prepare reagent being purified water.
Control group 1 (sodium hydroxide solution): 1 mol/L of sodium hydroxide solution.
Control group 2 (dithiothreitol solution): 0.1 g of dithiothreitol, 0.78 g of sodium chloride, 0.02 g of potassium chloride, 0.112 g of sodium dihydrogen phosphate, 0.02 of potassium dihydrogen phosphate, purified water was added to a volume of 2 L.

Viscous biological sample: 1 tuberculosis-positive inactivated sputum sample that was viscous visually, was collected at SANWAY CLINICAL LABORATORIES INC., the sputum sample was fully mixed separately, and 3 aliquots (each 2 ml) for each sample were taken and marked as Nos. 1-3 for ready use.

### 17.2. Preparation of Nucleic Acid Samples to be Tested

Sample No. 1: the viscous biological sample liquefying agent of this disclosure in the experimental group was used as the liquefying agent. The liquefying agent at 4 times the volume of the sample was added to sample No. 1, mixed well, and stood still at room temperature for 10 min. The obtained liquid was used as the nucleic acid sample to be tested and for ready use. In this example, release of nucleic acids had been completed along with liquefaction, thus no additional operations for release and purification of nucleic acids were carried out.

Sample No. 2: the liquefying agent (sodium hydroxide solution) of control group 1 was used. Sodium hydroxide solution at 4 times the volume of sample was added to the sample No. 2, mixed well, and stood still at room temperature for 30 min until it was fully liquefied. Liquefaction was carried out while shaking and mixed evenly for 2-3 times. If the sputum was viscous, the time was needed to be extended until it was liquefied. After the obvious viscous substance, 1 mL of liquid was added into the centrifuge tube and centrifuged at 13000 rpm for 5 min. The supernatant was discarded and the precipitate was left. The precipitate was washed by addition of 1 mL of normal saline and centrifuged at 13000 rpm for 5 min. Supernatant was discarded. 1 mL of normal saline was added and mixed well to get a liquefied sample for ready use. Nucleic acid extraction reagent S1006 (Xiang machine registration No. 20150021) sold by SANSURE BIOTECH INC., was used to extract nucleic acid from the obtained liquefied sample to obtain the nucleic acid sample to be tested.

Sample No. 3: the liquefying agent (dithiothreitol solution) of control group 2 was used. Dithiothreitol solution at 4 times the volume of sample was added to the sample No. 3, mixed well, and stood still at room temperature for 30 min until it was fully liquefied. Liquefaction was carried out while shaking and mixed evenly for 2-3 times. If the sputum was viscous, the time was needed to be extended until it was liquefied, and a liquefied sample was obtained for ready use. Nucleic acid extraction kit S1006 (Xiang machine registration No. 20150021) sold by SANSURE BIOTECH INC., was used to extract nucleic acid from the obtained liquefied sample to obtain the nucleic acid sample to be tested.

Wherein, sample No. 2 and sample No. 3 adopted the traditional conventional preparation method of nucleic acid samples to be tested, which required nucleic acid extraction of liquefied samples.

Sample No. 2 and sample No. 3 were subjected to nucleic acid extraction according to the instructions of the commercial kit on sale. The steps were as follows:
(1) adding 600 μL of extraction solution 1 to each of appropriate amount of 1.5 mL centrifuge tubes according to the number of liquefied samples to be extracted.
(2) Adding 200 μL of liquefied sample to each tube; covering the tube cap, shaking and mixing well for 10 seconds, then heating at 95° C. for 10 minutes, and centrifuging instantaneously.
(3) Adding 100 μL of extraction solution 2 and 50 μL of magnetic bead suspension to each tube, shaking and mixing well for 10 seconds, and standing still at room temperature for 20 minutes.
(4) After instantaneous centrifugation, placing the centrifuge tube on the magnetic separator and slowly aspirating the solution after 3 minutes.
(5) Adding 600 μL of extraction solution 3 and 200 μL of extract solution 4 to each tube, shaking and mixing well for 5 seconds, and then placing the centrifuge tube in the magnetic separator again after instantaneous centrifugation.
(6) As supernatant was divided into two layers after about 3 minutes, inserting a pipette into the bottom of the centrifuge tube, slowly aspirating the liquid from the bottom completely and discarding, and after standing still for 1 minute, completely aspirating the residual liquid at the bottom of the tube and discarding.
(7) Adding 30-50 μL of eluent to elute the magnetic beads on the wall of the centrifuge tube to the bottom of the tube, pipetting and mixing well for 3-4 times, placing the centrifuge tube on the magnetic separator again for 3 minutes after standing still at room temperature for 10 minutes, and then placing the eluted nucleic acids into a new 1.5 mL centrifuge tube to obtain the nucleic acid sample to be tested, which was ready for use.

### 17.3. Detection of Nucleic Acid Samples

Sample No. 1 (combined with iPonatic mobile molecular detection system): Mycobacterium tuberculosis nucleic acid assay kit (PCR-fluorescent probe assay) sold by SANSURE BIOTECH INC., (SFDA Certified No.: 20173400166) was used combined with the iPonatic mobile molecular detection system provided by SANSURE BIOTECH INC., to carry out the nucleic acid detection of nucleic acid samples to be tested of sample No. 1, respectively. The PCR amplification procedure is as shown in Table 35.

TABLE 35
PCR amplification procedure for nucleic
acid sample detection of sample No. 1

| | | |
|---|---|---|
| Temperature | Time | Number of cycles |

| | | | |
|---|---|---|---|
| 94° C. | 1 | min | 1 cycle |
| 94° C. | 1 | s | 45 cycles |

| | |
|---|---|
| 57° C. | 5 s |
| | (Fluorescence |
| | acquisition) |

Samples No. 2 and No. 3 (combined with Real time quantitative fluorescent PCR instrument): Mycobacterium tuberculosis nucleic acid assay kit (PCR-fluorescent probe assay) sold by SANSURE BIOTECH INC., (SFDA Certified No.: 20173400166) was used combined with the Real time quantitative fluorescent PCR instrument (Shanghai Hongshi Medical Technology Co., Ltd., SLAN96P) to carry out the nucleic acid detection of nucleic acid samples to be tested of sample No. 2 and sample No. 3, respectively. The PCR amplification procedure is as shown in Table 36.

TABLE 36
PCR amplification procedure for nucleic acid sample detection of samples No. 2 and No. 3

| | | | | | Number of |
|---|---|---|---|---|---|
| Steps | | Temperature | Time | | cycles |
| 1 | UNG Enzymatic reaction | 50° C. | 2 | minutes | 1 |
| 2 | Taq Enzyme activation | 94° C. | 5 | minutes | 1 |
| 3 | Denaturation | 94° C. | 15 | seconds | 45 |
| | Annealing, extension, and fluorescence acquisition | 57° C. | 30 | seconds* | |
| 4 | Instrument cooling (not carried out) | 25° C. | 10 | seconds | 1 |

Note:
*indicates fluorescence acquisition. Step 4 (instrument cooling) is indicated in the product manual as an optional operation, which is not carried out in this example.

### 17.4. Test Results of Nucleic Acid Samples

The test results of nucleic acid samples to be tested of samples Nos. 1, 2 and 3 are as shown in Table 37 and Table 38.

According to Table 37 and Table 38, Mycobacterium tuberculosis from sputum samples (only needs 36 min) can be quickly detected with the viscous biological sample liquefying agent of this disclosure, while it takes about 4 hours or even longer to complete the nucleic acid detection with traditional liquefying agent. Moreover, while the viscous biological sample liquefying agent of this disclosure significantly shortens the detection time (compared with the control groups 1 and 2, the detection efficiency is increased by 5.8 times and 5.5 times, respectively), the Ct value of the PCR amplification curve is substantially the same as or even better than that of the control group 1 and the control group 2.

TABLE 37
Comparison of time-consuming from sample liquefaction to nucleic acid detection
when nucleic acid detection is performed with different liquefying agents

| | Liquefaction | Nucleic acid | Amplification | Total time- |
|---|---|---|---|---|
| Processing method | time | extraction time | time | consuming |
| Viscous biological sample liquefying | 10 min | | 26 min | About |
| agent of this disclosure | | | | 36 min |
| Control group 1 | more than | 120 min | 80 min | more than |
| (Sodium hydroxide method) | 45 min | | | 245 min |
| Control group 2 | more than | 120 min | 80 min | More than |
| (Dithiothreitol method) | 30 min | | | 235 min |

TABLE 38
PCR amplification test results

| Liquefying agent used | Ct value |
|---|---|
| Viscous biological sample liquefying agent of this disclosure | 28.01 |
| Control group 1 (sodium hydroxide solution) | 28.19 |
| Control group 2 (Dithiothreitol solution) | 28.05 |

17.5. The inventor further tried to combine the nucleic acid samples No. 2 and No. 3 to be tested with the iPonatic mobile molecular detection system for nucleic acid detection (the operation method can refer to the detection method for sample No. 1 in part 4.4). The results show that the Ct value of the PCR amplification curve is substantially consistent with the results of nucleic acid samples No. 2 and 3 in 4.4; and the whole detection time is still long, about 3.5 hours. It can be seen that after the viscous biological sample liquefying agent of this disclosure is used to realize direct amplification, the whole detection time can be shortened to 36 min, and the detection efficiency can be increased by more than 4.5 times.

### Example 18

4 sputum samples (I, II, III, and IV) that were viscous visually, were collected, each of these sputum samples was fully mixed separately, and 4 aliquots (2 ml of each) for each sample were taken and marked as Nos. 1-8 for ready use. Liquefaction was carried out according to the following methods for sample No. 1-8, respectively.

Experimental group (the technical solution in example 1 of this disclosure) was prepared with guaifenesin, sodium hydroxide, and zirconia beads. The final concentration was 100 mM of guaifenesin, 10 mM of sodium hydroxide, 1 g/ml of zirconia beads (the diameter was 1 mm), and the solvent used to prepare the reagent was purified water, which was used as the experimental group.
6 mL of reagent of the experimental group was added to sample No. 1;
6 mL of sodium hydroxide solution (1 M, 1000 mM) was added to sample No. 2;
6 mL of sodium hydroxide solution (0.5 M, 500 mM) was added to sample No. 3;
6 mL of sodium hydroxide solution (0.01 M, 10 mM) was added to sample No. 4;
6 mL of guaifenesin with a final concentration of 100 mM and sodium hydroxide aqueous solution with a final concentration of 10 mM were added to sample No. 5;
6 mL of guaifenesin with a final concentration of 100 mM and sodium hydroxide aqueous solution with a final concentration of 300 mM were added to sample No. 6;
6 mL of guaifenesin with a final concentration of 100 mM and sodium hydroxide aqueous solution with a final concentration of 400 mM were added to sample No. 7;
6 mL of guaifenesin with a final concentration of 100 mM and sodium hydroxide aqueous solution with a final concentration of 500 mM were added to sample No. 8;
The above samples were shaken and mixed well at room temperature for 5 minutes to observe the liquefaction effect;

Four liquefied samples obtained in the previous steps were aspirated with a pipette, the aspiration process was observed and recorded. The results are shown in Table 39.

TABLE 39

| Sample No | I | II | III | IV |
|---|---|---|---|---|
| No. 1 | The 4 samples were completely liquefied, and aspiratable and operatable by a 10 μL pipette | | | |
| No. 2 | Substantially liquefaction, unable to operate by a pipette tip | | | |
| No. 3 | Partial liquefaction, unable to operate by a pipette tip | | | |
| No. 4 | A little liquefaction, unable to operate by a pipette tip | | | |
| No. 5 | The 4 samples were completely liquefied, and aspiratable and operatable by a 10 μL pipette | | | |
| No. 6 | The 4 samples were completely liquefied, and aspiratable and operatable by a 10 μL pipette | | | |
| No. 7 | The 4 samples were completely liquefied, and aspiratable and operatable by a 10 μL pipette | | | |
| No. 8 | The 4 samples were completely liquefied, and aspiratable and operatable by a 10 μL pipette | | | |

The technical features of the above examples can be combined arbitrarily. In order to simplify the description, all possible combinations of the technical features in the above examples are not described. However, as long as there is no contradiction in the combination of these technical features, it should be considered as the scope of this specification.

The above-mentioned examples only illustrate several embodiments of this disclosure, and the description thereof is relatively specific and detailed, but it should not be understood as a limitation on the scope of protection of this disclosure. It should be noted that for those of ordinary skill in the art, without departing from the concept of this disclosure, a number of variations and improvements can be made, all of which belong to the scope of protection of this disclosure. In addition, it should be understood that after reading the above-mentioned teaching content, those skilled in the art can make various changes or modifications to this disclosure, and the obtained equivalent forms also fall within the scope of protection of this disclosure. It should also be understood that the technical solutions obtained by those skilled in the art through logical analysis, reasoning or limited numbers of tests on the basis of the technical solutions provided in this disclosure are all within the scope of protection of the claims attached to this disclosure. Therefore, the scope of protection of this disclosure should be subject to the appended claims, and the description and drawings can be used to explain the contents of the claims.

What is claimed is:

1. A viscous biological sample liquefying composition, comprising guaifenesin and a first strong base.

2. The viscous biological sample liquefying composition according to claim 1, wherein the first strong base is at least one of sodium hydroxide, potassium hydroxide, lithium hydroxide, calcium hydroxide, barium hydroxide or choline.

3. The viscous biological sample liquefying composition according to claim 1, further comprising rigid microparticles;
wherein, the rigid microparticles are made of a material including at least one of zirconia, silicon nitride, ceramsite, hard stainless steel, hard tungsten carbide, sintered corundum or agate.

4. A liquefying agent, comprising the viscous biological sample liquefying composition according to claim 1;
wherein the guaifenesin is of a concentration of 1 mmol/L to 1 mol/L and the first strong base is of a concentration less than 1 mol/L.

5. The liquefying agent according to claim 4, wherein the guaifenesin in the liquefying agent is of a concentration of 20 mmol/L to 1 mol/L;
and/or, the first strong base is of a concentration of 0.1 mmol/L to 500 mmol/L;
and/or, the liquefying agent is of a pH value equal to or greater than 10.

6. A viscous biological sample processing combination product comprising a liquefaction component, and further comprising a preservation component, a nucleic acid releasing component or a combination thereof; wherein the liquefaction component comprises the viscous biological sample liquefying composition according to claim 1;
the preservation agent is a mixture consisted of the preservation component; and the releasing agent is a mixture consisted of the nucleic acid releasing component.

7. The viscous biological sample processing combination product according to claim 6, wherein the preservation component comprises:
a1) a buffer component, for adjusting a preservation system to be a pH value of 6 to 8;
b1) an osmotic pressure regulating component; and
c1) at least one of trehalose, mannitol or glycerol.

8. The viscous biological sample processing combination product according to claim 7, wherein the preservation component satisfies one or more of the following characteristics:
(tb1) the buffer is for adjusting the preservation system to be a pH value of 6.8 to 7.6;
(tb2) the buffer component comprises citric acid;
(tb3) the osmotic pressure regulating component comprises betaine, inorganic cation or a combination thereof;
(tb4) the preservation component comprises trehalose, mannitol and glycerol;
(tb5) the preservation component further comprises one or more amino acids; and
(tb6) the preservation component further comprises urea.

9. The viscous biological sample processing combination product according to claim 8, wherein the preservation component satisfies one or more of the following characteristics:
(tb2a) the buffer component comprises 1 mmol/L to 5 mmol/L of citric acid;
(tb3a) the osmotic pressure regulating component comprises 0.1% to 1.2% (m/v) of sodium chloride and 0.1% to 1.2% (m/v) of potassium chloride;
(tb5a) the preservation component further comprises one or more amino acids and the amino acids in the preservation component are of a total concentration of 1 mol/L to 3 mol/L;
(tb4a) the trehalose in the preservation component is of a concentration of 0.5 mol/L to 1 mol/L;
(tb4b) the mannitol in the preservation component is of a concentration of 1.5% to 4.5% (m/v);
(tb4c) the glycerol in the preservation component is of a concentration of 2% to 10% (v/v); and
(tb6a) the preservation component further comprises 1% to 3% (m/v) of urea.

10. The viscous biological sample processing combination product according to claim 7, wherein the nucleic acid releasing component is a composition of component i), a composition of component ii), or a composition of component i) and component ii);
Component i) comprises: 0.1% to 2% (v/v) of Tween 20, 0.1% to 3% (v/v) of Triton X-100, 0.1% to 3% (v/v) of ethyl phenyl polyethylene glycol, 50 mmol/L to 1.25 mol/L of a second strong base, a first adsorbing agent and a second aqueous solvent;
Component ii) comprises: 0.01 mmol/L to 0.5 mmol/L of surfactant, 0.01% to 2% (w/v) of dodecylbenzene sulfonate, 0.05% to 1% (v/v) of ethanol and 100 mmol/L to 1.25 mol/L of a third strong base, with or without a second adsorbing agent.

11. The viscous biological sample processing combination product according to claim 10, wherein the nucleic acid releasing component satisfies one or more of the following characteristics:
(tr1) the first adsorbing agent comprises a chelating resin, trehalose or a combination thereof; the first adsorbing agent comprises Chelex resin, trehalose or a combination thereof;
and/or, the Chelex resin in the component i) is of a concentration of 1% to 15% (w/v);
(tr2) the first adsorbing agent comprises trehalose; and/or, the trehalose in the component i) is of a concentration of 0.5 mol/L to 1 mol/L;
(tr3) the second adsorbing agent is Chelex resin;
(tr4) the second strong base in the component i) is at least one of sodium hydroxide, potassium hydroxide, lithium hydroxide, calcium hydroxide, barium hydroxide or choline;
(tr5) the third strong base in the component ii) is at least one of sodium hydroxide, potassium hydroxide, lithium hydroxide, calcium hydroxide, barium hydroxide or choline;
(tr6) the second aqueous solvent in the component i) is 0.5 mmol/L to 500 mmol/L of Tris-HCl; and
(tr7) the surfactant in the component ii) is one or more of Surfactin, sodium lauryl sulfate or ethylenediamine tetraacetic acid.

12. A viscous biological sample liquefying agent, comprising a liquefaction component, a Surfactin and a fourth aqueous solvent;
wherein the liquefaction component comprises the viscous biological sample liquefying composition according to claim 1;
the viscous biological sample liquefying agent is of a pH value equal to or greater than 10.

13. The viscous biological sample liquefying agent according to claim 12, comprising 20 mmol/L to 1 mol/L of guaifenesin, the first strong base, 20 mmol/L to 200 mmol/L of Surfactin and the fourth aqueous solvent;
the viscous biological sample liquefying agent is compatible with an amplification system.

14. The viscous biological sample liquefying agent according to claim 12, wherein the viscous biological sample liquefying agent further comprises one or both of an adsorbing agent and rigid microparticles; the adsorbing agent is a chelating resin;
the rigid microparticles are made of a material including at least one of zirconia, silicon nitride, ceramsite, hard stainless steel, hard tungsten carbide, sintered corundum or agate.

15. A combination product for liquefying and preserving a viscous biological sample, comprising a liquefaction component and a preservation component;
wherein the liquefaction component comprises each component in the viscous biological sample liquefying agent according to claim 12;
the preservation component comprises:
a1) a buffer component, for adjusting a preservation system to be a pH value of 6 to 8;
b1) an osmotic pressure regulating component; and
c1) at least one of trehalose, mannitol or glycerol.

16. A kit comprising at least one of the viscous biological sample liquefying composition according to any one of claim 1.

17. The kit according to claim 16, wherein the kit satisfies one or more of the following characteristics:
(tk1) the kit comprises the viscous biological sample liquefying composition, and further comprises any one or more of a preservation agent, a releasing agent, an extraction agent, an amplification agent and a detection agent which are suitable for nucleic acids;
(tk2) the kit comprises a combination product for liquefying and preserving a viscous biological sample, and the kit further comprises any one or more of a releasing agent, an extraction agent, an amplification agent and a detection agent which are suitable for nucleic acids;
(tk3) the kit comprises a combination product for liquefying and releasing viscous biological sample, the kit further comprises one or more of a preservation agent, an extraction agent, an amplification agent and a detection agent which are suitable for nucleic acids;
(tk4) the kit comprises the viscous biological sample liquefying agent, and the kit further comprises one or more of a preservation agent, an amplification agent and a detection agent which are suitable for nucleic acids; and
(tk5) the kit comprises the combination product for liquefying and preserving a viscous biological sample, and the kit further comprises one or more of an amplification agent and a detection agent which are suitable for nucleic acids.

18. A method for liquefying a viscous biological sample, comprising steps of: mixing the viscous biological sample with a liquefaction component to obtain a first mixture, and incubating the first mixture;
wherein:
when the liquefaction component is the viscous biological sample liquefying composition according to claim 1, a second mixture is obtained after incubation;
the conditions for incubation satisfy one or more of the following characteristics:
(tc1) a pH value is equal to or greater than 10;
(tc2) guaifenesin in the first mixture is of a concentration of 1 mmol/L to 1 mol/L;
(tc3) the mixing is performed by stirring or shaking;
(tc4) a temperature for incubation is selected from 18 to 35° C.;
(tc5) a time for incubation is less than 30 mins.

19. A method for liquefying a viscous biological sample, comprising steps of: mixing the viscous biological sample with a liquefaction component to obtain a first mixture, and incubating the first mixture;
when the liquefaction component is selected from the viscous biological sample liquefying agent according to claim 12, a liquefied product is obtained after incubation;
the conditions for incubation satisfy one or more of the following characteristics:
(tc1) a pH value is equal to or greater than 10;
(tc2) guaifenesin in the first mixture is of a concentration of 1 mmol/L to 1 mol/L;
(tc3) the mixing is performed by stirring or shaking;
(tc4) a temperature for incubation is selected from 18 to 35° C.;
(tc5) a time for incubation is less than 30 mins.

20. The method for liquefying a viscous biological sample according to claim 19, wherein the viscous biological sample is sputum, cervical mucus, nasopharyngeal swab or oral swab.
